(19) United States
(12) Patent Application Publication
(10) Pub. No.: US 2016/0339073 A1
(43) Pub. Date: Nov. 24, 2016

(54) RNA AND PROTEIN NETWORKS THAT LOCALLY CONTROL BRAIN WIRING DURING DEVELOPMENT

(71) Applicant: President and Fellows of Harvard College, Cambridge, MA (US)

(72) Inventors: Jeffrey Macklis, Brookline, MA (US); Alexandros Poulopoulos, Cambridge, MA (US); Alexander James Murphy, Boston, MA (US)

(21) Appl. No.: 15/144,660

(22) Filed: May 2, 2016

Related U.S. Application Data
(60) Provisional application No. 62/155,436, filed on Apr. 30, 2015.

(51) Int. Cl. A61K 38/02; A61K 31/7088

(52) U.S. Cl. CPC A61K 38/02; A61K 31/7088

(57) ABSTRACT

Disclosed herein are methods of purifying growth cones from specific projections in the brain comprising a combination of in vivo labeling, subcellular fractionation, and fluorescent small particle sorting. The methods disclosed herein enable the quantitative profiling of the proteomes and transcriptomes of growth cones and their parent cell bodies from callosal projection neurons. Also disclosed herein are specific RNA and protein networks involved in callosal circuit formation and core growth cone proteomic machinery. The inventions disclosed herein are adaptable to any projection in the brain, providing insight into the molecular networks that control the wiring of specific neural circuits in vivo.

### RELATED APPLICATION(S)

This application claims the benefit of U.S. Provisional Application No. 62/155,436, filed Apr. 30, 2015, the entire teachings of which are incorporated herein by reference.

### GOVERNMENT SUPPORT

This invention was made with government support under NS049553, NS075672, NS045523 and NS041590 awarded by the National Institutes of Health. The government has certain rights in the invention.

### BACKGROUND OF THE INVENTION

One of the fundamental goals of neuroscience is to understand how neural circuits in the brain are structurally and functionally connected through the actions of molecules, genes, and neurons. With the development of several technologies for molecular profiling to unravel the complexity of cell types in the brain, immense progress has been made in elucidating distinct classes of neurons (Arlotta et al., 2005; Doyle et al., 2008; Heiman et al., 2008; Pollen et al., 2014; Zeisel et al., 2015) and in characterizing transcriptional mechanisms that specify their identity (Jessell, 2000; Molyneaux et al., 2007). However, current molecular profiling approaches have not provided much insight into how circuits are wired during development, especially since they neglect the molecular networks downstream of genetic programs that actually implement circuit wiring.

The technical development of optogenetics (Boyden et al., 2005) and recent advances made in high-throughput imaging using Ca²⁺ indicators (Grienberger and Konnerth, 2012) and viral tracers (Oh et al., 2014) have enabled immense progress toward mapping the connectivity of neural circuits. Several large-scale circuit mapping initiatives have been established to apply these technologies in order to understand how neural circuit organization can influence the onset, expression, and course of brain disorders (Insel et al., 2013). However, while these approaches are informative for understanding mature neural circuit organization and function, elucidating the molecular networks and mechanisms that wire neural circuits is likely required to identify candidate drug targets for therapeutic intervention to treat certain brain disorders, particularly neurodevelopmental disorders such as the autism spectrum disorders.

### SUMMARY OF THE INVENTION

Disclosed herein are compositions and methods for profiling growth cones in vivo from distinct neuron subtypes; these compositions and methods contribute substantially to the understanding of how specific neural circuits are wired during development. Also disclosed are novel compositions, methods and candidate drug targets for therapeutic intervention and follow up which are useful for diagnosing, treating and monitoring the development of neurologic conditions (e.g., neurodegenerative or neuropsychiatric conditions). In some embodiments, the inventions disclosed herein relate to methods of purifying projection-specific growth cones, such methods comprising (a) performing in vivo labeling of a desired neuron population; (b) performing subcellular fractionation of the labeled neuron population; (c) performing fluorescent small particle sorting of the fractionated labeled neuron population; and (d) isolating the projection-specific growth cones. In some embodiments, the subcellular fractionation comprises isopycnic subcellular fractionation.

Also disclosed herein are methods of treating a subject with a neurologic condition, the method comprising administering to the subject a composition comprising an effective amount of an agent that modulates expression of at least one nucleic acid enriched in neuron growth cones relative to neuron cell bodies, thereby treating the neurologic condition.

In certain embodiments, disclosed herein are methods of assessing a neurologic condition in a subject, such methods comprising a step of identifying differential expression of one or more nucleic acids enriched, in neuron growth cones of the subject relative to the expression of the same one or more nucleic acids in neuron growth cones of a normal subject, wherein if the one or more nucleic acids are differentially expressed, the subject has or is at risk of developing the neurologic condition.

In certain embodiments, the inventions disclosed here relate to methods of monitoring progression or amelioration of a neurologic condition in a subject, such methods comprising a step of identifying differential expression of one or more nucleic acids in neuron growth cones of the subject relative to the expression of the same one or more nucleic acids in neuron growth cones of a normal subject, wherein if the one or more nucleic acids are differentially expressed, the subject has or is at risk of developing the neurologic condition, and monitoring the expression of the one or more nucleic acids over time, wherein alteration of the expression of the one or more nucleic acids over time is indicative of progression or amelioration of the condition.

In certain aspects, the inventions are directed to methods of regenerating damaged neuronal circuitry in a subject, such methods comprising contacting neuron growth cones of the subject with an effective amount of an agent that selectively targets at least one marker selectively expressed by neuron growth cones relative to neuron cell bodies, causing the neuron growth cones to project toward their targets to form synapses and thereby restore the damaged neuronal circuitry.

In some embodiments, also disclosed herein are methods of identifying a candidate agent for the treatment of a neurologic condition, the methods comprising identifying agents that preferentially modulate the expression of one or more nucleic acids enriched in neuron growth cones compared to neuron cell bodies, wherein those agents that preferentially modulate the expression of the one or more nucleic acids enriched in the neuron growth cones are candidate agents for the treatment of the neurologic condition.

In some aspects, the inventions disclosed herein relate to methods of directing projection of a neuron axon in vivo, such methods comprising a step of contacting growth cones of the neuron axon with one or more canonical cues and thereby directing projection of the neuron axon.

In certain embodiments, the nucleic acid comprises RNA. In certain embodiments, the nucleic acid comprises noncoding RNA. In certain embodiments, the nucleic acid comprises mRNA. In still other embodiments, the nucleic acid is selected from the group consisting of α-Tub, β-Tub, γ Actin, Basp1, Crmp2, MAP1B, Ncam, Dynein, Basp1, L1cam, Contactin, Gprin1, Stxbp1, Dync1h1, Syn1, Cxadr, Gpm6a, Stx1b, Psmd1 and Mapre1. In some embodiments, modulating expression comprises increasing expression of at least one nucleic acid. In other embodiments, modulating expression comprises decreasing expression of at least one nucleic acid.

In some embodiments, the nucleic acid is more than about 2-fold enriched in the neuron growth cones relative to the neuron cell bodies. In some embodiments, the nucleic acid is more than about 10-fold enriched in the neuron growth cones relative to the neuron cell bodies. In still other embodiments, the nucleic acid is more than about 20-fold enriched in the neuron growth cones relative to the neuron cell bodies.

In some embodiments, the agent is selected from the group consisting of small organic molecules, oligosaccharides, polysaccharides, peptides, proteins, peptide analogs, miRNA, siRNA, antisense RNA, and any combination thereof.

In some embodiments, the marker comprises a protein. In certain aspects, the marker comprises a receptor. In some embodiments, the marker comprises GAP-43.

In some embodiments of the foregoing methods, such methods further comprise a step of contacting the neuron growth cone with one or more canonical cues. For example, one or more canonical cues selected from the group consisting of Netrins, Slits, Semaphorins and Ephrins.

Also disclosed herein are methods of forming or restoring a neuronal circuit, such methods comprising a step of contacting one or more of neuron growth cones with an effective amount of an agent that selectively targets the neuron growth cones relative to neuron cell bodies, causing the neuron growth cones to project towards their targets to form a synapse and thereby forming or restoring the neuronal circuit. In certain aspects, such agent comprises one or more canonical cues (e.g., a canonical cue selected from the group consisting of Netrins, Slits, Semaphorins and Ephrins). In certain aspects, the agent is selected from the group consisting of small organic molecules, oligosaccharides, polysaccharides, peptides, proteins, peptide analogs, miRNA, siRNA, antisense RNA, and any combination thereof.

In certain aspects, the marker comprises a protein. In certain aspects, the marker comprises a receptor. In yet other aspects, the marker comprises Growth Associated Protein 43 (GAP-43). In certain other aspects, the marker comprises noncoding RNA. In still other embodiments, where the neuron comprises a cell body and growth cones, the marker (e.g., GAP-43) is enriched in the growth cones relative to the cell body. For example, in certain embodiments, the marker is more than about 2-fold, about 3-fold, about 4-fold, about 5-fold, about 6-fold, about 7-fold, about 8-fold, about 9-fold, about 10-fold or more enriched in the growth cones relative to the cell body.

The methods and assays disclosed herein are useful for diagnosing, monitoring, treating or otherwise ameliorating a neurologic condition, for example, a neurodegenerative, neuropsychiatric or a neurodevelopmental disorder. In certain aspects the neurodegenerative disorder is selected from the group consisting of Huntington's disease, dentatorubropallidoluysian atrophy, Kennedy's disease, spinocerebellar ataxia, fragile X syndrome, fragile XE mental retardation, Friedreich's ataxia, myotonic dystrophy, spinocerebellar ataxia type 8, spinocerebellar ataxia type 12, Alexander disease, Alper's disease, Alzheimer disease; amyotrophic lateral sclerosis, ataxia telangiectasia, Batten disease, Canavan disease, Cockayne syndrome, corticobasal degeneration, Creutzfeldt-Jakob disease, ischemia stroke, Krabbe disease, Lewy body dementia, multiple sclerosis, multiple system atrophy, Parkinson's disease, Pelizaeus-Merzbacher disease, Pick's disease, primary lateral sclerosis, Refsum's disease, Sandhoff disease, Schilder's disease, spinal cord injury; spinal muscular atrophy, Steele Richardson-Olszewski disease, and Tabes dorsalis. In certain aspects, the neurologic condition is selected from the group consisting of an autism spectrum disorder, schizophrenia, bipolar disorder, and Rett syndrome.

The above discussed, and many other features and attendant advantages of the present inventions will become better understood by reference to the following detailed description of the invention.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIGS. 1A-1D demonstrate that axons intrinsically know where to project in vivo. FIG. 1A illustrates an ectopic micro-transplantation strategy. Neurons from the lateral cortex of a GFP transgenic mouse were dissected and mixed in cell suspension with neurons derived from the medial cortex of a TdTomato transgenic mouse. Lateral-derived and medial-derived neurons were transplanted into the medial cortex of littermate dark hosts. Medial-derived neurons serve as an internal control. FIG. 1B illustrates in vitro culture of lateral-derived GFP neurons and medial-derived TdTomato neurons (DIV 7). FIG. 1C shows coronal section 7 days post transplantation. FIG. 1D shows quantification (n=6) of projections of medial- or lateral-derived neurons transplanted into medial cortex.

FIGS. 2A-2D illustrate that subcellular fractionation yields enriched population of membrane-bound growth cones. FIG. 2A presents the subcellular fractionation protocol. Growth cones are isolated from interface 1. PNH, postnuclear homogenate; 0.32, 320 mM sucrose; 0.83, 830 mM sucrose. FIG. 2B (left) shows Ponceau S staining showing equal amount of protein loading from Interface 1 and PNH, and (right) Western blot of growth cone (GAP-43) and dendritic (MAP-2) markers. FIG. 2C shows Western blot characterization of distinct fractions probed with indicated subcellular markers. FIG. 2D illustrates the growth cone trypsin protection assay, showing that (left) combinations of detergent and trypsin were applied to Interface I, enabling discrimination between membrane-protected proteins and cytosolic proteins, and that (right) Western blot indicates that Interface 1 contains growth cones with intact membranes.

FIGS. 3A-3E illustrate that growth cones contain multiple and heterogeneous RNA species. FIG. 3A shows Bioanalyzer distributions of RNA purified from indicated fractions. FIG. 3B shows growth cone RNase protection assay and illustrates that (left) combinations of detergent and RNase were applied to isolated growth cones, enabling discrimination between membrane-protected RNA and cytosolic RNA, and that (right) Bioanalyzer analysis indicates that RNA in growth cone fraction is from membrane-enclosed growth cones, not from soluble RNA/protein complexes. FIG. 3C shows native RNA gel of purified RNA from PNH and Interface 1. FIG. 3D shows Bioanalyzer distribution of growth cone mRNA after poly(A+) selection with oligo (dT)₂₅ beads. FIG. 3E shows RT-PCR with primers for indicated transcripts.

FIGS. 4A-4D shows purification of fluorescently labeled growth cones using fluorescent small particle sorting. FIG. 4A presents size distribution of growth cones as measured by Nanoparticle tracking analysis. FIG. 4B represents a forward-side scatter profile of growth cone particles and a mixture of size standard beads. 0.3 μm, 0.5 μm, 0.8 μm, and 1.0 μm sized fluorescent beads were used for fluorescence gating and estimation of growth cone size, showing (left) beads alone, (center) growth cones, and (right) overlap of beads and growth cones. FIG. 4C shows growth cones from GFP+ or TdTomato+ transgenic mice were mixed for fluorescent small particle sorting. FIG. 4D shows that (left) GFP+ and TdTomato+ growth cones were easily distinguished and (right) after gating and collection, GFP+ growth cones were subjected to a re-sort, demonstrating successful purification of GFP-gated growth cones.

FIGS. 5A-5E illustrate purification of circuit-specific growth cone populations. FIG. 5A shows coronal section at P4. In utero electroporation was performed at E15.5 to express CAG>myr-TdTomato into CPN. FIG. 5B illustrates the strategy to isolate TdTomato+CPN growth cones. FIG. 5C represents a forward scatter-fluorescence profile of TdTomato+ growth cones compared to size standard beads. FIG. 5D shows coronal section at P7. Corticothalamic projection neurons (CThPN) were genetically labeled by crossing NTSR1::Cre and Rosa26^{flax-STOP-TdTomato} mice. FIG. 5E represents a forward scatter-fluorescence profile of TdTomato+CThPN growth cone.

FIGS. 6A-6G illustrate that comparative proteomics reveals CPN-specific proteome. FIG. 6A shows that increasing numbers of sorted TdTomato+CPN growth cones as described in FIGS. 5C-5D were analyzed by proteomics. Approximately 1.5 million growth cones are required for full coverage and linearity over the quantifiable dynamic range. FIG. 6B shows the complete projection-specific growth cone interaction network constructed from purified TdTomato+CPN growth cones. The highest enriched protein networks are indicated (receptor/cytoskeleton signaling, ribosome and translation machinery, and proteasome). FIG. 6C shows that whole mount view of brain at P2. Dual in utero electroporation was performed to label medial CPN with GFP and lateral CPN with TdTomato. FIG. 6D shows coronal section at P2 showing medial, GFP+CPN axons and lateral, TdTomato+CPN axons. FIG. 6E shows fluorescence profile of medial, GFP+CPN axons and lateral, TdTomato+CPN axons. Growth cone populations were purified based on indicated fluorescence gates. FIG. 6F shows the distribution of proteins in indicated samples detected by proteomics. Note that the highest abundance proteins are consistent across samples. FIG. 6G shows the distribution of proteins in medial and lateral growth cones. Plotted is the ratio of protein abundance as measured by proteomics in medial versus lateral growth cones. Annotated are proteins that are lateral-specific or medial-specific.

FIGS. 7A-7F represent comparative subcellular profiling enabled by parallel purification of cell bodies and growth cones. FIG. 7A illustrates a strategy to dually purify cell bodies and growth cones using in utero electroporation and anatomical dissection. A CAG>H2B-EGFP-2A-myr-TdTomato construct is expressed in CPN, labeling cell bodies with GFP and axons with TdTomato. At P4, the two hemispheres are physically separated and processed independently: cell bodies from the electroporated hemisphere are dissociated into single cells and purified using FACS, whereas growth cones are isolated from the contralateral hemisphere and purified using fluorescent small particle sorting. FIG. 7B shows coronal section at P4. FIG. 7C (left) shows GFP+ cell bodies purified using FACS and (right) Bioanalyzer distribution of RNA purified from sorted cell bodies. FIG. 7D (left) shows TdTomato+ growth cones purified using fluorescent small particle sorting, and (right) Bioanalyzer distribution of RNA purified from sorted growth cones. FIG. 7E shows RNA-seq reads of libraries prepared from growth cone or cell body RNA. Note that cell body-restricted Xist transcripts are absent from growth cone RNA-seq libraries, while the known growth cone-localized β-actin transcript is expressed. FIG. 7F shows the distribution of transcripts in growth cones and cell bodies. Plotted is (FPKM in growth cones)/(FPKM in cell bodies).

FIGS. 8A-8B shows subcellular fractionation yields enriched population of growth cones devoid of other subcellular compartments. FIG. 8A shows a subcellular fractionation protocol. Growth cones are isolated from Interface 1. PNH, postnuclear homogenate; 0.32, 320 mM sucrose; 0.83, 830 mM sucrose. FIG. 8B shows a Western blot characterization of distinct fractions probed with indicated subcellular markers.

FIGS. 9A-9B demonstrate that RpLIOa is not expressed in neocortical growth cones. FIG. 9A is a coronal section at P4 from Emx::Cre; Rosa26^{flax-STOP-eGFP-RpL10a} mice. FIG. 9B shows a Western blot analysis of RpL10a, eGFP, and GAP-43 expression in indicated wild-type or Cre+Rosa26^{flax-STOP-eGFP-RpL10a} mice. Note that eGFP-RpL10a expression is restricted to Cre+ neurons, but neither endogenous RpL10a nor the eGFP-RpL10a fusion protein are expressed in neocortical growth cones.

FIGS. 10A-10C show that growth cones have distinct ribosome proteins. FIG. 10A shows a Western blot characterization of distinct fractions probed with growth cone marker (GAP-43) or ribosome proteins (FIG. 10B) growth cone trypsin protection assay. Combinations of detergent and trypsin were applied to the growth cone fraction, enabling discrimination between membrane-protected ribosome proteins and cytosolic ribosome proteins. FIG. 10C shows a Western blot analysis of the growth cone trypsin protection assay. Additional fractions are included for abundance comparisons.

FIGS. 11A-11C illustrate parallel purification of CPN cell bodies and growth cones. FIG. 11A (top) depicts a strategy to dually purify cell bodies and growth cones using in utero electroporation and anatomical dissection. A CAG>H2B-EGFP-2A-myr-TdTomato plasmid is expressed in CPN, labeling cell bodies with GFP and axons with TdTomato. At P4, the two hemispheres are physically separated and processed independently: cell bodies from the electroporated hemisphere are dissociated into single cells and purified using FACS, whereas growth cones are isolated from the contralateral hemisphere and purified using fluorescent small particle sorting, and (bottom) coronal section at P4. FIG. 11B shows forward scatter-fluorescence plot for GFP+ cell body purification using FACS. FIG. 11C shows forward scatter-fluorescence plot for TdTomato+ growth cone purification using fluorescent small particle sorting.

FIG. 12A-12B demonstrate that comparative subcellular profiling reveals distinct composition of ribosome proteins in growth cones. FIG. 12A shows the ribosome protein composition of the 40S subunit. For each 40S subunit protein, the expression in CPN cell bodies (green square), CPN growth cones (red square), and total cortex growth cones (black square) is shown. Also included are the expression results from two previously published growth cone proteomes, Nozumi et al., 2009 (grey square), and Estrada et al., 2012 (blue square). FIG. 12B shows the ribosome protein composition of the 60S subunit, as described in FIG. 12A.

FIGS. 13A-13D show Subcellular localization of epitope-tagged ribosome proteins. FIG. 12A shows HA-tagged RpS25 expression in NIH/3T3 cells. Immunocytochemistry (ICC) for Crimson-ER marker (blue), RpS25-HA (green), RpL28 (red). Center and right panels are single fluorescent channels for RpS25-HA and RpL28, respectively. FIG. 13B show ICC for RpS25-HA (green), RpS6 (red), and Crimson-ER marker (blue). FIG. 13C shows ICC for RpS17-HA (green), RpS6 (red), and Crimson-ER marker (blue) (D) ICC for RpL22-HA (green), RpS6 (red), and Crimson-ER marker (blue).

FIGS. 14A-14B demonstrate ribosome affinity purification from growth cones in vivo. FIG. 12A depicts a strategy to dually purify RpS25-HA tagged ribosomes from CPN cell bodies and growth cones in vivo using in utero electroporation and anatomical dissection. A CAG>RpS25-HA plasmid is expressed in CPN, labeling CPN ribosomes. At P4, the two hemispheres are physically separated and processed independently for ribosome affinity purification: the electroporated hemisphere is processed for CPN cell body ribosome affinity purification, whereas the contralateral hemisphere is processed for CPN growth cone ribosome affinity purification. FIG. 12B shows Bioanalyzer distributions: (Top) Beads-only control (Center) RNA purified from CPN cell body ribosomes (Bottom) RNA purified from CPN growth cone ribosomes.

FIG. 15 illustrates the multimodal labeling for purification of any projection-specific growth cone population.

FIG. 16 illustrates the purification of subtype-specific and area-specific growth cones from neurons in vivo via genetic, electroporation and/or physical labeling, followed by growth cone isolation, enrichment and fluorescent small particle sorting.

FIG. 17 shows the results of subcellular proteomics analyses revealing the substantial depth of the growth cone-enriched proteome.

FIG. 18 illustrates that the isolated growth cones purified in accordance with the methods disclosed herein have a size distribution of approximately 0.3-1 μM, which matched electron microscopy sizing.

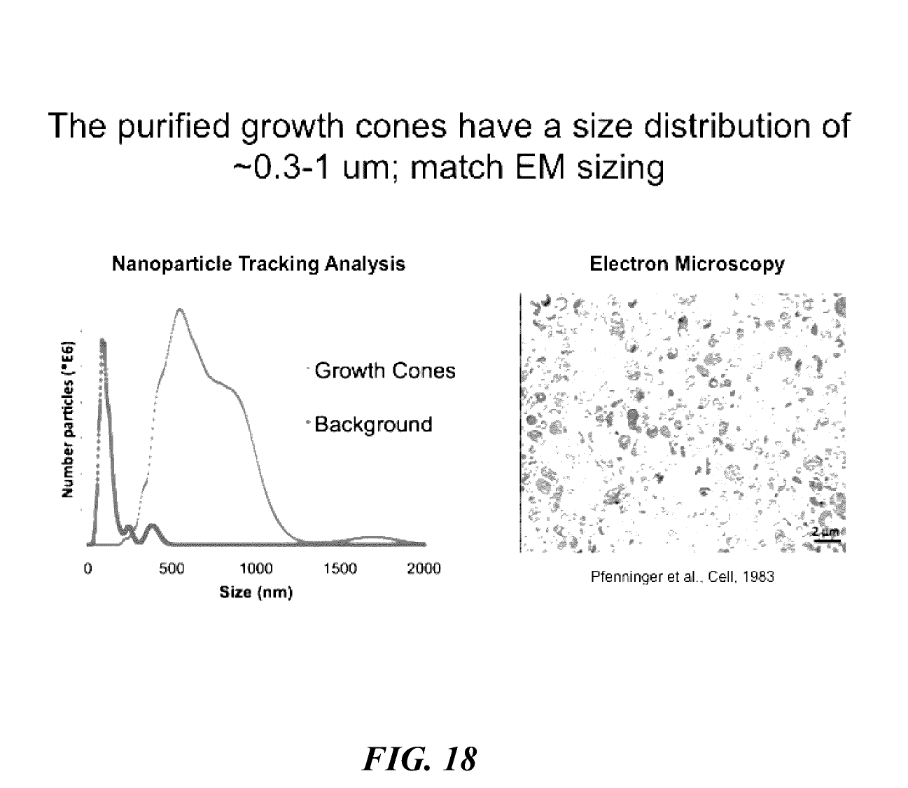

### DETAILED DESCRIPTION OF THE INVENTION

The molecular machineries of neuron growth cones control the formation of neural circuits in the developing brain and enable axons to navigate toward their targets over many days and far from their somas. Although great progress has been made in elucidating axon guidance cues and their growth cone receptors, we still lack an understanding of the projection-specific RNA and protein networks in growth cones that likely control the wiring of specific circuits in vivo. Disclosed herein are novel compositions, methods and assays useful for characterizing growth cone molecular networks and which will contribute substantially to the development of therapies to correct wiring abnormalities. After first demonstrating that axons have intrinsic mechanisms for wiring neural circuits, the present inventors describe methods and assays for purifying projection-specific growth cones by combining in vivo labeling of desired neuron populations, subcellular fractionation, and newly-developed fluorescent small particle sorting. As more particularly described in the Examples, the present inventors then apply such methods and assays to profile growth cones from callosal projection neurons (CPN). The present inventors then elucidate the full-depth transcriptomes and proteomes of CPN growth cones and their parent cell bodies isolated in vivo. Through comparative analysis, patterns of RNA subcellular localization are uncovered, with several previously unrecognized features that may control the wiring of specific brain circuits.

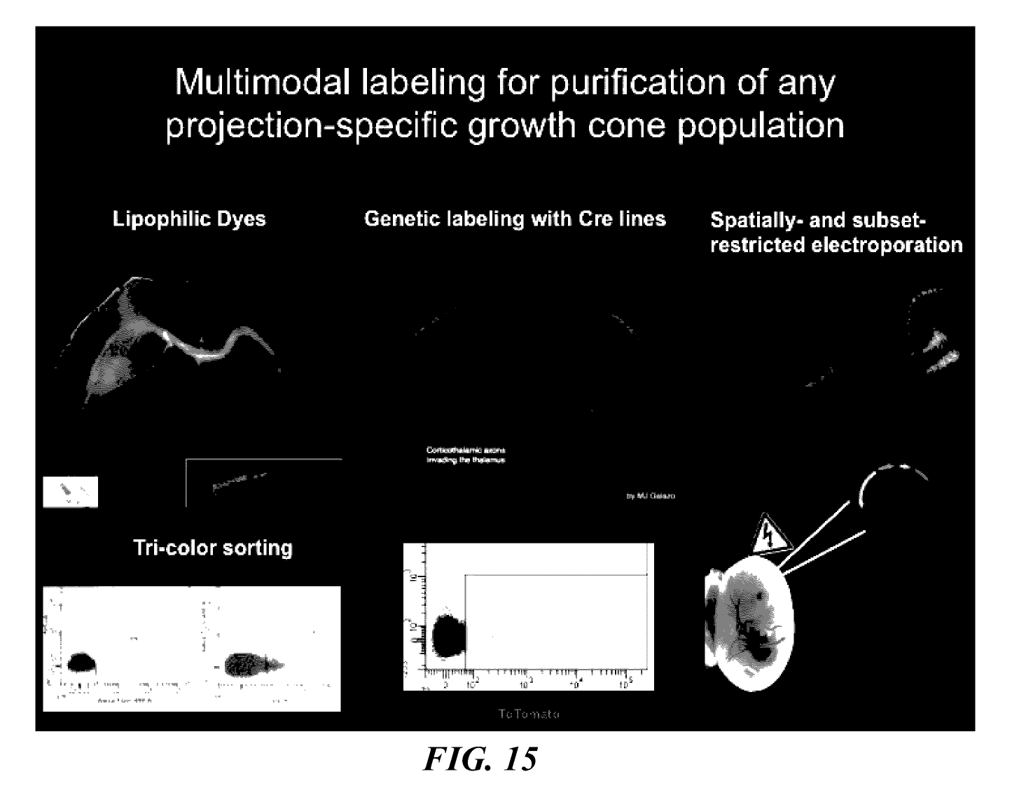

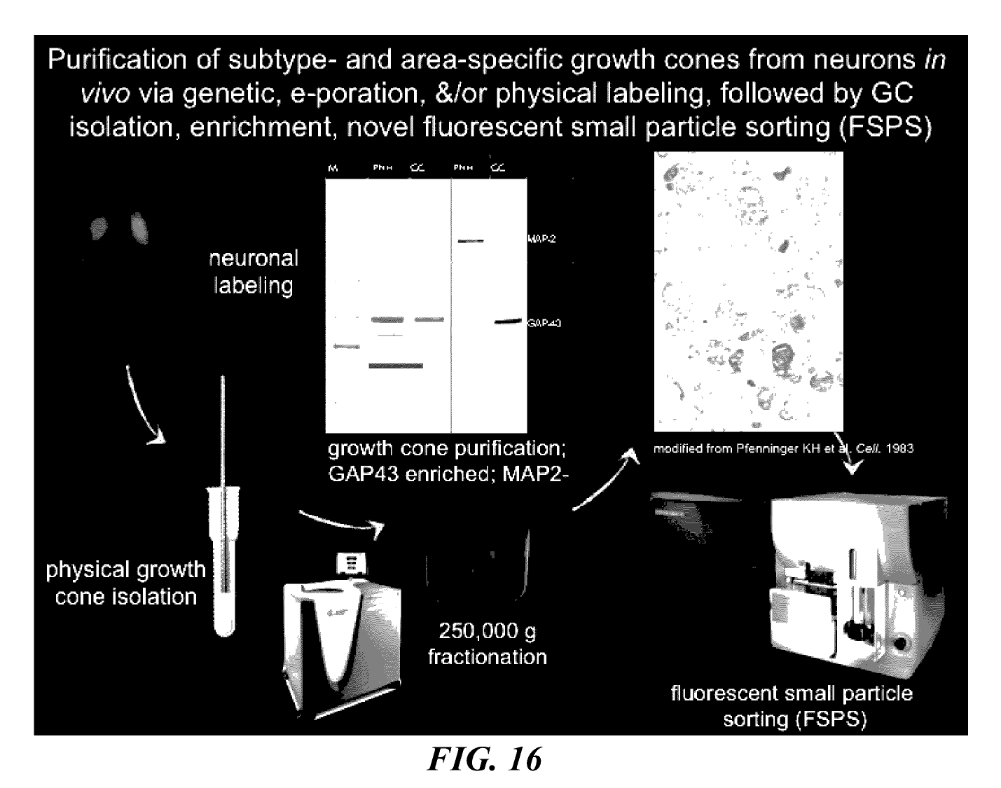

To understand how specific projection neurons make wiring decisions, the present inventors focused on CPN, which connect the two cerebral hemispheres through the corpus callosum. Disclosed herein are methods and compositions developed to profile and quantify the full-depth transcriptomes and proteomes of CPN growth cones and their parent cell bodies isolated in vivo. Also disclosed are methods of labeling any projection-specific growth cone population, isolating labeled growth cones from specific neuron subtypes and determining which isolated growth cones are amenable to full depth proteomic and RNAseq analysis (FIGS. 15 and 16).

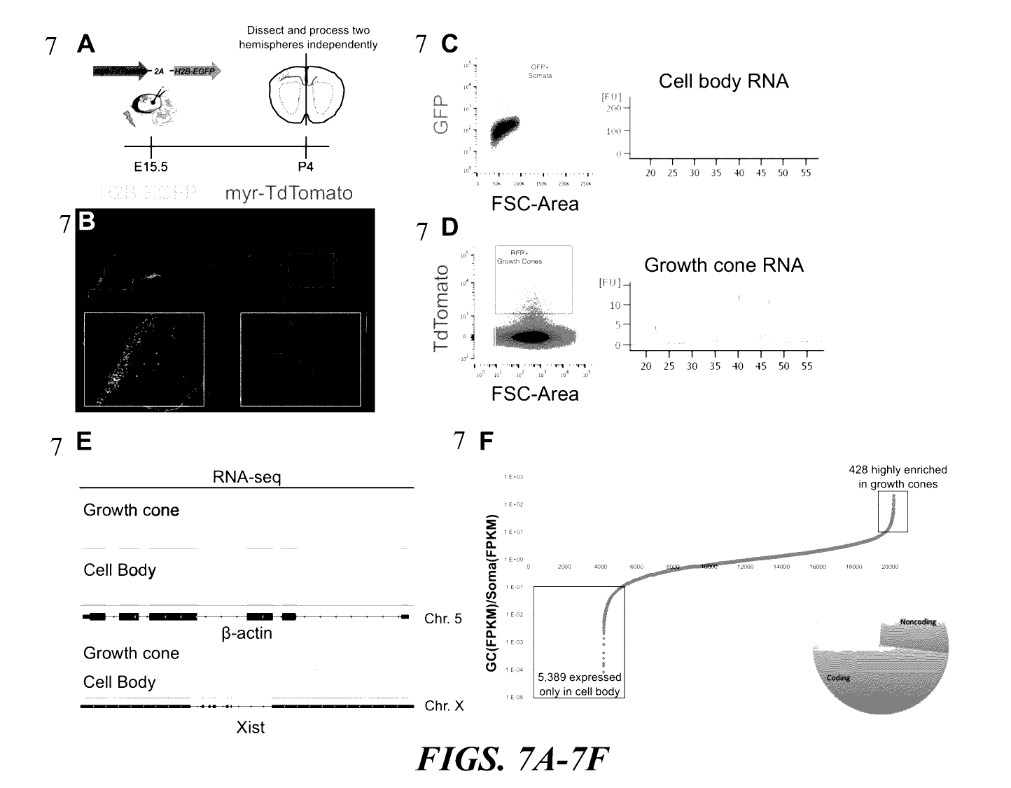

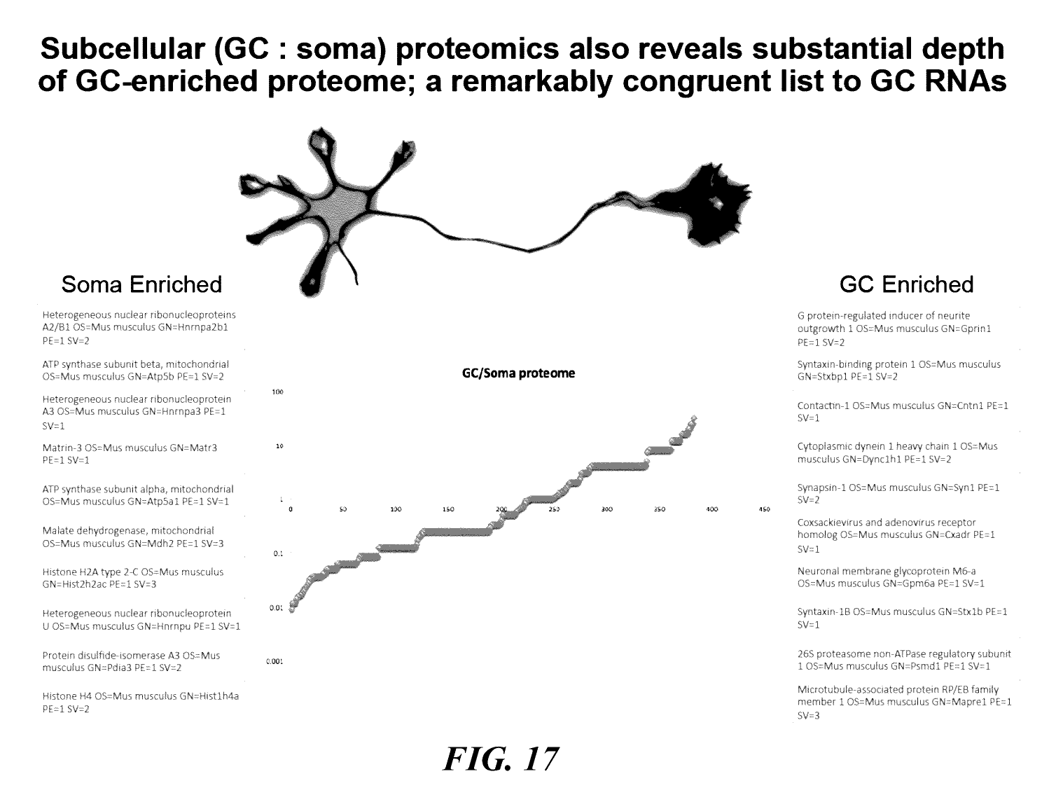

Using the comparative methods and assays disclosed herein, general patterns of RNA and protein subcellular localization were uncovered, with several previously unrecognized features, relating to control of the wiring of specific brain circuits. First, as illustrated in FIGS. 7A-7F, while most nucleic acid transcripts were expressed at similar levels in cell bodies and growth cones, a select subset are more than 10-fold enriched in growth cones compared to cell bodies, suggesting active localization of those transcripts to the growth cone. By then correlating transcriptomic and proteomic data, the spatial relationship between coding transcripts and their encoded proteins was characterized. Intriguingly, many of the growth cone-enriched nucleic acid transcripts were noncoding RNA with unknown function. After early “programming” by the nucleus, growth cones appear to be autonomous, mini-cellular units that may underlie development, disease and degeneration and their molecular networks are not observed as somal transcripts. As illustrated in FIG. 17, subcellular proteomics analyses revealed the substantial depth of the growth cone-enriched proteome. Further, disclosed herein is the finding that growth cones appear to have distinct ribosomes. These ribosomes lack several large subunit proteins, raising the intriguing possibility of growth cone-specific translational mechanisms for selective mRNA expression.

The methods, assays and compositions disclosed herein are readily adaptable to other projection types in the brain, enabling high-throughput, quantitative investigation of RNA and protein controls over circuit development and, potentially, the regeneration of damaged circuitry. In addition, the methods and assays disclosed herein are scalable to include epigenetic profiling, enabling full investigation of DNA, RNA, and protein networks that collectively coordinate brain wiring during development. The insights derived from the present inventions exemplify its capacity to quantify and characterize the molecular and translational mechanisms that control specific brain wiring at the subcellular level in vivo.

Since the completion of the Human Genome Project, several large-scale genome-wide analyses have demonstrated strong correlative associations of mutations across thousands of genomic loci with several classes of diseases, including mental illness and neurological disorders. Concurrently, several large interdisciplinary initiatives, including Brain Research through Advancing Innovative Neurotechnologies (BRAIN), the Allen Brain Institute, and the Human Brain Project, have started to apply high-throughput imaging and computational technologies toward deciphering the circuits that underlie normal brain function (Manji et al., 2014). By integrating these and other large-scale data sets, these initiatives likely will contribute to the understanding of how genetics and neural circuit organization can influence the onset, expression, and course of neurological disorders (Fornito et al., 2015).

The present inventors propose that novel insights into brain wiring result from the development of methods of profiling molecular networks in growth cones. Development of such methods enables the elucidation of the perturbed molecular networks in neurological disorders that cause wiring abnormalities. Using these insights, novel drug targets to treat neurodevelopmental disorders can be identified, and a robust biomarker system to validate the efficacy of drugs developed to target molecules implicated in wiring abnormalities may be obtained.

After the initial publication of the reference human genome in 2001 (Lander et al., 2001; Venter et al., 2001), many subsequent large-scale sequencing efforts have substantially advanced the identification of genes associated with disease. Starting in 2005 with a report investigating the genetic basis of age-related macular degeneration (Klein et al., 2005), genome-wide association studies (GWAS) have identified the presence of genomic regions with common single nucleotide polymorphisms (SNPs) that have strong correlations with particular disorders. Critical to the logic of utilizing GWAS analysis is the common-disease/common-variant hypothesis, which postulates that common diseases are driven by multiple common variants (Manolio et al., 2009). Thus, by analyzing SNPs across thousands of individuals, particular genomic locations linked to particular disorders may be identified for further analysis.

Across all diseases, the vast majority of GWAS-identified SNPs lie in intergenic or intronic regions (approximately 88%), and therefore are likely to impact gene regulation (Edwards et al., 2013). Interestingly, however, GWAS analyses of mental illness and neurological disorder patients have identified higher associations with protein-coding genes. For example, 75% of GWAS loci associated with schizophrenia are protein-coding genes (Ripke et al., 2014). In autism spectrum disorder (ASD) patients, many protein-coding and microRNA genes are implicated in ASD pathophysiology (Abu-Elneel et al., 2008; Hussman et al., 2011; Weiss et al., 2009). By bioinformatically integrating findings from several ASD GWAS reports, one group reported that axon outgrowth and synaptic function networks were highly enriched networks associated with ASD (Poelmans et al., 2013).

Axon outgrowth/guidance pathways also have been implicated in diseases not viewed as neurodevelopmental disorders. For example, by analyzing a large cohort of Parkinson's disease patients, one GWAS reported that SNPs in the axon guidance-associated genes DCC, EPHB1, SEMASA, and SLIT3 predicated the onset of Parkinson's disease (Maraganore et al., 2005). The authors proposed that after their role during axon guidance, these genes play critical roles in regulating synaptic maintenance (Lin et al., 2009; Maraganore et al., 2005), although an alternative explanation could be that wiring abnormalities made during development as a result of these mutations predispose certain circuits to selective degeneration at later ages.

While GWAS has been useful in identifying low risk alleles for common diseases, it unfortunately does not explain a large proportion of the heritability of complex diseases (Manolio et al., 2009). With the costs of whole-exome sequencing (protein-coding DNA sequences only) and whole-genome sequencing decreasing drastically over the past few years, the polygenic nature of several mental illness and neurological disorders has been uncovered, including schizophrenia (Fromer et al., 2014; Purcell et al., 2014), bipolar disorder (Georgi et al., 2014; Xiao et al., 2014), the autism spectrum disorders (ASD) (Iossifov et al., 2012; Neale et al., 2012; Sanders et al., 2012), and intellectual disability (Girard et al., 2011; de Ligt et al., 2012). For example, high-throughput DNA sequencing analysis of schizophrenic patients revealed that disease liability might be attributed to mutations in approximately one thousand genes (Purcell et al., 2014; Ripke et al., 2013). Computational approaches constructing gene interaction networks identified that the majority of these genes encode proteins involved in axon guidance, synapse formation, and synapse function (Fromer et al., 2014). Interestingly, there was substantial overlap of these genes with those identified using whole-genome sequencing of ASD and intellectual disability (Fromer et al., 2014). Furthermore, it is estimated that this common set of genes is associated with approximately 20-45% of all neurodevelopmental disorders (Krumm et al., 2014), suggesting common wiring dysfunction across several previously unlinked mental illness disorders.

Prior to these large-scale DNA sequencing investigations, it is also worth noting that relatively few human disorders had been attributed to axon guidance defects, although this was primarily due to limited high-resolution imaging technologies to characterize brain disorders at an axonal level (Engle, 2010). Recent advances in high-resolution MRI and diffusion tensor imaging (DTI) have allowed for detailed visualization of fiber tract anatomy, identifying aberrant projections in a number of brain disorders. For example, genetic sequencing and pedigree analysis of patients with ocular motility disorders identified eight heterozygous missense mutations in β-tubulin isotype III (TUBB3) (Tischfield et al., 2010). High-resolution MRI revealed a number of axon connectivity defects, including dysgenesis of the corpus callosum, anterior commissure, and corticospinal tract (Tischfield et al., 2010). Then, by generating a knock-in disease mouse model, it was identified that the TUBB3 mutations have dramatic effects on the ability of kinesin motor proteins to traffic molecular cargo to axons (Tischfield et al., 2010). In another study, genetic analysis of patients with horizontal gaze palsy with progressive scoliosis (HGPPS) identified autosomal recessive mutations in the ROBO3 gene (Jen et al., 2004). By performing high-resolution MRI imaging and evoked-potential electrophysiology, the authors demonstrated the essential role of Robo3 for axon midline crossing (Jen et al., 2004), unlike the role of other Robo family members that mediate repulsive axon guidance to prevent re-crossing.

The development of high-throughput DNA sequencing and computational approaches are allowing for better understanding of how biological processes are defined at a molecular level, of how network disruptions at the systems level lead to disease, and hopefully will lead to therapeutic interventions for improving disease outcomes (Chen et al., 2008; Schadt, 2009; Schadt and Björkegren, 2012). The ability to combine high-throughput genetic sequencing with phenotypic imaging characterization has exciting promise for identifying pathologic molecular and biochemical pathways that cause mental illness and neurological disorders.

In addition to understanding genetic mutations and networks that are associated with mental illness and neurological disorders, understanding neural circuit wiring diagrams will be critical for understanding brain function and identifying abnormalities. However, with the sole exception of the connectivity between the 302 neurons in C. elegans (White et al., 1986), no other organism has had its ‘connectome’ described. Given the enormity of connections in the brain, there are many levels at which connectivity can be described: macro-, meso-, and microscale. Lichtman and colleagues are seeking to generate comprehensive microscale maps of connectivity at the synaptic level using electron microscopy (Lichtman et al., 2014; Morgan and Lichtman, 2013), although current technical limitations have hindered its use for characterizing volumes larger than 1 mm³. At the other end of the scale spectrum, macroscale MRI and DTI imaging have been informative in describing the organization of white-matter tracts, but cellular-level resolution is lost, and thus these analyses are not informative for neural circuit function at the cellular level. Recently, novel technologies are being developed and applied to intricately map brain connectivity at the mesoscale with cellular-level resolution (Oh et al., 2014). In addition to mapping the circuits responsible for neural functions, these efforts also have a long-term goal of promoting our understanding of how neural circuit organization can influence the onset, expression, and course of brain disorders (Fornito et al., 2015; Insel et al., 2013).

It is anticipated that by combining insights from large-scale genetic sequencing with insights from comprehensive, mesoscale brain mapping, a better understanding of genetic susceptibility for particular mental illness and neurological disorders may be achieved, thereby advancing our understanding of the connectivity features of normal and pathologic brains. However, these approaches alone do not shed much insight into how circuits are wired during development, especially since they neglect the molecular networks downstream of genetic programs that actually implement circuit wiring. While computational analysis can predict the interaction networks affected by mutations, this analysis is devoid of subcellular localization and spatiotemporal variability information.

To enable understanding of the networks that control the wiring of specific circuits, the present inventors demonstrate herein the application of high-throughput molecular profiling to specific growth cone populations isolated in vivo. Such understanding enables the elucidation of perturbed molecular networks that underlie brain disorders, and the identification of candidate drug targets for therapeutic intervention. In addition, by systematically characterizing growth cone molecular composition, a robust biomarker system may be obtained to validate potential drugs that are developed to ameliorate wiring abnormalities due to genetic mutations or pathologic networks.

To enable understanding of the networks that control the wiring of specific circuits, disclosed herein is the application of high-throughput molecular profiling to specific growth cone populations isolated in vivo. As more particularly described in the following Examples, by focusing on CPN, general patterns of RNA and protein subcellular localization are uncovered that might control the wiring of the brain circuits integrating information across the two hemispheres. In particular, disclosed herein are a select subset of coding and noncoding RNA transcripts that are specifically enriched in CPN growth cones, suggesting active localization and potential function for navigating axons and forming synaptic connections. As more particularly disclosed in the following Examples, by performing comparative proteomics on CPN cell bodies and growth cones, the present inventors have identified that CPN growth cones have distinct ribosomes from their parent cell bodies. These ribosomes lack several large subunit proteins, raising the intriguing possibility of growth cone-specific translational mechanisms for selective mRNA expression. Taken together, the insights derived from this approach exemplify its capacity to quantify and characterize the molecular and translational mechanisms that control specific brain wiring at the subcellular level in vivo.

While certain disclosures made herein focus on CPN, the approaches described herein and in the accompanying Examples are adaptable to other projection types in the brain, enabling high-throughput, quantitative investigation of RNA and protein controls over circuit development and, potentially, the regeneration of damaged circuitry. By elucidating the molecular networks that wire normal neural circuits, this understanding enables the elucidation of perturbed molecular networks that underlie brain disorders, and allows for the identification of candidate drug targets for therapeutic intervention. By systematically characterizing growth cone molecular composition, we may obtain a robust biomarker system to validate potential drugs that are developed to ameliorate wiring abnormalities due to genetic mutations or pathologic networks. Furthermore, the methods, assays and compositions disclosed herein are scalable to include epigenetic profiling, enabling full investigation of DNA, RNA, and protein networks that collectively implement brain wiring during development.

The teachings set forth herein may be used to promote or otherwise direct axon guidance and thereby treat and/or ameliorate one or more neurologic conditions. For example, in certain embodiments, axons, and in particular axon growth cones may be contacted with one or more guidance cues or canonical cues to direct guidance and, in certain aspects restore or create neural circuits. In some embodiments, such guidance cues or canonical cues comprise one or more of the Netrins, Slits, Semaphorins, and Ephrins.

As used herein, the term “contact” means that two or more substances (e.g., a guidance cue and an axonal growth cone) are sufficiently close to each other such that the two or more substances interact or react (e.g., chemically or biologically) with one another.

In certain aspects, the methods disclosed herein contemplate that administration of one or more agents to the subject for the treatment of a neurologic condition or disease affecting the subject. As used herein, the term “agent” generally refers to any therapeutic (e.g., any compound, nucleic acid, hormone or biological factor) that may be administered to a subject to treat or otherwise ameliorate a neurologic condition affecting such subject. In some embodiments, the agent guides or otherwise directs axon elongation. In certain aspects, an agent is capable of reestablishing a neuronal pathway. In certain aspects, an agent is capable of forming a neuronal pathway. In certain embodiments, the agent targets at least one marker or biomarker that is selectively expressed by growth cones (e.g., a cell surface marker selectively expressed on growth cones). In certain aspects, the agent is selected from the group consisting of small organic molecules, oligosaccharides, polysaccharides, peptides, proteins, peptide analogs, miRNA, siRNA, antisense RNA, and any combination thereof. In still other embodiments, such agents may comprise one or more of the Netrins, Slits, Semaphorins, and Ephrins.

In some embodiments, the agents disclosed herein preferentially target neuron growth cones. For example, such agents may bind to one or more markers or proteins expressed by neuron growth cones and that are not expressed by the neuron body. As used herein, the term “target” means a molecular or biological target structures (e.g., a cell surface receptor) which an agent is able to bind. In certain aspects, the target is or comprises the growth cone GAP-43 marker.

The methods of preparing the compositions and agents of the present invention and selection of pharmaceutically acceptable carriers and excipients are described in detail in, for example, L. William, Remington: The Science and Practice of Pharmacy. 22^{nd} ed. Pharmaceutical Press (2012), the entire contents of which are incorporated herein by reference. It should be noted that the compositions disclosed herein may be administered to a subject via any suitable route of administration, including one or more of the intrathecal, transdermal, buccal, sublingual, enteral or parenteral routes of administration. In certain embodiments, such compositions (e.g., pharmaceutical compositions) may be administered to a subject intrathecally. In certain other embodiments, such pharmaceutical compositions are administered to a subject intravenously.

The compositions, methods and assays disclosed herein may be used, for example, to diagnose, monitor, treat and/or cure the presence or progression of a neurologic condition in a subject. As used herein, a “subject” means a human or animal. In certain embodiments, the subject is a mammal (e.g., a human, non-human primate, mouse, rat, dog, cat, horse, or cow). In certain embodiments, the subject is an adolescent. In certain embodiments, the subject is treated in utero. In certain aspects, a subject in need of treatment in accordance with the methods disclosed herein has a condition or is suspected or at increased risk of developing such condition.

As used herein the phrase “neurologic condition,” is intended to broadly apply to any condition involving or affecting the central nervous system, including for example, any neurodegenerative, neuropsychiatric or neurodevelopmental disorders. In certain aspects the neurodegenerative disorder is selected from the group consisting of Huntington's disease, dentatorubropallidoluysian atrophy, Kennedy's disease, spinocerebellar ataxia, fragile X syndrome, fragile XE mental retardation, Friedreich's ataxia, myotonic dystrophy, spinocerebellar ataxia type 8, spinocerebellar ataxia type 12, Alexander disease, Alper's disease, Alzheimer disease; amyotrophic lateral sclerosis, ataxia telangiectasia, Batten disease, Canavan disease, Cockayne syndrome, corticobasal degeneration, Creutzfeldt-Jakob disease, ischemia stroke, Krabbe disease, Lewy body dementia, multiple sclerosis, multiple system atrophy, Parkinson's disease, Pelizaeus-Merzbacher disease, Pick's disease, primary lateral sclerosis, Refsum's disease, Sandhoff disease, Schilder's disease, spinal cord injury; spinal muscular atrophy, Steele Richardson-Olszewski disease, and Tabes dorsalis. In certain aspects, the neurologic condition is selected from the group consisting of an autism spectrum disorder, schizophrenia, bipolar disorder, and Rett syndrome.

Also disclosed herein are methods of assessing a neurologic condition in a subject based upon the expression of nucleic acids. Such methods comprise a step of identifying differential expression of one or more nucleic acids enriched in neuron growth cones of the subject relative to the expression of the same one or more nucleic acids in neuron growth cones of a normal subject, wherein if the one or more nucleic acids are differentially expressed, the subject has or is at risk of developing the neurologic condition. Similarly, in certain embodiments, the inventions disclosed here relate to methods of monitoring the progression or amelioration of a neurologic condition in a subject, such methods comprising a step of identifying differential expression of one or more nucleic acids in neuron growth cones of the subject relative to the expression of the same one or more nucleic acids in neuron growth cones of a normal subject, wherein if the one or more nucleic acids are differentially expressed, the subject has or is at risk of developing the neurologic condition, and monitoring the expression of the one or more nucleic acids over time, wherein alteration of the expression of the one or more nucleic acids over time is indicative of progression or amelioration of the condition. In some embodiments, the nucleic acid is selected from the group consisting of α-Tub, β-Tub, γ Actin, Basp1, Crmp2, MAP1B, Ncam, Dynein, Basp1, L1cam, Contactin, Gprin1, Stxbp1, Dyncih1, Syn1, Cxadr, Gpm6a, Stx1b, Psmd1 and Mapre1.

It is to be understood that the inventions disclosed herein are not limited in its application to the details set forth in the description or as exemplified. The invention encompasses other embodiments and is capable of being practiced or carried out in various ways. Also, it is to be understood that the phraseology and terminology employed herein is for the purpose of description and should not be regarded as limiting.

While certain compositions, methods and assays of the present invention have been described with specificity in accordance with certain embodiments, the following examples serve only to illustrate the methods and compositions of the invention and are not intended to limit the same.

The articles “a” and “an” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or the entire group members are present in, employed in, or otherwise relevant to a given product or process. Furthermore, it is to be understood that the invention encompasses all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. Where elements are presented as lists, (e.g., in Markush group or similar format) it is to be understood that each subgroup of the elements is also disclosed, and any element(s) can be removed from the group. It should be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in so many words herein. It should also be understood that any embodiment or aspect of the invention can be explicitly excluded from the claims, regardless of whether the specific exclusion is recited in the specification. The publications and other reference materials referenced herein to describe the background of the invention and to provide additional detail regarding its practice are hereby incorporated by reference.

### EXAMPLES

The following studies provide insight on how specific projection neurons wire neural circuits during development, focusing on callosal projection neurons (CPN), which connect the two cerebral hemispheres through the corpus callosum. Also disclosed are novel approaches to profile the growth cone molecular networks that control the wiring of specific neural circuits. The present inventors first describe the development of a methods used to purify projection-specific growth cones by combining in vivo labeling of desired growth cone populations, biochemical fractionation to isolate growth cones, and fluorescent small particle sorting. Also disclosed are the characterization of the full-depth transcriptomes and proteomes of CPN growth cones and their parent cell bodies isolated in vivo. Through comparative analysis, the present inventors uncovered general patterns of RNA and protein subcellular localization, with several previously unrecognized features, that might control the wiring of specific brain circuits. Interestingly, while most transcripts were found to be expressed at similar levels in cell bodies and growth cones, a select subset were more than 10-fold enriched in growth cones compared to cell bodies, suggesting active localization of those transcripts to the growth cone. Also disclosed are studies concerning growth cone-cell body subcellular profiling to investigate protein translation features in growth cones. By first correlating transcriptomic and proteomic data, the present inventors characterized the spatial relationship between coding transcripts and their encoded proteins. Intriguingly, many growth cone-enriched transcripts are noncoding RNA with unknown function. Then, by proteomic analysis, the present inventors found that growth cones appear to have distinct ribosomes. These ribosomes lack several large subunit proteins, raising the intriguing possibility of growth cone-specific translational mechanisms for selective mRNA expression. By then expressing HA-tagged ribosomal protein S25 in growth cones, the present inventors identified translated mRNA transcripts in CPN growth cones.

Precisely defining the molecular networks that control the wiring of specific neural circuits remains a challenge. Although great progress has been made in elucidating axon guidance cues and their growth cone receptors, we still lack an understanding of the projection-specific RNA and protein networks in growth cones that likely control the wiring of specific circuits in vivo. Here, the present inventors developed methods to purify growth cones from specific projections in the brain by combining in vivo labeling, subcellular fractionation, and fluorescent small particle sorting. These methods were applied to quantitatively profile the proteomes and transcriptomes of growth cones and their parent cell bodies from callosal projection neurons, which connect the two cerebral hemispheres through the corpus callosum. Through comparative analysis, the present inventors identified specific RNA and protein networks involved in callosal circuit formation, and identify core growth cone proteomic machinery. This approach is adaptable to any projection in the brain, providing insight into the molecular networks that control the wiring of specific neural circuits in vivo.

The complex functions performed by the brain emerge from intricate wiring connections formed between distinct brain regions during development. The mammalian neocortex, the region of the brain primarily responsible for cognitive function, sensory perception, and consciousness, connects with specific targets throughout the nervous system to achieve its function (Finlay and Darlington, 1995; Molyneaux et al., 2007). With enormous complexity in neocortical connectivity, it is not surprising that many neurological disorders have been attributed to connectivity abnormalities, including the autism spectrum disorders (Barttfeld et al., 2011; Geschwind and Levitt, 2007; Minshew et al., 2007), schizophrenia (Avesar and Gulledge, 2012; Innocenti et al., 2003), and Rett syndrome (Kishi and Macklis, 2010; Sceniak et al., 2015). By characterizing the molecular networks that wire neocortical circuits during development, insight may be gained into the pathologic molecules and/or molecular networks that underlie these disorders.

Despite the enormity of connections and complexity in the neocortex, there is a high level of organization in the circuits connecting distinct brain regions (Tau and Peterson, 2010). This organization is largely controlled during development by growth cones, dedicated subcellular structures located at the distal tip of extending axons. Growth cones guide axons by the expression of specific transmembrane receptors and intracellular signaling machinery that collectively detect and interpret molecular guidance cues presented in the extracellular environment (Dickson, 2002; Lowery and Van Vactor, 2009; Tessier-Lavigne and Goodman, 1996). After reaching their final targets, growth cones transform molecularly and morphologically into presynaptic terminals, and become specialized for chemical neurotransmission.

Throughout most of their trajectory, growth cones are located substantial distances away from their parent cell bodies. By severing axons from their parent cell bodies, it has been shown that growth cones can survive and make turning decisions for a few hours (Harris et al., 1987; Shaw and Bray, 1977), demonstrating that growth cones have a degree of autonomy from cell bodies, and that they contain molecular machinery required for making local responses to extracellular cues (Shigeoka et al., 2013). To understand the molecular machinery that underlies growth cone autonomy and their ability to wire neural circuits, recent work has started to characterize growth cone transcriptomes (Gumy et al., 2011; Minis et al., 2014; Taylor et al., 2009; Zivraj et al., 2010) and proteomes (Estrada-Bernel et al., 2012; Nozumi et al., 2009). However, these studies have been limited to investigating growth cones in vitro, where specificity for forming neural circuits is lost, or have analyzed heterogeneous growth cone populations isolated from the entire brain, where insight into the formation of specific neural circuits is not possible. Devising in vivo approaches to profile growth cones from distinct neuron subtypes might contribute substantially to our understanding of how specific neural circuits are wired during development.

The present inventors devised an approach to elucidate the growth cone molecular networks that control the wiring of specific neural circuits. After first demonstrating that axons have intrinsic mechanisms for wiring neural circuits, described herein are methods of purify projection-specific growth cones by combining in vivo labeling of desired neuron populations, subcellular fractionation, and newly-developed fluorescent small particle sorting. This method was applied to profile growth cones from CPN, which connect the two cerebral hemispheres through the corpus callosum. CPN are a clinically important neuron population, as abnormalities in their development and connectivity have been linked to several mental illness and neurological disorders (Fame et al., 2011). The full-depth transcriptomes and proteomes of CPN growth cones and their parent cell bodies isolated in vivo are then elucidated. Through comparative analysis, the present inventors uncovered patterns of RNA subcellular localization, with several previously unrecognized features, that might control the wiring of specific brain circuits.

### Example 1

### Axons have Intrinsic Mechanisms for Wiring Neural Circuits

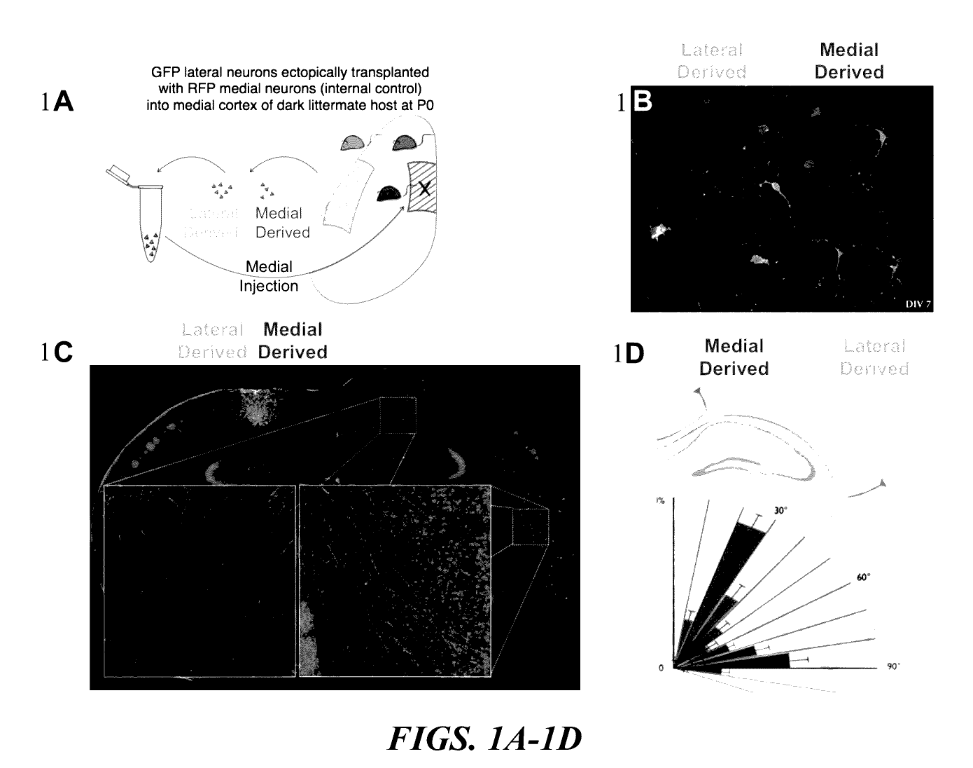

To first directly investigate whether axons have intrinsic mechanisms for wiring neural circuits, the present inventors performed heterotopic and homotopic micro-transplantation of neuron cell bodies and analyzed their axonal trajectories. We crossed heterozygous TdTomato with heterozygous GFP mice, generating littermates that were TdTomato+; GFP+; TdTomato+, GFP+; or dark (TdTomato−, GFP−). At postnatal day 0 (P0), neurons were isolated from the medial cortex of TdTomato+ mice, and from the lateral cortex of GFP+ mice. Mixing these populations together into a single-cell suspension, the present inventors transplanted them into the medial cortex of dark littermates (FIG. 1A). With TdTomato+ medial-derived neurons serving as an internal transplantation control, we find that the vast majority of transplanted TdTomato+ axons project toward their natural targets medially in the contralateral cortex (FIGS. 1C and 1D). In contrast, the vast majority of GFP+ lateral-derived axons project toward their natural targets in the lateral contralateral cortex, despite being heterotopically transplanted into medial cortex (FIGS. 1C and 1D). These results indicate that CPN axons have the intrinsic knowledge of where to project in vivo, suggesting that mechanisms within axons determine wiring decisions.

### Example 2

### Isolating Growth Cones from the Developing Brain

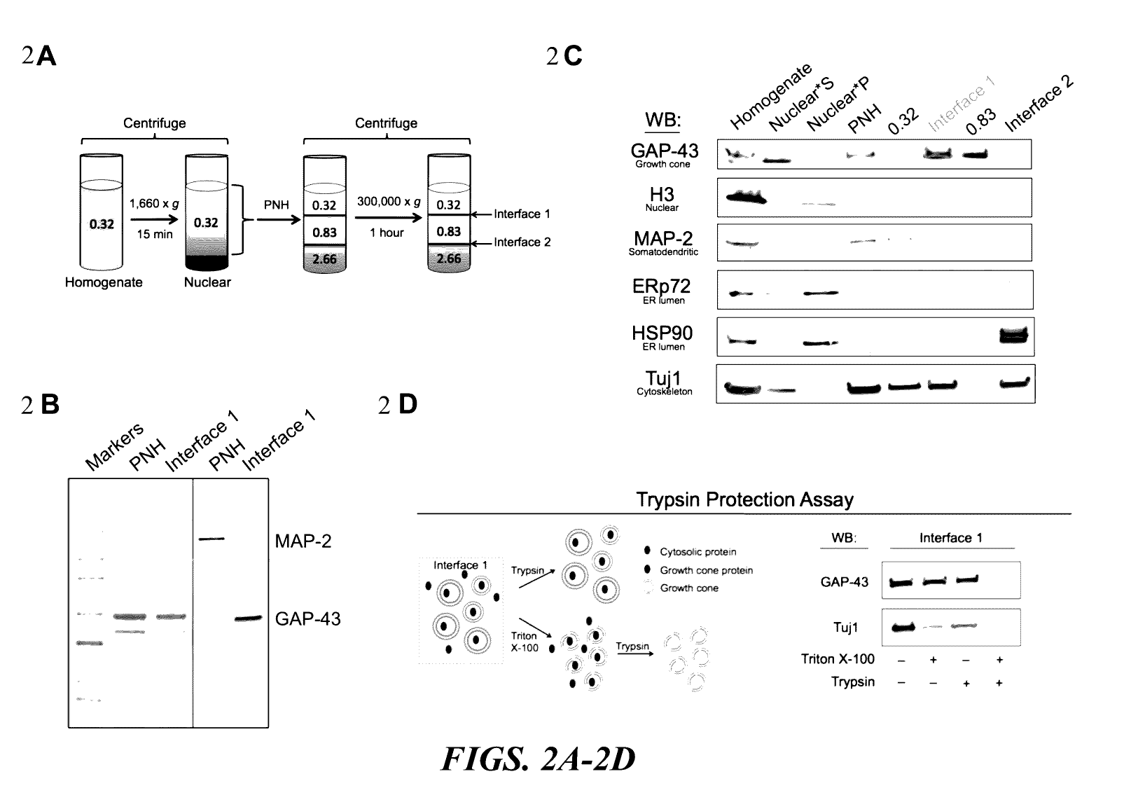

It has previously been shown that an enriched population of growth cones can be isolated using isopycnic subcellular fractionation (FIG. 2A) (Lohse et al., 1996; Nozumi et al., 2009; Pfenninger et al., 1983). The present inventors analyzed the purity of growth cone fractionation by collecting distinct fractions and probing for subcellular marker proteins using Western blot. The present inventors found that the growth cone fraction, extracted at the interface of two sucrose solutions of different density, is enriched in GAP-43, an established growth cone marker protein (Meiri and Gordon-Weeks, 1990), compared to input postnuclear homogenate (PNH) (FIGS. 2B and 2C). In contrast, there was no detection of markers from other subcellular structures in the growth cone fraction, including nuclei, dendrites, Golgi and rough ER (FIG. 2C). Notably, these structures collect at a distinct interface of higher density (FIG. 2C), and thus are not minor fractions within the growth cone fraction.

To distinguish between membranous growth cone proteins and cytosolic proteins that are known to co-fractionate with the growth cone fraction (Pfenninger et al., 1983), membrane integrity was assayed by treatment with combinations of detergent and trypsin (FIG. 2D). The present inventors find that treatment with Triton X-100 or trypsin alone moderately degrades β-tubulin levels, but has no effect on GAP-43 levels (FIG. 2D). Treatment with Triton X-100 to disrupt membrane integrity prior to trypsin treatment leads to complete degradation of GAP-43 and β-tubulin (FIG. 2D). Since GAP-43 levels are not affected by trypsin treatment, this result indicates that isolated growth cones have intact membranes. In addition, while some soluble proteins like β-tubulin are present in the growth cone fraction, they can be selectively removed by protease treatment, or by passage through cell sorter fluidic systems.

The present inventors next analyzed the distribution of growth cone RNA using Bioanalyzer analysis. Similar to RNA isolated from cell bodies, growth cone RNA is comprised predominantly of ribosomal RNA (rRNA), and notably is of high quality (RIN>8) (FIG. 2.3A). RNase protection was also assayed, finding that RNase treatment without detergent has no effect on RNA levels (FIG. 2.3B). In contrast, treatment with Triton X-100 prior to RNase treatment leads to total degradation of RNA, indicating that RNA extracted from the growth cone fraction is encapsulated within growth cones, and is not contaminated with cytosolic-derived RNA.

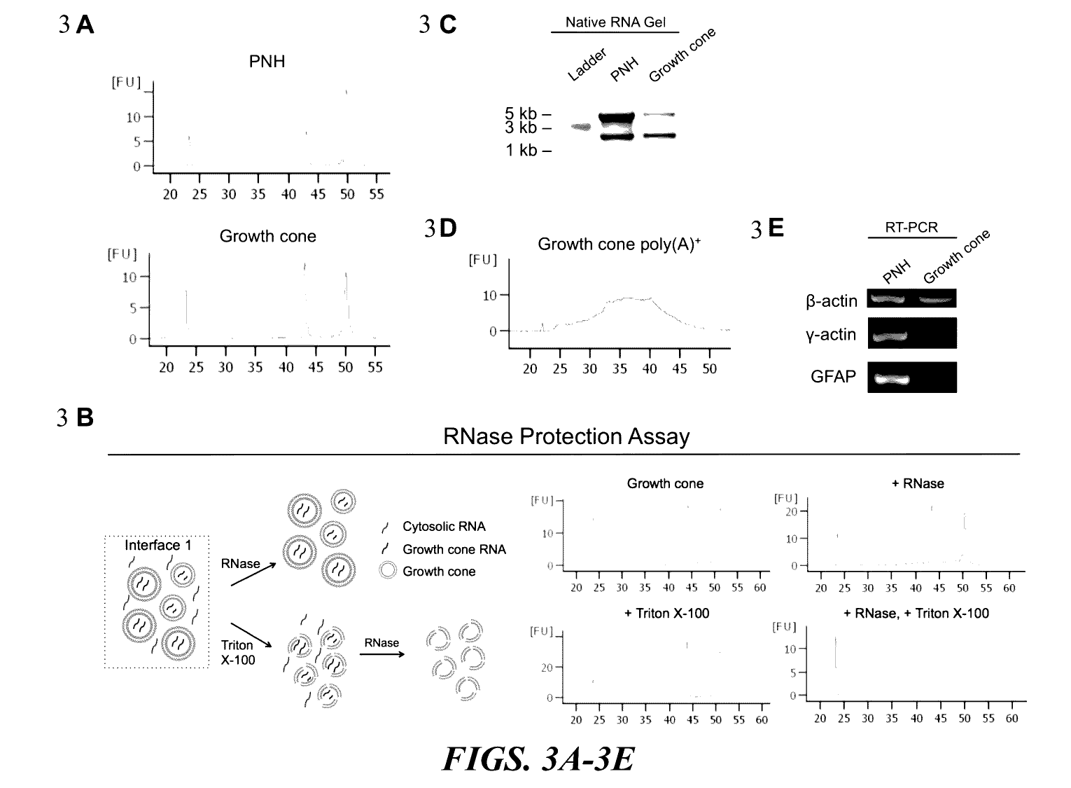

By isolating mRNA through poly(A) selection, a wide distribution of mRNA transcripts was found in the growth cone fraction (FIG. 3D). It has previously been shown that two actin isoforms have differential expression in neurons; γ-actin mRNA is restricted to the cell body, while P-actin mRNA is present at high levels in growth cones in addition to presence in the cell body (Bassell et al., 1998). Using RT-PCR, β-actin mRNA was found to be present in growth cones, but that γ-actin mRNA was excluded (FIG. 3E), consistent with previous in vitro studies.

Taken together, these results indicate that a pure population of membrane-intact growth cones can be isolated from the developing brain, and are devoid of other membrane-bound subcellular structures.

### Example 3

### Purifying Fluorescently Labeled Growth Cone Populations

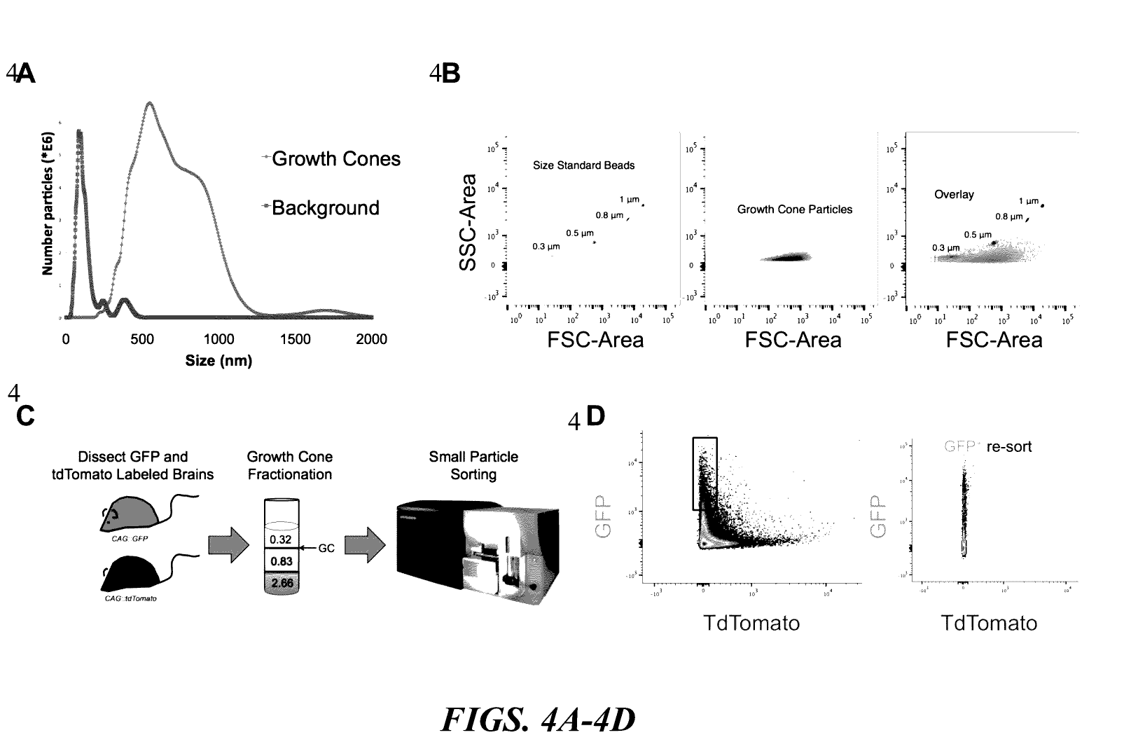

Recent advances in high-resolution flow cytometry have allowed for the characterization and purification of nanosized membrane vesicles with diameters as small as 300 nm (Nolte-'t Hoen et al., 2012; van der Vlist et al., 2012). Using nanoparticle-tracking analysis (NTA) (Filipe et al., 2010), the present inventors found that growth cones isolated in vivo range between 300-1,000 nm in diameter (FIG. 4A). Since isolated growth cones have intact membranes (FIGS. 2D and 3B) and are in the size range for high-resolution flow cytometry, the present inventors devised an approach to purify labeled growth cone populations for investigation of the intrinsic molecular mechanisms that wire specific circuits.

For characterizing and sorting fluorescently labeled growth cones, the present inventors used a customized Special Order FACS Aria III cell sorter equipped with high-performance photomultiplier tubes for forward scatter measurement, which enables sensitive detection of fluorescent small particles. The present inventors first analyzed the size distribution of growth cones mixed with size-standard beads, finding that growth cones range between 300-1,000 nm in diameter (FIG. 4B), consistent with our NTA measurements. Next, the present inventors evaluated the ability to discriminate between distinct growth cone populations labeled differentially with fluorescent proteins (FIG. 4C). The present inventors isolated growth cones from GFP+ mice and TdTomato+ mice and mixed the two populations together. EGFP+ growth cones were readily resolved from TdTomato+ growth cones, with no evidence of aggregation (FIG. 4D). In addition, the ability to successfully purify EGFP+ growth cones after detection by subjecting sorted EGFP+ growth cones to a subsequent resort was confirmed (FIG. 4D).

### Example 4

### Purifying Circuit-Specific Growth Cone Populations

To specifically label CPN axons and growth cones, the present inventors found that the brightest fluorescent labeling is achieved using membrane-anchored fluorescent proteins, although cytosolic fluorescent proteins can also be used with success. To analyze appropriate time points for CPN growth cone isolation, the present inventors used in utero electroporation to express a CAG>myristoylated (myr)-TdTomato expression plasmid into ventricular zone progenitors at E15.5, which is the peak of neurogenesis for layer II/III CPN (Molyneaux et al., 2007). By characterizing CPN axon position at several postnatal ages, the present inventors found that between P2-P3, CPN axons from the lateral neocortex cross the midline through the corpus callosum. By P7, CPN axons have turned into the grey matter of the contralateral cortex into their synaptic target fields (data not shown).

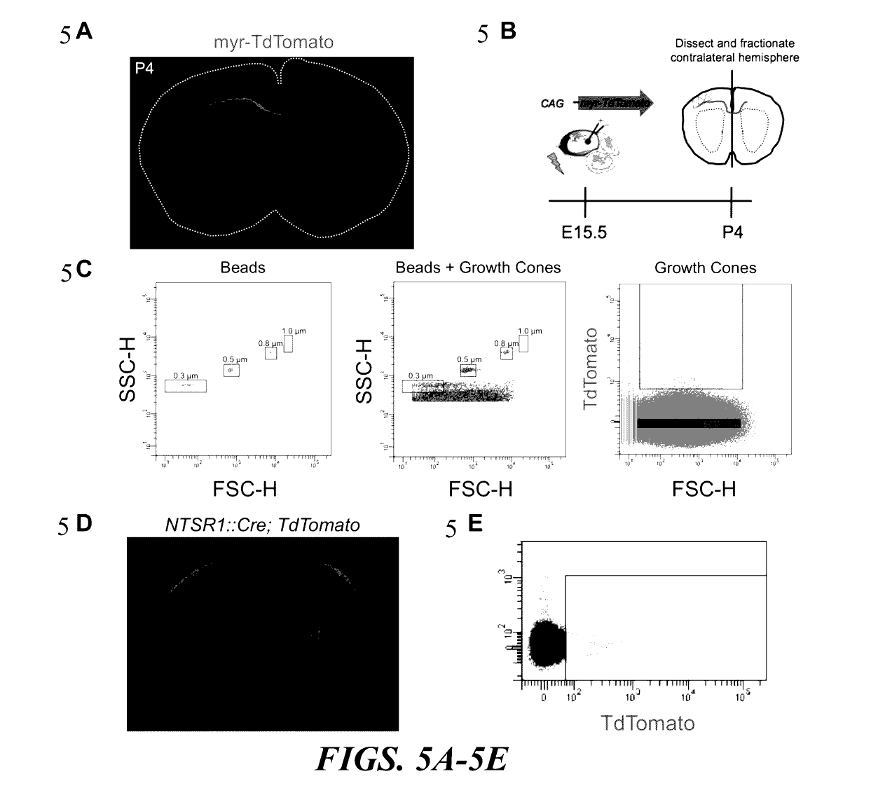

From this characterization, the present inventors focused analyses on investigating growth cone molecular networks at P4, when CPN axons are actively navigating along white matter tracts (FIG. 5A). After using in utero electroporation to unilaterally label CPN progenitors at E15.5 with myr-TdTomato, the present inventors selectively dissected the contralateral hemisphere for growth cone isolation using subcellular fractionation (FIG. 5B). The present inventors then confirmed the ability to sort TdTomato+CPN growth cones using this approach (FIG. 5C).

In order to dissect the complexity and function of neural circuits, several genetically engineered mouse lines have been generated to conditionally label specific neuron subtypes of interest with fluorescent proteins, or to express other genetically targeted payloads such as Channelrhodopsins (Huang and Zeng, 2013; Madisen et al., 2015). One of the most widely used genetic strategies is the Cre/loxP binary system, and over 100 Cre driver lines have been developed by the Allen Institute's Transgenic Characterization initiative alone (Harris et al., 2014; Madisen et al., 2015). While the present inventors have primarily used in utero electroporation to label CPN, this approach can also readily be combined with Cre driver mouse lines to selectively label desired other neuron subtypes for subsequent isolation of their growth cones that wire a neural circuit of interest. To demonstrate the successful utilization of Cre lines to label growth cones, the present inventors crossed the NTSR::Cre mouse line (Gong et al., 2007), which selectively labels corticothalamic projection neurons (CThPN) (Galazo et al., unpublished), with a Rosa26^{flax-STOP-TdTomato} reporter mouse line (Madisen et al., 2010) (FIG. 5D). The present inventors found that this strategy enables purification of TdTomato+CThPN growth cones (FIG. 5E).

Taken together, these results indicate that specific fluorescently labeled growth cone populations can be successfully purified using this newly developed fluorescent small particle sorting approach.

### Example 5

### Elucidating Projection-Specific Growth Cone Proteomes

Recent proteomic and bioinformatic analyses of bulk growth cones isolated from the entire brain have identified a number of growth cone proteins known to function in cell biological processes including axonal pathfinding, cytoskeleton remodeling, vesicular trafficking, protein translation, and the proteasome (Estrada-Bernal et al., 2012; Nozumi et al., 2009). However, as these heterogeneous growth cone populations were derived from likely hundreds of distinct neuron subtypes in the brain all intermixed, the ability to examine molecular networks of proteins in one particular growth cone class that wire specific neural circuits has not previously been possible.

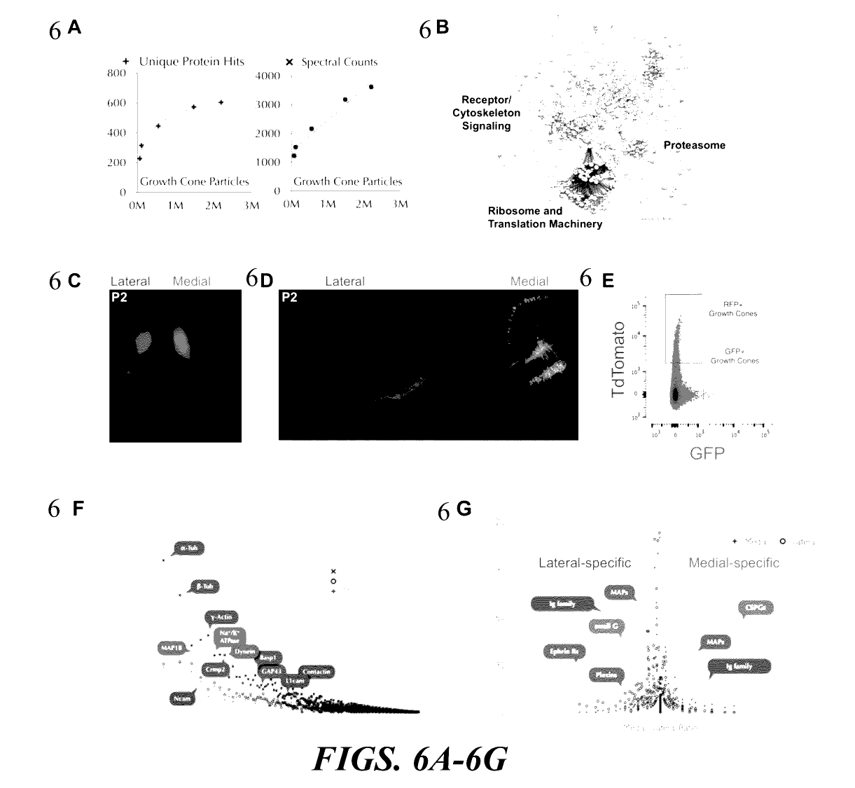

In order to elucidate the full-depth proteome of CPN growth cones, the present inventors first determined the number of sorted growth cones, and therefore the mass of proteins, required for full-depth proteomic characterization and quantification. The present inventors sorted increasing numbers of TdTomato+CPN growth cones, extracted their enclosed proteins, and performed high-resolution liquid chromatography-tandem mass spectroscopy (LC MS/MS). the present inventors found that approximately 1 million growth cones are required for full coverage and linearity over the quantifiable dynamic range (FIG. 6A).

Growth cones likely have (1) general proteomic networks common to all projection neuron growth cones, and (2) circuit-specific proteins that allow for selective response to guidance cues and implementation of synaptic connectivity. To classify the molecular components common to all projection neurons, and distinguish those that are likely to be circuit-specific, the present inventors first analyzed the proteomic differences between medial and lateral CPN growth cones. Medial and lateral CPN are highly related populations at the transcriptional identity level in the nucleus, but their axons project toward distinct homotopic regions of the contralateral cortex. The present inventors performed dual in utero electroporation to label medial CPN with GPI-EGFP and lateral CPN with myr-TdTomato (FIGS. 6C and 6D), and purified medial GFP+ and lateral TdTomato+ growth cones (FIG. 6E).

After identifying proteins using proteomics, the present inventors performed clustering analysis using the STRING database to identify protein interaction networks. Using this analysis, the present inventors found that the most significant molecular networks in both lateral and medial CPN growth cones are receptor/cytoskeleton signaling pathways, ribosome and translation machinery, and the proteasome (FIG. 6B). Notably, medial/lateral-specific differences include members of the Ig-superfamily, CSPGs, and EphrinBs, although these proteins are expressed at significantly lower levels than ubiquitous components (FIGS. 6F and 6G). For comparison with a distinct neuron subtype, performed comparative proteomic analysis between CPN and CThPN growth cones was performed. It was determined that the most highly expressed protein networks are common between these two populations, with projection-specific differences in lower abundance receptors and signaling molecules (data not shown).

Taken together, these comparative proteomic analyses support the hypothesis that growth cones have ubiquitous core machinery, along with projection-specific differences for wiring specific neural circuits.

### Example 6

### Comparative Subcellular RNA-Seq Reveals Highly Enriched Growth Cone Transcripts

While descriptive proteomic or transcriptomic datasets from specific growth cone populations isolated in vivo are informative, the ability to quantify RNA and protein expression levels in growth cones relative to their parent cell bodies would enable investigation of mechanisms that govern subcellular localization. The present inventors therefore devised a subcellular profiling approach to purify growth cones and their parent cell bodies in parallel by taking advantage of the symmetry of CPN, whose cell bodies reside in one hemisphere and project their axons toward homotopic targets in the contralateral hemisphere.

To label cell bodies and their axons, the present inventors used in utero electroporation to introduce a CAG>myr-TdTomato-2A-H2B-EGFP expression plasmid into progenitors at E15.5, thus labeling CPN nuclei with GFP and, as above, their axons with TdTomato (FIGS. 7A and 7B). At P3, the ipsilateral electroporated hemisphere was dissected and dissociated the cortex into single cells for purification of GFP+CPN cell bodies using traditional fluorescence-activated cell sorting (FACS) (FIG. 7C). In addition, as discussed previously, the present inventors isolated growth cones from the contralateral hemisphere, purifying TdTomato+CPN growth cones using fluorescent small particle sorting (FIG. 7D).

From sorted growth cones and their parent cell bodies, the present inventors isolated RNA (FIGS. 7C and 7D), generated independent RNA-seq libraries from equal masses of growth cone or cell body RNA, and sequenced to a mean of 50 million mapped 100 basepair paired-end reads per library. The distribution of transcripts previously shown to be soma-restricted or growth cone-localized as first analyzed. The expression profiles of growth cone-localized β-actin transcripts and soma-restricted Xist and γ-actin transcripts were consistent with the known patterns of expression (FIG. 7E). The distribution of enrichment across all mapped transcripts in growth cones was then analyzed relative to their expression in cell bodies. Unsurprisingly, the present inventors found that a substantial percentage (approximately 30%) of transcripts are restricted to the cell body (FIG. 7F), consistent with the large number of transcripts known to be restricted to the nucleus. While most transcripts are expressed at similar levels in cell bodies and growth cones, a select subset of approximately 425 transcripts are more than 10-fold enriched in growth cones compared to cell bodies (FIG. 7F), suggesting active localization of these transcripts to growth cones. In ongoing work, the present inventors are performing bioinformatics analyses to identify localization motifs (data not shown).

Interestingly, nearly 30% of the highly growth cone-enriched transcripts are long noncoding RNA transcripts (lncRNA) (FIG. 7F), which have not previously been reported to localize to growth cones. Future work investigating the potential regulatory and/or signaling function of these top candidate noncoding RNA transcripts might shed further insight into the complexity of molecular networks that implement neural circuit connectivity during development.

### Example 7

### Growth Cones have Distinct Ribosome Proteins

During development, growth cones locally control axon navigation decisions by dynamically regulating their complement of receptors and intracellular signaling molecules to selectively interpret guidance cues. In addition to regulation at the proteomic level, local translation of specific mRNA transcripts is emerging as an important layer of regulatory control in axons and growth cones. While several mRNA transcripts have been shown to be locally translated in growth cones in vitro, the mechanisms that regulate native growth cone translation remain largely unknown. In the following examples, the present inventors identify that growth cones have distinct ribosomes. By performing comparative subcellular proteomics from a specific neuron subtype, The present inventors found that growth cone ribosomes specifically lack several 60S subunit ribosome proteins, in contrast to the full complement of 60S proteins expressed in ribosomes in their parent cell bodies. By tagging a ribosome protein localized to growth cones, the present inventors identified translated mRNA transcripts in growth cones in vivo. Having a distinct ribosome protein composition in growth cones might be a mechanism to endow growth cones with selective translational control over mRNA expression.

The connectivity of the mammalian brain is implemented during development by the interplay of intrinsic molecular programs that specify cell identity and cell extrinsic factors that communicate spatiotemporal guidance information to navigating axons. Extending axons are led to their targets by specialized subcellular structures known as growth cones, which contain molecular networks comprised of RNA, proteins, and lipids that implement circuit connectivity decisions. Growth cones navigate axons through a complex and heterogeneous developing brain environment by selectively responding to molecular guidance cues, requiring growth cones to have an ability to dynamically regulate their interpretation of cues at key choice points along their trajectories (Dickson, 2002; Kolodkin and Tessier-Lavigne, 2010). This regulation can be achieved by several mechanisms at the proteomic level, including receptor downregulation, ligand-mediated receptor inactivation, and proteolytic degradation (Yu and Bargmann, 2001). Recent in vitro findings have revealed an additional layer of regulation at the RNA level, by which translation of specific mRNA transcripts has been reported in growth cones in response to extrinsic cues in vitro (Campbell and Holt, 2001; Colak et al., 2013; Leung et al., 2006; Leung et al., 2013; Wu et al., 2005; Yao et al., 2006; Yoon et al., 2012).

As highly polarized cells with an asymmetrical distribution of proteins, neurons localize mRNA transcripts to distinct compartments of the cell (Holt and Bullock, 2009). Using RNA-sequencing from growth cone RNA isolated in vitro, several reports have indicated that potentially thousands of mRNA transcripts are localized to axons and growth cones (Gumy et al., 2011; Minis et al., 2014; Taylor et al., 2009; Zivraj et al., 2010). With fast axonal transport able to traffic protein and mRNA transcripts from cell bodies to growth cones at rates of only 0.5-1.0 μm/second (Brady, 1991; Duncan and Goldstein, 2006), local translation endows growth cones with an ability to rapidly and locally modify their protein complement in response to extracellular cues. For example, asymmetric local translation of 3-actin has been reported to occur in growth cones within 5 minutes in response to the chemoattractant molecule Netrin-1 (Holt and Schuman, 2013; Leung et al., 2006; Shestakova et al., 2001). Local translation in growth cones has also been shown to be important for axon pathfinding decisions (Brittis et al., 2002; Leung et al., 2013), axon survival, (Yoon et al., 2012) and axon regeneration (Verma et al., 2005; Zheng et al., 2001). However, since previous reports utilized in vitro culture systems, in vivo mechanisms of local translation in growth cones remain unexplored.

While there is substantial evidence for local translation in growth cones, little is known about the molecular composition of ribosomes in growth cones, and whether they are distinct from ribosomes in their parent cell bodies. Recent reports have revealed that the proteomic composition of ribosomes varies across cell types (Kondrashov et al., 2011; Reschke et al., 2013), and that absence of specific ribosome proteins affects translation of specific mRNA transcripts (Kondrashov et al., 2011; Mazumder et al., 2003). These findings have shifted the classical view that ribosomes are constitutively active in favor of a model in which ribosomes possess intrinsic regulatory ability for selective translation of specific subsets of mRNA transcripts (Xue and Barna, 2012).

In the following studies, the present inventors report that the ribosome composition in growth cones is distinct from the composition in their parent cell bodies. First finding that RpL10a, a 60S subunit protein that is the basis of the translating ribosome affinity purification (TRAP) methodology (Doyle et al., 2008; Heiman et al., 2008), is not expressed in growth cones in the neocortex. In order to investigate growth cone translational mechanisms in a specific cell-type, the present inventors characterize the ribosome composition in growth cones and their parent cell bodies isolated in vivo from callosal projection neurons (CPN), which connect the two cerebral hemispheres through the corpus callosum. Comparing the ribosome protein distribution in CPN growth cones with the distribution in their parent cell bodies, the present inventors found that many 60S subunit proteins are not expressed in growth cones. By then expressing HA-tagged RpS25, a ribosome protein expressed in growth cones, the present inventors perform ribosome affinity purification to identify translated mRNA transcripts in CPN growth cones, providing the first direct in vivo evidence for local translation.

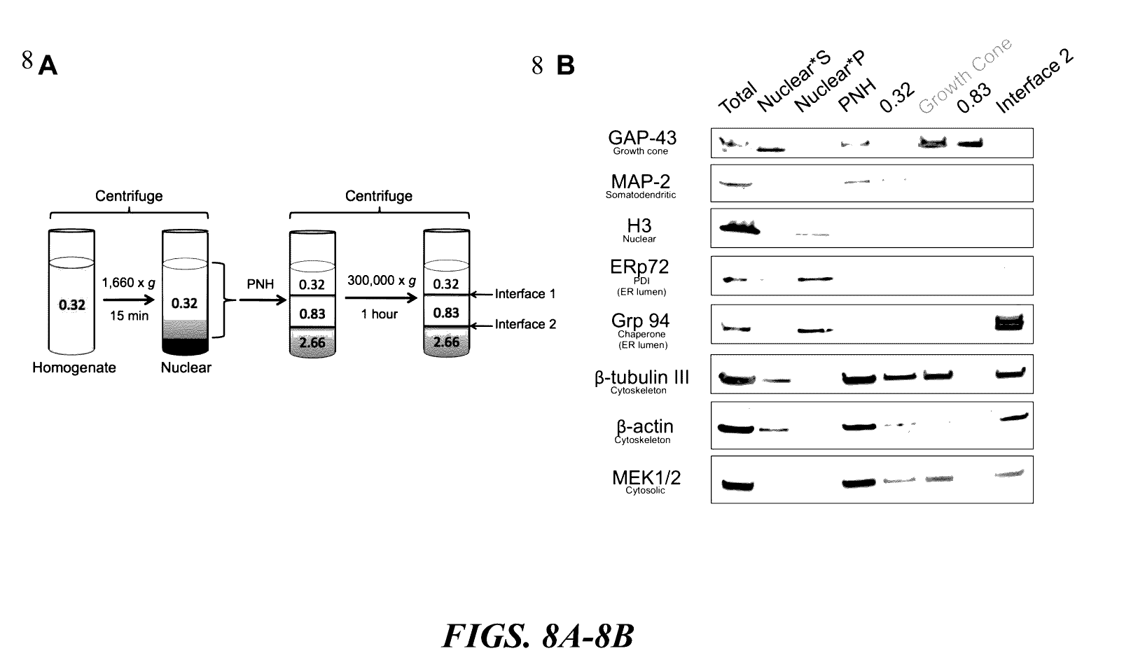

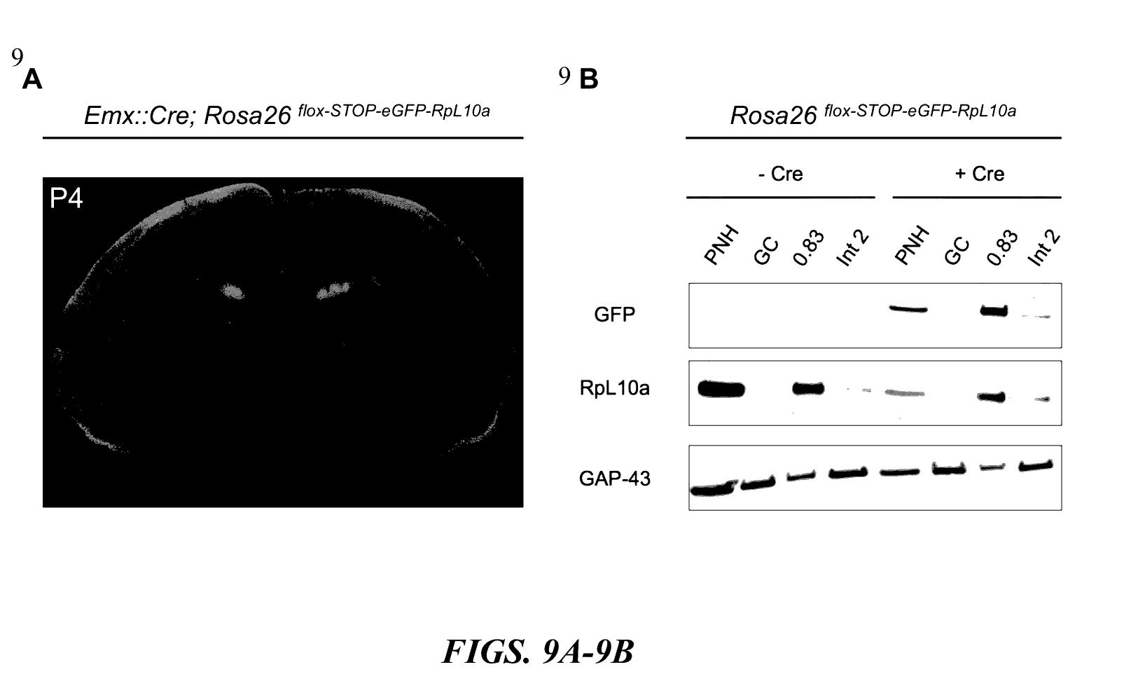

In the previous examples the present inventors describe that a pure population of membrane-intact growth cones can be isolated from the developing brain using isopycnic subcellular fractionation. Importantly, the growth cone fraction, which is extracted at the interface of two sucrose solutions of different density (Lohse et al., 1996; Pfenninger et al., 1983), is devoid of other subcellular structures, including nuclei, dendrites, Golgi, and rough ER (FIGS. 8A and 8B). To investigate local translation in growth cones, the present inventors first examined utilization of the translating ribosome affinity purification (TRAP) methodology, which targets an eGFPRpL10a ribosomal fusion protein (part of the 60S subunit) to desired cell types, thus enabling affinity purification of cell type-specific translated mRNA transcripts (Heiman et al., 2008). To express eGFP-RpL10a in neocortical projection neurons, the present inventors crossed Emx1::Cre+/− and Rosa26^{flax-STOP-eGFP-RpL10a} mice (Liu et al., 2014). After first verifying that eGFP-RpL10a expression is restricted to Cre+ neurons in the neocortex (FIG. 9A), the present inventors isolated neocortical growth cones from Emx1::Cre; Rosa26^{flax-STOP-eGFP-RpL10a} mice at postnatal day 4 (P4), a time when projection neuron axons are actively navigating along white matter tracts. Interestingly, the present inventors found that eGFP-RpL10a is not detected in growth cones by Western blot, in contrast to its strong expression in cell bodies (FIG. 9B). Endogenous RpL10a is also not detected in growth cones (FIG. 9B), collectively indicating that growth cone ribosomes lack RpL10a.

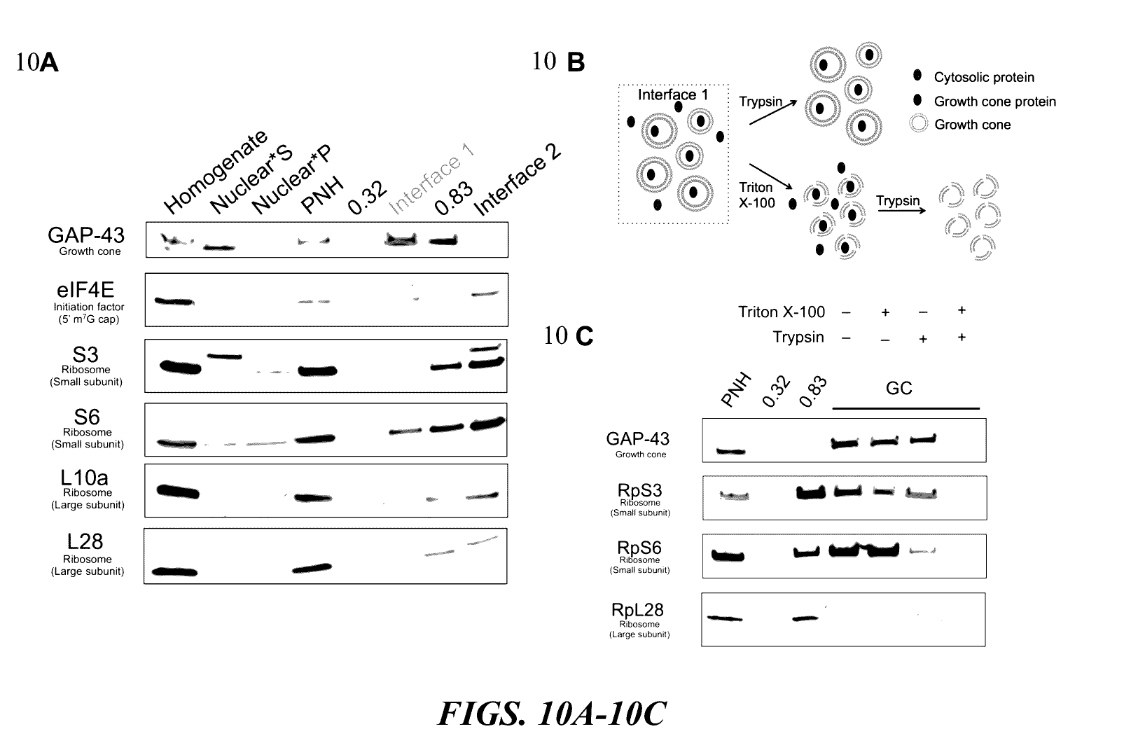

The present inventors next analyzed the expression of additional ribosome proteins in growth cones using Western blot. The present inventors found that the 40S ribosome proteins RpS3 and RpS6, and the translation initiation factor eIF4E are present in growth cones (FIG. 10A). In contrast, in addition to RpL10a being absent, the present inventors found that the 60S ribosome protein RpL28 is absent from growth cones (FIG. 10A). The present inventors then performed a trypsin protection assay to distinguish between ribosome proteins that are enclosed within growth cones, and contaminant ribosomes derived from the cytosol or the outer surface of the ER (FIG. 10B). The present inventors found that trypsin treatment without detergent moderately degrades RpS3 and RpS6 levels, but has no effect on the levels of the growth cone marker GAP-43 (FIG. 10C). In contrast, treatment with Triton X-100 to disrupt membrane integrity prior to trypsin treatment leads to complete degradation of GAP-43, RpS3, and RpS6 (FIG. 10C). These results indicate that, while some cytosolic-derived ribosomes are present in the growth cone fraction, they can be selectively removed by protease treatment, or by passage through cell sorter fluidic systems.

Taken together, these results suggest that growth cones have distinct ribosomes, prompting us to investigate the global composition of ribosome proteins in growth cones, and compare it with the composition of ribosome proteins in their parent cell bodies.

### Example 8

### Comparative Subcellular Proteomics Reveal Distinct Growth Cone Ribosome

In the previous Examples, the present inventors reported that proteomic analyses of sorted CPN growth cones can uncover their full-depth proteomes, including transmembrane receptors, cytoskeletal proteins, and signaling molecules (see, FIG. 6A). By performing clustering analysis on identified growth cone proteins using the STRING database to assemble protein interaction networks, one of the most significant molecular networks in CPN growth cones is the ribosome and translation machinery was identified (see FIG. 6B). The present inventors also identified that the ribosome and translation machinery network is highly significant in corticothalamic PN (CThPN) growth cones. Interestingly, a previous report that performed proteomic and bioinformatic analyses on growth cones isolated from the entire brain also found high enrichment of translational networks in growth cones (Estrada-Bernal et al., 2012), suggesting that translation machinery is common to growth cones from many neuron subtypes.

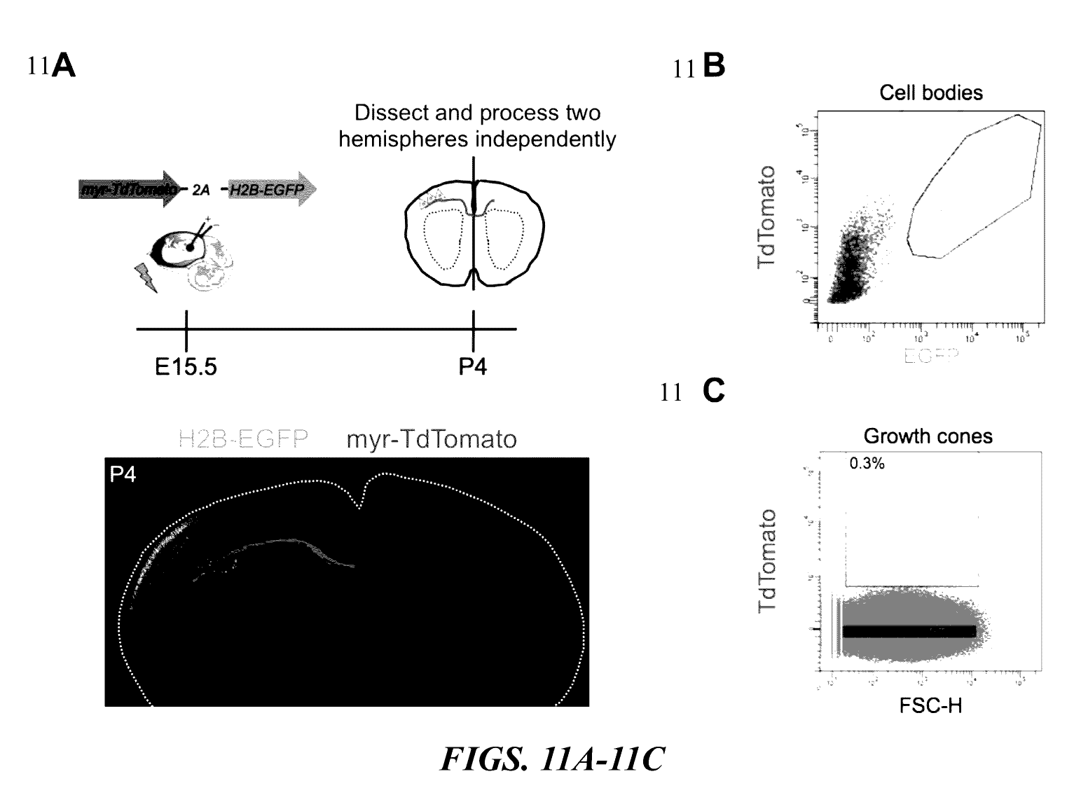

In order to analyze the proteomic composition of growth cone ribosomes and cell body ribosomes, the present inventors utilized a recently developed subcellular profiling approach to profile CPN cell body and growth cone molecular networks. CPN are an advantageous neuron population for investigation of growth cone translation because their cell bodies and growth cones are located in distinct hemispheres after P2 (FIG. 11A), enabling an anatomical purification strategy to independently isolate cell bodies and their growth cones in parallel.

To label CPN cell bodies and their axons, the present inventors used in utero electroporation to introduce a CAG>myr-TdTomato-2A-H2B-eGFP expression plasmid into CPN progenitors at E15.5, thus labeling CPN nuclei with eGFP, and axons with TdTomato (FIG. 11A). At P4, the ipsilateral electroporated hemisphere was dissected, and the cortex dissociated into single cells for purification of eGFP+CPN cell bodies using fluorescence-activated cell sorting (FACS) (FIG. 11B). In addition, the present inventors isolated growth cones from the contralateral hemisphere, and purified TdTomato+CPN growth cones using fluorescent small particle sorting (FIG. 11C).

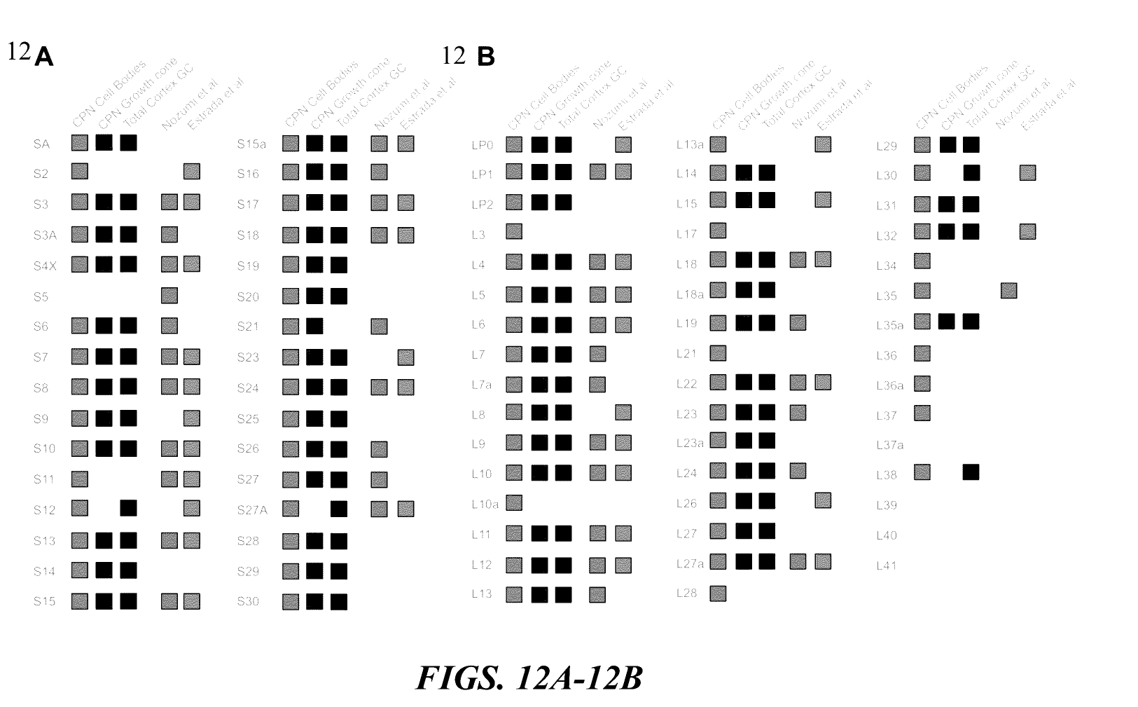

Comparative proteomic analyses were performed to uncover whether the complement of ribosomal proteins in CPN growth cones is distinct from their parent cell bodies. The vast majority (74/79) of the known ribosome proteins in the fully assembled mammalian 80S ribosome (Ben-Sherr et al., 2011; Lecompte et al., 2002) were detected in CPN cell bodies by proteomics (FIGS. 12A and 12B). In contrast, many ribosome proteins were not detected in CPN growth cones, most of which are proteins in the 60S subunit (17/47 60S proteins not detected; 4/32 40S proteins not detected) (FIGS. 12A and 12B). Importantly, RpL10a and RpL28 were not detected, confirming my Western blot findings.

The present inventors next compared the growth cone ribosome protein distribution with two previously described growth cone proteomes assembled from growth cones isolated from the total cortex (Estrada-Bernal et al., 2012; Nozumi et al., 2009). Since these proteomes were derived from likely hundreds of distinct neuron subtypes in the brain all intermixed, and therefore lack subtype-specificity, the present inventors additionally performed proteomic analysis on growth cones isolated from the total cortex for more direct comparisons. For the ribosome proteins detected in growth cones by these previous reports, the present inventors found extensive overlap with my analysis (FIGS. 12A and 12B). Notably, however, the present inventors detected the presence of significantly more ribosome proteins than previously reported.

This discrepancy is likely explained by our utilization of the Orbitrap Elite mass spectrometer for our proteomic analyses (Michalski et al., 2011), which has several-fold higher sensitivity than the mass spectrometers used in previous growth cone proteomic analyses.

Taken together, comparative proteomic analyses support the hypothesis that growth cones have distinct ribosomes from their parent cell bodies, suggesting the intriguing possibility of growth cone-specific translational mechanisms for selective mRNA expression.

### Example 9

### Selection of Growth Cone Ribosome Proteins for Epitope Tagging

After identifying that the composition of ribosome proteins in growth cones is distinct from the composition in cell bodies, the present inventors explored the utilization of ribosome affinity purification to identify actively translated mRNA transcripts in growth cones. It has previously been reported that eGFP-tagged RpL10a fusion proteins traffic appropriately to cellular compartments, are successfully incorporated into polysomes for mRNA translation, and enable cell type-specific molecular profiling of translated mRNA transcripts (Heiman et al., 2008; Heiman et al., 2014; Liu et al., 2014). Since RpL10a is not present in growth cones (see FIG. 9), the present inventors sought to develop an analogous strategy to tag a ribosome protein localized to growth cones for ribosome affinity purification.

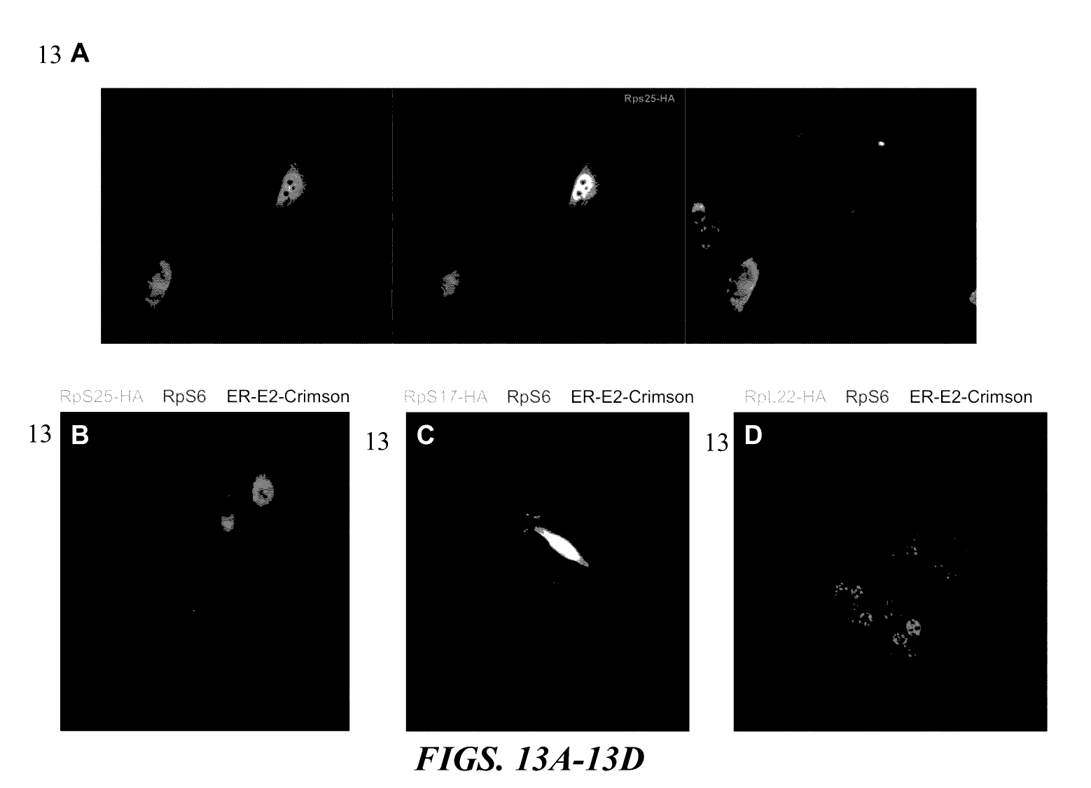

Based on the expression profile of ribosome proteins in CPN growth cones (see FIG. 12), the present inventors selected RpS17, RpS25, and RpL22 to examine as recombinant C-terminally HA-tagged proteins. The present inventors first tested HA-tagged constructs in NIH/3T3 mouse fibroblasts to investigate their subcellular localization as an indicator of their incorporation into mature ribosomes. The present inventors found that HA-tagged RpS25 proteins display an anticipated granular cytosolic distribution, and in many cases co-localize with an ERmarker, E2-Crimson (FIGS. 13A and 13B), suggesting both proper subcellular localization and incorporation into translating ER-associated ribosomes. In contrast, the present inventors found that HA-tagged RpS17 and RpL22 proteins strongly accumulate in the nucleus, and often appear sequestered within nucleoli (FIGS. 13C and 13D). Based on these findings, the present inventors selected HA-tagged RpS25 for investigation of local translation in growth cones in vivo.

It is interesting to note that these results are consistent with a previously reported ribosome protein siRNA knockdown analysis in HeLa cells, which found that siRNA knockdown of RpS25 has little effect on the assembly and function of ribosomes, while RpS17 knockdown perturbs assembly and function of ribosomes (Robledo et al., 2008). While conditional HA-tagged RpL22 mice were generated by another group for cell type-specific ribosome affinity purification (Sanz et al., 2009), it is important to note that the subcellular distribution of HA-tagged RpL22 was not reported.

### Example 10

### Ribosome Affinity Purification from Growth Cones In Vivo

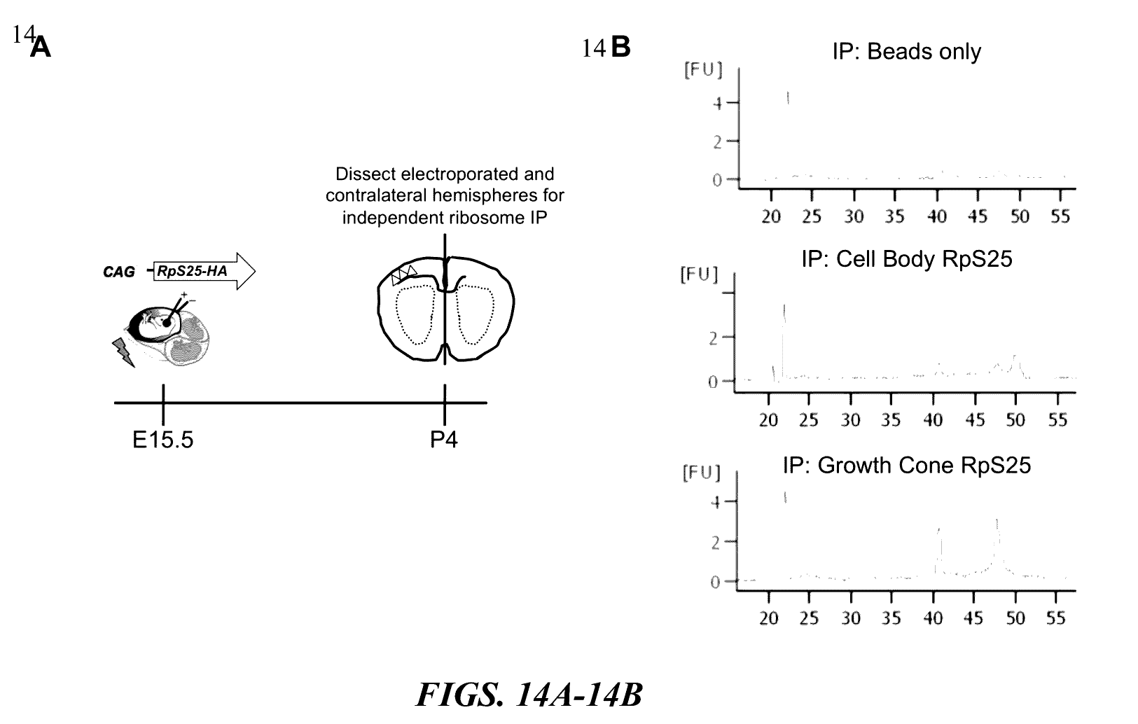

To identify locally translated mRNA transcripts in CPN growth cones in vivo, the present inventors used in utero electroporation to introduce a CAG>RpS25-HA expression plasmid into CPN progenitors at E15.5. At P4, the present inventors selectively dissected the ipsilateral electroporated hemisphere and the contralateral hemisphere independently for ribosome affinity purification (FIG. 14A). As an immunoprecipitation (IP) control sample, the present inventors also performed ribosome affinity purification on non-neocortical brain regions from the same brains. After IP, the present inventors characterized the distribution of purified RpS25-HAassociated RNA using Bioanalyzer analysis. The present inventors verified that RNA was not purified in the control sample (FIG. 14B), and find that this strategy enables purification of translated RNA transcripts selectively from electroporated cell bodies and their growth cones (FIG. 14B). Since evidence for local translation has previously only been reported in vitro, this finding is the first direct in vivo evidence to our knowledge for local translation in growth cones.

Since our strategy enables dual purification of translated CPN cell body and growth cone RNA, ongoing comparative analyses will likely reveal the proportion of transcripts selectively translated in growth cones. RNA-seq analyses are being performed to identify translated mRNA transcripts in growth cones. In addition, bioinformatic analyses are being performed to investigate whether motifs or structural elements in the 3′ untranslated regions of growth cone-localized mRNA transcripts might confer additional translational control.

### DISCUSSION

Enormous complexity among cell types and their connectivity is fundamental to the brain's ability to collectively extract information from the outside world, perform specific computations, and direct behavioral output. To dissect the complexity and diversity among cell types, substantial progress has been made in elucidating transcriptional networks that control the generation and specification of distinct neuron subtypes. Many of these approaches have combined genetic or hodological labeling with fluorescence-activated cell sorting (FACS) to purify specific neuron subtypes, followed by downstream comparative transcriptomic profiling by microarrays or RNAseq (Arlotta et al., 2005; Ayoub et al., 2011; Fishell and Heintz, 2013; Lobo et al., 2006; Molyneaux et al., 2009; Molyneaux et al., 2015). However, such approaches investigating transcriptional dynamics at the cell body level do not yield much insight into how circuits are wired at the subcellular level by growth cones during development. Here, the present inventors describe a generalizable approach to purify growth cones from any desired neuron subtype in the brain, and elucidate the growth cone molecular networks that control the wiring of neural circuits of interest.

The present inventors demonstrate that several labeling approaches can be used to isolate growth cones from desired neuron subtypes. For example, to genetically label a neuron subtype of interest, a fluorescent reporter mouse line can be crossed with a Cre recombinase-expressing mouse line. To label subpopulations of desired neuron subtypes, in utero electroporation can be utilized to spatiotemporally deliver expression plasmids into specific progenitor populations. Furthermore, this approach can easily be combined with genetic mutants or pharmacological treatments that perturb normal connectivity of the brain, enabling potential identification of growth cone-localized molecular dysfunction that might underlie aberrant connectivity phenotypes.

By purifying specific growth cone populations and performing comparative proteomic analyses, the present inventors identify (1) growth cone machinery that appears common to many growth cone populations and (2) proteins that are circuit-specific, including transmembrane adhesion molecules and receptors. The present inventors then devised a subcellular profiling approach by purifying both CPN growth cones and their parent cell bodies. Through comparative RNA-seq, the subcellular distribution of RNA transcripts in CPN was uncovered, finding a specific subset of transcripts substantially enriched in growth cones. Notably, this enriched population includes many noncoding RNA transcripts. As increased evidence suggests that noncoding RNA plays essential roles in the specification of neural identity (Mercer et al, 2008; Molyneaux et al, 2015; Sauvageau et al, 2013), noncoding RNA might also have important functions in the wiring of specific circuits. Taken together, this subcellular profiling approach provides a platform to elucidate the molecular networks involved in wiring specific neural circuits during development.

Notably, this growth cone profiling approach enables investigation of other molecular species that likely have critical functions in the formation of specific neural circuits. For example, as developing axons extend toward their final targets, lipids are critical to plasma membrane expansion as axons increase in surface area up to 20% per day (Pfenninger, 2009). It has also been reported that membrane lipid composition changes as growth cones transform into presynaptic terminals (Martin and Bazan, 1992), suggesting differential functional roles for lipids in axon guidance and synaptogenesis. However, despite the critical importance that lipids have in brain function, the composition of the brain lipidome remains largely unexplored. Interestingly, a recent large-scale mass spectrometry-based comparison of lipid composition across three distinct brain regions and other tissues found that approximately 75% of lipid compounds have distinct expression profiles in brain (Bozek et al., 2015). Given the significant membranous component of axons, it is likely that lipidomic analysis of specific growth cone populations will also yield novel insights into mechanisms that wire neural circuits. Importantly, lipidomics is compatible with proteomics, so it can be readily combined with my subcellular profiling approach in future investigations.

In addition to lipids, while the present inventors excluded the characterization of growth cone microRNA (miRNA) here, they are intriguing regulators of protein translation in growth cones. miRNA have unusually long half-lives of approximately 120 hours (10-fold that of average mRNA transcripts) (Gantier et al., 2011), and recent in vitro evidence has shown that individual miRNAs can regulate local translation of specific mRNA transcripts in axons (Baudet et al., 2012; Hancock et al., 2014). Using this growth cone profiling approach, future deep profiling of miRNAs localized to growth cones, and identification of their target mRNA transcripts, will likely reveal that miRNA play critical roles in shaping the regulatory network of the growth cone transcriptome, perhaps by functioning through competing endogenous RNA (ceRNA) networks (Poliseno et al., 2010; Salmena et al., 2011).

Deep RNA sequencing and computational analyses have recently revealed that another class of RNA termed circular RNA (circRNA), in which the 5′ and 3′ ends covalently link together, is highly enriched in the brain (Rajewsky et al., 2013; You et al, 2015). Intriguingly, it was reported that many circRNAs are localized to dendrites, and are dynamically regulated during periods of synaptogenesis (You et al., 2015), suggesting specific regulatory functions. Because of their increased stability compared to mRNA transcripts (Rajewsky et al., 2013), circRNAs might play additional important regulatory roles in growth cones.

Taken together, the framework provided by this work to profile macromolecules in specific growth cone populations might foster novel insight into how specific neural circuits are wired, and lead to the identification of dysfunctional molecular networks that result in wiring abnormalities. Using such insight, we might achieve understanding of the molecular bases of particular mental illness and neurological disorders, and identify novel drug targets for treating these disorders. Furthermore, this subcellular growth cone profiling approach could potentially be used as a robust biomarker system to validate the efficacy of drugs developed to target molecules implicated in wiring abnormalities.

In Examples 7-10, the present inventors report that growth cone ribosomes are distinct in their composition of ribosome proteins compared to ribosomes in their parent cell bodies. By profiling growth cone and cell body proteomes from a specific neuron subtype, the present inventors identify that growth cone ribosomes specifically lack many 60S subunit proteins that are present in cell body ribosomes. By tagging a ribosome protein present in growth cone ribosomes, the present inventors provide the first in vivo evidence of local translation in growth cones. Given the large distances that growth cones are typically located from their parent cell bodies, local translation is a mechanism that enables growth cones to dynamically regulate their interpretation of molecular guidance cues as they implement circuit connectivity.

Evidence for local translation in axonal compartments has been controversial. While the first evidence for translation in growth cones was reported almost fifty years ago using radiolabeled amino acids (Guiditta et al., 1968; Koenig, 1967), subsequent ultrastructural analyses using electron microscopy led to conflicting views on whether ribosomes were present in axons and growth cones. Several groups reported the presence of ribosomes in the peripheral nervous system in developing rabbit DRG axons (Tennyson, 1970), developing chicken DRG axons (Yamada et al., 1971), developing rat superior cervical ganglion growth cones (Bunge et al., 1973), and adult rat DRG axons at the nodes of Ranvier (Zelena, 1970). In contrast, other groups found no evidence of ribosomes in the central nervous system using electron microscopy, including developing cerebellum axons (del Cerro and Snider, 1967) and in cortical growth cone particles (Pfenninger et al., 1983).

Utilizing biochemical and immunocytochemical approaches, evidence for ribosomes was subsequently reported in squid axons (Giuditta et al., 1991), Mauthner cell axons (Edstrom and Sjostrand, 1969; Koenig and Martin, 1996), mouse cortex growth cones (Bassell et al., 1998), rat sciatic nerve axons (Kun et al., 2007), and rabbit lumbar spinal axons (Koenig et al., 2000). In addition, it was reported that the Netrin-1 receptor DCC directly interacts with elements of the translational machinery, including eukaryotic initiation factors, specific ribosomal 40S and 60S subunit proteins, and assembled monosomes, but not with polysomes (Tcherkezian et al., 2010).

Interestingly, however, the proteomic composition of growth cone ribosomes has not previously been reported. Several recent reports have identified that the presence or absence of specific ribosome proteins can endow ribosomes with translational control specificity. It was recently reported that absence of RpL38 affects patterning of the vertebrate skeleton by affecting the ability to translate particular mRNA transcripts of the Hox family of transcription factors (Kondrashov et al., 2011). During termination of the inflammatory response by macrophages, RpL13a is phosphorylated to stimulate its release from the 60S subunit, which then represses the translation of specific mRNA transcripts that are initially required for the inflammatory response, but are toxic to tissues if continually present at high levels (Mazumder et al., 2003). These examples of specific ribosome proteins conferring translation selectivity raise the possibility that the distinct ribosome protein composition in growth cones regulates specificity of local translation. Intriguingly, transcriptomic profiling of RNA isolated from growth cones in vitro has revealed that mRNA transcripts encoding ribosomal proteins are highly expressed in axons (Andreassi et al., 2010; Gumy et al., 2011; Taylor et al., 2009; Zivraj et al., 2010). This raises the intriguing possibility that, as growth cones progress past intermediate targets, translation of specific ribosome proteins might endow growth cones with regulatory control over protein expression through their ribosomes. It is also intriguing to speculate that several ribosome types might be present in growth cones (and cell bodies).

### Materials and Methods

Mice

Pregnant CD1 mice were obtained from Charles River Laboratories NTSR1::Cre mice were obtained from MMRRC. Ai9 (Rosa26^{flaxSTOPTdTomato}) and ubiquitous expressing CAG-EGFP mice were obtained from Jackson Laboratories. Ubiquitous expressing TdTomato mice were generated by crossing Ddx4::Cre (a germ line delete Cre line, obtained from Jackson Laboratories) with the Ai9 line. The day of vaginal plug detection was designated as embryonic day 0.5 (E0.5), and the day of birth was designated as postnatal day 0 (P0). All mouse studies were approved by the Harvard University IACUC, and were performed in accordance with institutional and federal guidelines.

Microtransplantation

Neurons from GFP+ or TdTomato+ mouse pups were dissociated using partial papain digestion from P0 medial or lateral cortex explants, respectively, and injected at a depth of 100 μm into the medial cortex of dark (GFP−, TdTomato−) littermate hosts at P0. Animals were sacrificed at P7, brains were perfused, fixed, and sectioned at 80 μm on a vibratome (Leica). Tissue sections were imaged using a Nikon 90i fluorescence microscope equipped with an Andor camera. Quantification was performed on full series of non-adjacent sections spanning the length of the transplantation focus in 6 brains from 3 independent experiments.

Plasmids and in Utero Electroporation

Expression plasmids under control of a constitutively active CMV/β-actin promoter were mixed at 2 μg/μL with 0.005% Fast Green, injected in utero into the lateral ventricle of CD1 embryos at E14.5 or E15.5, and electroporated into the neocortical ventricular zone essentially according to (Saito and Nakatsuji, 2001) and (Molyneaux et al., 2005). pCS2>TAG (Trichas et al., 2008) was a gift from S. Srinivas (Addgene plasmid #26772), and pCAG>GFP-GPI (Rhee et al., 2008) was a gift from A. K. Hadjantonakis. pCAG>myr-TdTomato was generated by excision of 2A-H2B-EGFP from pCS2-TAG. For in utero electroporation, expression plasmids were mixed at 2 μg/μL with 0.005% Fast Green, injected in utero into the lateral ventricle of CD1 embryos at E14.5 or E15.5, and electroporated into the neocortical ventricular zone essentially according to (Saito and Nakatsuji, 2001) and (Molyneaux et al., 2005).

Subcellular Fractionation

Postnatal day 2-4 (P2-P4) mice were sacrificed by decapitation, and the forebrain was quickly manually dissected on ice. Brain tissue was immediately homogenized using a glass-Teflon dounce homogenizer with 12 strokes at 900 rpm in ice-cold 320 mM sucrose supplemented with 4 mM HEPES, RNase inhibitors (Promega), and protease/phosphatase inhibitors without EDTA (Halt, Life Technologies). Homogenate was subjected to low speed centrifugation (1,660×g, 15 minutes, 4° C.), and a sample of supernatant was collected as the postnuclear homogenate (PNH). The remaining PNH was layered onto a discontinuous sucrose gradient (830 mM sucrose and 2.66M sucrose cushion), and spun at 242,000×g at 4° C. for 47 min in a vertical rotor (VTi50) using an Optima XPN 80 ultracentrifuge (Beckman Coulter). The growth cone fraction was collected at the interface of 320 mM and 830 mM sucrose solutions. Additional fractions were collected to assay subcellular purity.

Western Blot

Protein samples were quantified using the Qubit protein assay (Invitrogen). 20-30 μg of protein from each sample was boiled for 5 min at 95° C. in Laemmli lysis buffer (2% SDS, 10% glycerol, 0.004% bromophenol blue, 0.125 M Tris HCl, and 5% β-mercaptoethanol), resolved by SDS-PAGE, and transferred to a nitrocellulose or PDVF membrane (Biorad). Membranes were blocked in TBS-T with 5% milk or 5% BSA, and probed with indicated primary antibodies in block solution at 4° C. overnight. Secondary antibodies conjugated with horseradish peroxidase or Alexa 647 were then added for 1 hour at room temperature. Fluorescence or chemiluminescence imaging was performed using a FluorChem M imaging system (Protein Simple). For chemiluminescence, membranes were incubated with enhanced chemiluminescent substrate (Thermo Scientific) prior to imaging.

Trypsin Protection Assays

Isolated growth cone fractions were split into four samples and treated with distinct combinations of Triton X-100 (0.3%) and 0.25% Trypsin or 10 units of recombinant RNase One (Promega) for 1 hour with constant rotation. RNA and protein were subsequently extracted from samples using the Allprep RNA/protein kit according to manufacturer's instructions (Qiagen).

Antibodies

The following antibodies are commercially available and used according to manufacturer's suggestions for Western blotting: mouse α-GAP43 (1:500, EMD Millipore # MAB347); mouse α-MAP2 (1:500, Sigma # M1406); rabbit α-Tujl (1:1000, Cell Signaling #5666); rabbit a-Histone H3 (1:1000, Cell Signaling #4499); rabbit α-ERp72 (1:1000, Cell Signaling #5033); rabbit α-HSP-90 (1:1000, #4877); rabbit α-eIF4E (1:1000, Cell Signaling #9742); rabbit α-S3 (1:1000, Cell Signaling #9538); rabbit α-S6 (1:1000, Cell Signaling #2317); rabbit α-RpL7a (1:1000, Cell Signaling #2415); rabbit α-RpL26 (1:1000, Cell Signaling #5400); rabbit α-RpL28 (1:500, Santa Cruz #Sc-50362); mouse α-RpL10a (1:500, Abnova # H00004736-M01). Secondary antibodies were: goat α-rabbit HRP (1:2500, Cell Signaling #7074); goat α-mouse HRP (1:2500, Thermo Scientific #32430); or goat α-mouse 647 (1:2500, Life Technologies # A-21236).

Fluorescent Small Particle Sorting

Growth cones were sorted on a BD Special Order FACS Aria III customized for small particle sorting. Prior to sorting, the instrument was decontaminated with bleach and RNase-free water to remove RNases. The instrument was then calibrated with fluorescent beads in the sub-micron range. Purified growth cones were diluted 1:2 in 1×PBS (RNase-free), and scatter gating was adjusted for single growth cone particles, which ranged from 0.3 to 0.8 μm in diameter. Fluorescent populations of growth cones were sorted directly in RLT buffer (Qiagen) to dissociate membranes and stabilize RNA/protein complexes for subsequent RNA and protein extraction.

RNA and Protein Isolation from Growth Cones

RNA and protein were isolated in parallel from the same samples using the Allprep DNA/RNA/protein kit according to manufacturer's instructions (Qiagen), except protein was concentrated and purified using Acetonc/TCA precipitation prior to proteomics. DNase treatment was included to digest genomic DNA. RNA quality was assessed using an Agilent 2100 Bioanalyzer, and RNA integrity numbers (RINs) for all samples were between 8.0 and 9.5.

Proteomics

Purified growth cone and cell body proteins were subjected to on-pellet trypsin/Lys-C digestion. Samples were injected through a Waters NanoACQUITY UPLC directly coupled to electrospray ionization feeding a linear ion trap quadropole (LTQ Velos) coupled to an Orbitrap Elite mass analyzer. Clustering analysis was performed using the STRING database mined for documented protein interactions.

RNA-Sequencing

Equal masses of high quality RNA (RIN>8) from sorted cell bodies and growth cones were made into cDNA libraries using the Ovation Single Cell RNA-seq system, which uses random hexamer primers that enrich for non-rRNA sequences (NuGEN). Cell body and growth cone cDNA libraries were uniquely barcoded and sequenced together on the Illumina Hiseq 2500, generating 100-bp paired-end reads. For bioinformatic processing, we used FASTX-Toolkit, Tophat2, and Bowtie2 with default parameters to preprocess, align, and assemble reads into transcripts and estimate abundance (Langmead et al., 2009; Trapnell et al., 2012). Aligned reads and the UCSC reference transcriptome .gtf file (downloaded from the UCSC genome browser) were used as input into Cufflinks2 using default parameters for expression quantification, and then into Cuffdiff2 (Trapnell et al., 2012) for differential testing between growth cone and cell body RNA expression.

Ribosome Affinity Purification

Electroporated pups were sacrificed by decapitation at P4, and cortical hemispheres were dissected and separated. Electroporated cortices were pooled into “soma” sample, contralateral cortices were pooled together into “growth cone” sample, and dissected tissues (including cerebellum and midbrain) were pooled together into “control” sample. Tissue from each sample was homogenized using a glass-Teflon dounce homogenizer with 12 strokes at 900 rpm in 5 mL ice-cold homogenization buffer (150 mM KCl, 10 mM MgCl2, 20 mM HEPES, 0.5 mM DTT, 100 μg/mL cycloheximide, 10 μg/mL RNasin, and protease/phosphatase inhibitors without EDTA (Halt, Life Technologies), pH 7-7.5).

Homogenate was subjected to low speed centrifugation (1,660×g, 10 minutes, 4° C.) to pellet nuclei and cellular debris, and a sample of the supernatant was collected as the postnuclear fraction. To the remaining postnuclear fraction, NP-40 detergent (nonylphenyl polyethylene glycol) was added to a final concentration of 1%, and DHPC phospholipid (1,2-diheptanoyi-sn-glycero-3-phosphocholine; Avanti Polar Lipids) was added to a final concentration of 30 mM. After extraction on ice for 5 minutes, the postmitochondrial fraction was generated by centrifugation (15,000×g, 15 minutes, 4° C.), and a sample was collected.

To the remaining postmitochondrial fraction, ascites fluid containing 20-30 μg IgG1 of MαHA.11 (16B12, Covance MMS-101R) was added to each sample, and incubated for 4 hours at 4° C. with constant rotation. 200 μL of lysis-buffer-washed Protein G-coated Dynabeads (Life Technologies) were added to each sample, and incubated for 14 hours at 4° C. with constant rotation. Beads were washed four times with full resuspension in high salt buffer (350 mM KCl, 10 mM MgCl2, 20 mM HEPES, 0.5 mM DTT, 100 μg/mL cycloheximide, 1% NP-40, 30 mM DHPC, pH 7-7.5). Finally, ribosome-associated RNA and proteins were eluted from the beads using RLT buffer (Qiagen, without β-mercaptoethanol), and RNA/protein were isolated using the Allprep DNA/RNA/protein kit (Qiagen) per manufacturer's instructions. RNA distribution and quality were assessed using an Agilent 2100 Bioanalyzer.

### REFERENCES

Abu-Elneel, K., Liu, T., Gazzaniga, F. S., Nishimura, Y., Wall, D. P., Geschwind, D. H., Lao, K., and Kosik, K. S. (2008). Heterogeneous dysregulation of microRNAs across the autism spectrum. Neurogenetics 9, 153-161.
Alcamo, E. A., Chirivella, L., Dautzenberg, M., Dobreva, G., Farinas, I., Grosschedl, R., and McConnell, S. K. (2008) Satb2 regulates callosal projection neuron identity in the developing cerebral cortex. Neuron 57, 364-377.
Andreassi, C., Zimmermann, C., Mitter, R., Fusco, S., De Vita, S., Saiardi, A., and Riccio, A. (2010). An NGF-responsive element targets myoinositol monophosphatase-1 mRNA to sympathetic neuron axons. Nat Neurosci 13, 291-301.
Antar, L. N., Li, C., Zhang, H., Carroll, R. C., and Bassell, G. J. (2006). Local functions for FMRP in axon growth cone motility and activity-dependent regulation of filopodia and spine synapses. Mol Cell Neurosci 32, 37-48.
Arana, G. W. (2000). An overview of side effects caused by typical antipsychotics. J Clin Psychiatry 61, Suppl 5-11.
Arlotta, P., Molyneaux, B. J., Chen, J., Inoue, J., Kominami, R., and Macklis, J. D. (2005). Neuronal subtype-specific genes that control corticospinal motor neuron development in vivo. Neuron 45, 207-221.
Avesar, D., and Gulledge, A. T. (2012). Selective serotonergic excitation of callosal projection neurons. Front Neural Circuits 6, 1-11.
Ayoub, A. E., Oh, S., Xie, Y., Leng, J., Cotney, J., Dominguez, M. H., Noonan, J. P., and Rakic, P. (2011). Transcriptional programs in transient embryonic zones of the cerebral cortex defined by high-resolution mRNA sequencing. Proc Natl Acad Sci USA 108, 14950-14955.
Azim, E., Jabaudon, D., Fame, R. M., and Macklis, J. D. (2009). Sox6 controls dorsal progenitor identity and interneuron diversity during neocortical development. Nat Neurosci 12, 1238-1247.
Azim, E., Shnider, S. J., Cederquist, G. Y., Sohur, U. S., and Macklis, J. D. (2009). Lmo4 and Climl progressively delineate cortical projection neuron subtypes during development. Cereb Cortex 19 (Suppl 1), i62-i69.
Barnes, A. P., Lilley, B. N., Pan, Y. A., Plummer, L. J., Powell, A. W., Raines, A. N., Sanes, J. R., and Polleux, F. (2007). LKB1 and SAD kinases define a pathway required for the polarization of cortical neurons. Cell 129, 549-563.
Barnes, A. P., and Polleux, F. (2009). Establishment of axon-dendrite polarity in developing neurons. Annu Rev Neurosci 32, 347-381.
Bartel, D. P. (2004). MicroRNAs: genomics, biogenesis, mechanism, and function. Cell 125 116, 281-297.
Barttfeld, P., Wicker, B., Cukier, S., Navarta, S., Lew, S., and Sigman, M. (2011). A bigworld network in ASD: dynamical connectivity analysis reflects a deficit in long-range connections and an excess of short-range connections. Neuropsychologia 49, 254-263.)
Bassell, G. J., Zhang, H., Byrd, A. L., Femino, A. M., Singer, R. H., Taneja, K. L., Lifshitz, L. M., Herman, I. M., and Kosik, K. S. (1998). Sorting of β-actin mRNA and protein to neurites and growth cones in culture. J Neurosci 18, 251-265.
Baudet, M. L., Zivraj, K. H., Abreu-Goodger, C., Muldal, A., Armisen, J., Blenkiron, C., Goldstein, L. D., Miska, E. A., and Holt, C. E. (2012). miR-124 acts through CoREST to control onset of Sema3A sensitivity in navigating retinal growth cones. Nat Neurosci 15, 29-38.
Bedogni, F., Hodge, R. D., Elsen, G. E., Nelson, B. R., Daza, R. A., Beyer, R. P., Bammler, T. K., Rubenstein, J. L., and Hevner, R. F. (2010). Tbrl regulates regional and laminar identity of postmitotic neurons in developing neocortex. Proc Natl Acad Sci USA 107, 13129-13134.
Ben-Sham, A., Garreua de Loubresse, N., Melnikov, S., Jenner, L., Yusupova, G., and Yusupov, M. (2011). The structure of the eukaryotic ribosome at 3.0 Å resolution. Science 1524-1529.
Bentley, D., and Caudy, M. (1983). Pioneer axons lose directed growth after selective killing of guidepost cells. Nature 304, 62-65.
Bentley, D., and Keshishian, H. (1982). Pathfinding by peripheral pioneer neurons in grasshoppers. Science 218, 1082-1099.
Besse, F., and Ephrussi, A. (2008). Translational control of localized mRNAs: restricting protein synthesis in space and time. Nat Rev Mol Cell Biol 9, 971-980,
Biesemann, C., Grønborg, M., Luquet, E., Wichert, S. P., Bernard, V., Bungers, S. R., Cooper, B., Varoqueaux, F., Li, L., et al. (2014). Proteomic screening of glutamatergic mouse brain synaptosomes isolated by fluorescence activated sorting. EMBO Journal 33, 157-170.
Bloom, D. E., Cafiero, E. T., Jane-Llopis, E., Abrahams-Gessel, S., Bloom, L. R., Fathima, S., Feigl, A. B., Gaziano, T., Mowafi, M., Pandya, A., et al. (2011). The Global Economic Burden of Noncommunicable Diseases. Geneva: World Economic Forum. Boersema, P. J., Raijmakers, R., Lemeer, S., Mohammed, S., and Heck, A. J. R. (2009). Multiple peptide stable isotope dimethyl labeling for quantitative proteomics. Nat Protoc 4, 484-494.
Borlongan, C. V., Burns, J., Tajiri, N., Stahl, C. E., Weinbren, N. L., Shojo, H., Sanberg, P. R., Emerich, D. F., Kaneko, Y., and van Loveren, H. R. (2013). Epidemiological survey-based formulae to approximate incidence and prevalence of neurological disorders in the United States: a meta-analysis. PLoS One 8, e78490.
Boyden, E. S., Zhang, F., Bamberg, E., Nagel, G., and Deisseroth, K. (2005). Millisecond-timescale, genetically targeted optical control of neural activity. Nat Neurosci 8, 1263-1268.
Bozek, K., Wei, Y., Yan, Z., Liu, X., Xiong, J., Sugimoto, M., Tomita, M., Paabo, S., Sherwood, C. C., Hof, P. R., et al. (2015). Organization and evolution of brain lipidome revealed by large-scale analysis of human, chimpanzee, macaque, and mouse tissues. Neuron 85, 695-702.
Brady, S. T. (1991). Molecular motors in the nervous system. Neuron 7, 521-533.
Bray, D. (1970). Surface movements during the growth of single explanted neurons. Proc Natl Acad Sci USA 65, 905-910.
Briscoe, J., Pierani, A., Jessell, T. M., and Ericson, J. (2000). A homeodomain protein code specifies progenitor cell identity and neuronal fate in the ventral neural tube. Cell 101, 435-445.
Briscoe, J., Sussel, L., Serup, P., Hartigan-O'Connor, D., Jessell, T. M., Rubenstein, J. L. R., and Ericson, J. (1999). Homeobox gene Nkx2.2 and specification of neuronal identity by graded Sonic hedgehog signaling. Nature 398, 622-627.
Britanova, O., de Juan Romero, C., Cheung, A., Kwan, K. Y., Schwark, M., Gyorgy, A., Vogel, T., Akopov, S., Mitkovski, M., Agoston, D., et al. (2008) Satb2 is a postmitotic determinant for upper-layer neuron specification in the neocortex. Neuron 57, 378-392.
Brittis, P. A., Lu, Q., and Flanagan, J. G. (2002). Axonal protein synthesis provides a mechanism for localized regulation at an intermediate target. Cell 110, 223-235.
Bunge, M. B. (1973). Fine structure of nerve fibers and growth cones of isolated sympathetic neurons in culture. J Cell Biol 56, 713-735.
Burry, R. W. (1991). Transitional elements with characteristics of both growth cones and presynaptic terminals observed in cell cultures of cerebellar neurons. J Neurocytol 20, 124-132.
Cajal, S. R. (1890). A quelle époque apparaissent les expansions des cellules nerveuses de la moëlle épinière du poulet? Anat Anz 5, 609-613, 621-631.
Cajal, S. R. (1892). La rétine des vertébrés. La Cellule 9, 121-133.
Cajal, S. R. (1909) Histologic du Système Nerveux de l'Homme et des Vertébrés. Translated by Leon Azoulay. Masson: Paris.
Campbell, D. S., and Holt, C. E. (2001). Chemotropic responses of retinal growth cones mediated by rapid local protein synthesis and degradation. Neuron 32, 1013-1026.
Caudy, M., and Bentley, D. (1986). Pioneer growth cone steering along a series of neuronal and non-neuronal cues of different affinities. J Neurosci 6, 1781-1795.
del Cerro, M. P., and Snyder, R. S. (1968). Studies on the developing cerebellum. Ultrastructure of the growth cones. J Comp Neurol 133, 341-362.
Chao, D. L., Ma, L., and Shen, K. (2009). Transient cell-cell interactions in neural circuit formation. Nat Rev Neurosci 10, 262-271.
Charron, F., Stein, E., Jeong, J., McMahon, A. P., and Tessier-Lavigne, M. (2003). The morphogen sonic hedgehog is an axonal chemoattractant that collaborates with netrin-1 in midline axon guidance. Cell 113, 11-23.
Chauvet, S., Cohen, S., Yoshida, Y., Fekrane, L., Livet, J., Gayet, O., Segu, L., Buhot, M. C., Jessell, T. M., Henderson, C. E., et al. (2007). Gating of Sema3E/PlexinD1 signaling by Neuropilin-1 switches axonal repulsion to attraction during brain development. Neuron 56, 807-822.
Chen, B., Schaevitz, L. R., and McConnell, S. K. (2005). Fezl regulates the differentiation and axon targeting of layer 5 subcortical projection neurons in cerebral cortex. Proc Natl Acad Sci USA 102, 17184-17189.
Chen, Y., Zhu, J., Lum, P. Y., Yang, X., Pinto, S., MacNeil, D. J, Zhang, C., Lamb, J., Edwards, S., Sieberts, S. K., et al. (2008). Variations in DNA elucidate molecular networks that cause disease. Nature 452, 429-435.
Chen, Z., Gore, B. B., Long, H., Ma, L., and Tessier-Lavigne, M. (2008). Alternative splicing of the Robo3 axon guidance receptor governs the midline switch from attraction to repulsion. Neuron 58, 325-332.
Cheng, H. J., and Flanagan, J. G. (1994). Identification and cloning of ELF-1, a developmentally expressed ligand for the Mek4 and Sek receptor tyrosine kinases. Cell 79, 157-168.
Cheng, H. J., Nakamoto, M., Bergemann, A. D., and Flanagan, J. G. (1995). Complementary gradients in expression and binding of ELF-1 and Mek4 in development of the topographic retinotectal projection map. Cell 82, 371-381.
Colak, D., Ji, S. J., Porse, B. T., and Jaffrey, S. R. (2013). Regulation of axon guidance by compartmentalized nonsense-mediated mRNA decay. Cell 153, 1252-1265.
Cox, L. J., Hengst, U., Gurskaya, N. G., Lukyanov, K. A., and Jaffrey, S. R. (2008). Intraaxonal translation and retrograde trafficking of CREB promotes neuronal survival. Nat Cell Biol 10, 149-159.
Dalla Torre di Sanguinetto, S., Dasen, J. S., and Arber, S. (2008). Transcriptional mechanisms controlling motor neuron diversity and connectivity. Curr Opin Neurobiol 18, 36-43.
Darnell, J. C., Jensen, K. B., Jin, P., Brown, V., Warren, S. T., and Darnell, R. B. (2001). Fragile X mental retardation protein targets G quartet mRNAs important for neuronal function. Cell 107, 489-499.
Darnell, J. C., Van Driesche, S. J., Zhang, C., Hung, K. Y., Mele, A., Fraser, C. E., Stone, E. F., Chen, C., Fak, J. J., Chi, S. W., et al. (2011). FMRP stalls ribosomal translocation on mRNAs linked to synaptic function and autism. Cell 146, 247-261.
Dasen, J. S., de Camilli, A., Wang, B., Tucker, P. W., and Jessell, T. M. (2008). Hox repertoires for motor neuron diversity and connectivity gated by a single accessory factor, FoxP1. Cell 134, 304-316.
Dasen, J. S., Liu, J. P., and Jessell, T. M. (2003). Motor neuron columnar fate imposed by sequential phases of Hox-c activity. Nature 425, 926-933.
Dasen, J. S., Tice, B. C., Brenner-Morton, S., and Jessell, T. M. (2005). A Hox regulatory network establishes MN pool identity and target-muscle connectivity. Cell 123, 477-491.
Demireva, E. Y., Shapiro, L. S., Jessell, T. M., and Zampieri, N. (2011). Motor neuron position and topographic order imposed by β- and γ-Catenin activities. Cell 147, 641-652.
Dent, E. W., and Gertler, F. B. (2003). Cytoskeletal dynamics and transport in growth cone motility and axon guidance. Neuron 40, 209-227.
Dickson, B. J., and Zou, Y. (2010). Navigating intermediate targets: the nervous system midline. Cold Spring Harb Perspect Biol 2, a002055.
Ding, J. B., Oh. W. J., Sabatini, B. L., and Gu, C. (2011). Semaphorin 3E-Plexin-D1 signaling controls pathway-specific synapse formation in the striatum. Nat Neurosci 15, 215-223.
Doyle, J. P., Dougherty, J. D., Heiman, M., Schmidt, E. F., Stevens, T. R., Ma, G., Bupp, S., Shrestha, P., Shah, R. D., Doughty, M. L., et al. (2008). Application of a translational profiling approach for the comparative analysis of CNS cell types. Cell 135, 749-762.
Duncan, J. E., and Goldstein, L. S. B. (2006). The genetics of axonal transport and axonal transport disorders. PLoS Genet 2, e124.
Edstrom, A., and Sjostrand, J. (1969). Protein synthesis in the isolated Mauthner nerve fibre of goldfish. J Neurochem 16, 67-81.
Edwards, S. L., Breesley, J., French, J. D., and Dunning, A. M. (2013). Beyond GWASs: Illuminating the dark road from association to function. Am J Hum Genet 93, 779-797.
Edwards, S. L., Yu, S., Hoover, C. M., Phillips, B. C., Richmond, J. E., and Miller, K. G. (2013). An organelle gatekeeper function for Caenorhabditis elegans UNC-16 (JIP3) at the axon initial segment. Genetics 194, 143-161.
Engle, E. C. (2010). Human genetic disorders of axon guidance. Cold Spring Harb Prespect Biol 2, a001784.
Ericson, E., Gebbia, M., Heisler, L. E., Wildenhain, J., Tyers, M., Giaever, G., and Nislow, C. (2008). Off-target effects of psychoactive drugs revealed by genome-wide assays in yeast. PLoS Genet 4, el000151.
Estrada-Bernel, A., Sanford, S. D., Sosa, L. J., Simon, G. C., Hansen, K. C., and Pfenninger, K. H. (2012). Functional complexity of the axonal growth cone: a proteomic analysis. PLoS One 7, e31858.
Etienne-Manneville, S., and Hall, A. (2002). Rho GTPases in cell biology. Nature 420, 629-635.
Fame, R. M., MacDonald, J. L., and Macklis, J. D. (2011). Development, specification, and diversity of callosal projection neurons. Trends Neurosci 34, 41-50.
Farina, K. L., Huttelmaier, S., Musunuru, K., Darnell, R., and Singer, R. H. (2003). Two ZBP1 KH domains facilitate b-actin mRNA localization, granule formation, and cytoskeletal attachment. J Cell Biol 160, 77-87.
Feldheim, D. A., Kim, Y., Bergemann, A. D., Frisen, J., Barbacid, M., and Flanagan, J. G. (2000). Genetic analysis of ephrin-A2 and ephrin-A5 shows their requirement in multiple aspects of retinocollicular mapping. Neuron 25, 563-574.
Feldheim, D. A., and O'Leary, D. D. M. (2010). Visual map development: Bidirectional signaling, bifunctional guidance molecules, and competition. Cold Spring Harb Perspect Biol 2, a001768.
Filipe, V., Hawe, A. and Jiskoot, W. (2010). Critical evaluation of Nanoparticle Tracking Analysis (NTA) by NanoSight for the measurement of nanoparticles and protein aggregates. Pharm Res 27, 796-810
Finlay, B. L., and Darlington, R. B. (1995) Linked regularities in the development and evolution of mammalian brains. Science 268, 1578-1584.
Fishell, G., and Heintz, N. (2013). The neuron identity problem: form meets function. Neuron 80, 602-612.
Flames, N., Pla, R., Gelman, D. M., Rubenstein, J. L., Puelles, L., and Marin, O. (2007) Delineation of multiple subpallial progenitor domains by the combinatorial expression of transcriptional codes. J Neurosci 27, 9682-9695.
Fode, C., Ma, Q., Casarosa, S., Ang, S. L., Anderson, D. J., and Guillemot, F. (2000). A role for neural determination genes in specifying the dorsoventral identity of telencephalic neurons. Genes Dev 14, 67-80.
Fornito, A., Zalesky, A., and Breakspear, M. (2015). The connectomics of brain disorders. Nat Rev Neurosci 16, 159-172.
Frisen, J., Yates, P. A., McLaughlin, T., Friedman, G. C., O'Leary, D. D. M., and Barbacid, M. (1998). Ephrin-A5 (AL-1/RAGS) is essential for proper retinal axon guidance and topographic mapping in the mammalian visual system. Neuron 20, 235-243.
Fromer, M., Pocklington, A. J., Kavanagh, D. H., Williams, H. J., Dwyer, S., Gormley, P., Georgieva, L., Rees, E., Palta, P., Ruderfer, D. M., et al. (2014). De novo mutations in schizophrenia implicate synaptic networks. Nature 506, 179-184.
Gantier, M. P., McCoy, C. E., Rusinova, I., Saulep, D., Wang, D., Xu, D., Irving, A. T., Behlke, M. A., Hertzog, P. J., MacKay, F., and Williams, B. R. (2011). Analysis of microRNA turnover in mammalian cells following Dicerl ablation. Nucleic Acids Res 39, 5692-5703.
Gardiol, A., Racca, C., and Triller, A. (1999). Dendritic and postsynaptic protein synthetic machinery. J Neurosci 19, 168-179.
Georgi, B., Craig, D., Kember, R. L., Liu, W., Lindquist, I., Nasser, S., Brown, C., Egeland, J. A., Paul, S. M., and Bucan, M. (2014). Genomic view of bipolar disorder revealed by whole genome sequencing in a genetic isolate. PLoS Genet 10, e1004229.
Geschwind, D. H., and Levitt, P. (2007). Autism spectrum disorders: developmental disconnection syndromes. Curr Opin Neurobiol 17, 103-111.
Ghashghaei, H. T., Lai, C., and Anton, E. S. (2007). Neuronal migration in the adult brain: are we there yet? Nat Rev Neurosci 8, 141-151.
Girard, S. L., Gauthier, J., Noreau, A., Xiong, L., Zhou, S., Jouan, L., Dionee-Laporte, A., Spiegelman, D., Henrion, E., Diallo, O., et al. (2011). Increased exonic de novo mutation rate in individuals with schizophrenia. Nature Genet 43, 860-863.
Gitler, A. D., Lu, M. M., and Epstein, J. A. (2004). PlexinD1 and semaphorin signaling are required in endothelial cells for cardiovascular development. Dev Cell 5, 107-116.
Giuditta, A., Dettbarn, W. D., and Brzin, M. (1968). Protein synthesis in the isolated giant axon of the squid. Proc Natl Acad Sci USA 59, 1284-1287.
Giuditta, A., Menichini, E., Capano, C., Langella, M., Martin, R., Castigli, E., and Kaplan, B. B. (1991). Active polysomes in the axoplasm of the squid giant axon. J Neurosci Res 28, 18-28.
Gomez, T. M., and Letourneau, P. C. (2014). Actin dynamics in growth cone motility and navigation. J Neurochem 129, 221-234.
Gong S., Doughty, M., Harbaugh, C. R., Cummins, A., Hatten, M. E., Heintz, N., and Gerfen C. R. (2007). Targeting Cre recombinase to specific neuron populations with bacterial artificial chromosome constructs. J Neurosci 27, 9817-9823.
Govindarajan, A., Israely, I., Huang, S. Y., and Tonegawa, S. (2011). The dendritic branch is the preferred integrative unit for protein synthesis-dependent LTP. Neuron 69, 132-146.
Gray, P. A., Fu, H., Luo, P., Zhao, Q., Yu, J., Ferrari, A., Tenzen, T., Yuk, D. I., Tsung, E. F., Cai, Z., et al. (2004). Mouse brain organization revealed through direct genomescale TF expression analysis. Science 306, 2255-2257.
Grienberger, C., and Konnerth, A. (2012). Imaging calcium in neurons. Neuron 73, 862-885.
Grivas, P. D., Kiaris, H., and Papvassiliou, A. G. (2011). Trackling transcription factors: challenges in therapy. Trends Mol Med 17, 537-538.
Gu, C., Yoshida, Y., Livet, J. Reimert, D. V., Mann, F., Merte, J., Henderson, C. E., Jessell, T. M., Kolodkin, A. L., and Ginty, D. D. (2005). Semaphorin 3E and plexin-D1 control vascular pattern independently of neuropilins. Science 307, 265-268.
Gumy, L. F., Yeo, G. S., Tung, Y. C., Zivraj, K. H., Willis, D., Coppola, G., Lam, B. Y., Twiss, J. L. et al. (2011). Transcriptome analysis of embryonic and adult sensory axons reveals changes in mRNA repertoire localization. RNA 17, 85-98.
Guo, C., Eckler, M. J., McKenna, W. L., McKinsey, G. L., Rubenstein, J. L. R., and Chen, B. (2013). Fezf2 expression identifies a multipotent progenitor for neocortical projection neurons, astrocytes, and oligodendrocytes. Neuron 80, 1167-1174.
Guthrie, P. B., Lee, R. E., and Kater, S. B. (1989). A comparison of neuronal growth cone and cell body membrane: electrophysiological and ultrastructural properties. J Neurosci 9, 3596-3605.
György, B., Szabo, T. G., Pasztoi, M., Pal, Z., Misjak, P., Aradi, B., Laszlo, V., Pallinger, E., Pap, E., Kittel, A., et al. (2011). Membrane vesicles, current state-of-the-art: emerging role of extracellular vesicles. Cell Mol Life Sci 68, 2667-2688.
Hammond, S. M., Boettcher, S., Caudy, A. A., Kobayashi, R., and Hannon, G. J. (2001). Argonaute2, a link between genetic and biochemical analyses of RNAi. Science 293, 1146-1150.
Hancock, M. L., Preitner, N., Quan, J., and Flanagan, J. G. (2014). MicroRNA-132 is enriched in developing axons, locally regulates Rasal mRNA, and promotes axon extension. J Neurosci 34, 66-78.
Harris, J. A., Hirokawa, K. E., Sorensen, S. A., Gu, H., Mills, M., Ng, L. L., Bohn, P., Mortrud, M., et al. (2014). Anatomical characterization of Cre driver mice for neural circuit mapping and manipulation. Front Neural Circuits 8, 1-16.
Harris, W. A., Holt, C. E., and Bonhoeffer, F. (1987). Retinal axons with and without their somata, growing to and arborizing in the tectum of Xenopus embryos: a time-lapse video study of single fibres in vivo. Development 101, 123-133.
Harrison, J. G. (1907). Observations on the living developing nerve fiber. Anat Rec 1, 116-118. Harrison, J. G. (1910). The outgrowth of the nerve fiber as a mode of protoplasmic movement. J Exp Zool 9, 787-848.
Haug, H. (1987) Brain sizes, surfaces, and neuronal sizes of the cortexcerebri: a stereological investigation of man and his variability and a comparison with some mammals (primates, whales, marsupials, insectivores, and one elephant). Am J Anat 180, 126-142.
He, M., Liu, Y., Wang, X., Zhang, M. O., Hannon, G. J., and Huang, Z. J. (2012). Celltype-based analysis of microRNA profiles in the mouse brain. Neuron 73, 35-48.
Heiman, M., Kulicke, R., Fenster, R. J., Greengard, P., and Heintz, N. (2014). Cell type specific mRNA purification by translating ribosome affinity purification (TRAP). Nat Protoc 9, 1282-1291.
Heiman, M., Schaefer, A., Gong, S., Peterson, J. D., Day, M., Ramsey, K. E., Suárez-Fariñas, M., Schwarz, C., Stephan, D. A., et al. (2008). A translational profiling approach for the molecular characterization of CNS cell types. Cell 135, 738-748.
Hengst, U., Cox, L. J., Macosko, E. Z., and Jaffrey, S. R. (2006). Functional and selective RNA interference machinery in axonal growth cones. J Neurosci 26, 5727-5732.
Herculano-Houzel, S. (2009). The human brain in numbers: A linearly scaled-up primate brain. Front Hum Neurosci 3, 31.
Hippenmeyer, S., Kramer, I., and Arber, S. (2004). Control of neuronal phenotype: what targets tell the cell bodies. Trends Neurosci 27, 482-488.
Hirokawa, N., Niwa, S., and Tanaka, Y. (2010). Molecular motors in neurons: transport mechanisms and roles in brain function, development, and disease. Neuron 68, 610-638.
Hodge, L. K., Klassen, M. P., Han, B. X., Yiu, G., Hurrell, J., Howell, A., Rousseau, G., Lemaigre, F., Tessier-Lavigne, M., and Wang, F. (2007). Retrograde BMP signaling regulates trigeminal sensory neuron identities and the formation of precise face maps. Neuron 55, 572-586.
Holt, C. E., and Bullock, S. L. (2009). Subcellular mRNA localization in animal cells and why it matters. Science 326, 1212-1216.
Holt, C. E., and Schuman, E. M. (2013). The central dogma decentralized: new perspectives on RNA function and local translation in neurons. Neuron 80, 648-657.
Hong, K., Nishiyama, M., Henley, J., Tessier-Lavigne, M., and Poo, M. (2000). Calcium signalling in the guidance of nerve growth by netrin-1. Nature 403, 93-98.
Hopker, V. H., Shewan, D., Tessier-Lavigne, M., Poo, M., and Holt, C. (1999). Growthcone attraction to netrin-1 is converted to repulsion by laminin-1. Nature 401, 69-73.
Horton, A. C., and Ehlers, M. D. (2003). Dual modes of endoplasmic reticulum-to-Golgi transport in dendrites revealed by live-cell imaging. J Neurosci 23, 6188-6199.
Howe, C. L., and Mobley, W. C. (2005). Long-distance retrograde neurotrophic signaling. Curr Opin Neurobiol 15, 40-48.
Huang, Z. J., and Zeng, H. (2013). Genetic approaches to neural circuits in the mouse. Annu Rev Neurosci 36, 183-215.
Hughes, A. (1953). The growth of embryonic neurites; a study of cultures of chick neural tissues. J Anat 87, 150-162.
Hussman, J. P., Chung, R. H., Griswold, A. J., Jaworski, J. M., Salyakinda, D., Ma, D., Konidari, I., Whitehead, P. L., Vance, J. M., Martin, E. R., et al. (2011). A noise-reduction GWAS analysis implicates altered regulation of neurite outgrowth and guidance in autism. Mol Autism 2, 1.
Huttelmaier, S., Zenklusen, D., Lederer, M., Dictenberg, J., Lorenz, M., Meng, X., Bassell, G. J., Condeelis, J., and Singer, R. H. (2005). Spatial regulation of β-actin translation by Src-dependent phosphorylation of ZBP1. Nature 438, 512-515.
Hyman, S. E. (2013). Psychiatric drug development: diagnosing a crisis. Cerebrum 2013, 5.
Igarashi, M. (2014). Proteomic identification of the molecular basis of mammalian CNS growth cones. Neurosci Res 88, 1-15.
Imai, T., Suzuki, M., and Sakano, H. (2006). Odorant receptor-derived camp signals direct axonal targeting. Science 314, 657-661.
Imai, T., Yamazaki, T., Kobayakawa, R., Kobayakawa, K., Abe, T., Suzuki, M., and Sakano, H. (2009). Pre-target axon sorting establishes the neural map topography. Science 325, 585-590.
Innocenti, G. M., Ansermet, F., and Parnas, J. (2003). Schizophrenia, neurodevelopment, and corpus callosum. Mol Psychiatry 8, 261-274.
Insel, T. R. (2008). Assessing the economic costs of serious mental illness. Am J Psychiatry 165, 703-711.
Insel, T. R., Landis, S. C., and Collins, F. S. (2013). NIH BRAIN Initiative: Research priorities. Science 340, 687-688.
Insel, T. R., Voon, B., Nye, J. S., Brown, V. J., Altevogt, B. M., Bullmore, E. T., Goodwin, G. M., Howard, R. J., Kupfer, D. J., Marston, H. M., et al. (2013). Innovative solutions to novel drug development in mental health. Neurosci Biobehav Rev 37, 2438-2444.
Iossifov, I., Ronemus, M., Levy, D., Wang, Z., Hakker, I., Rosenbaum, J., Yamrom, B., Lee, Y. H., Narzisi, G., Leotta, A., et al. (2012). De novo gene disruptions in children on the autistic spectrum. Neuron 74, 285-299.
Jen, J. C., Chan, W. M., Bosley, T. M., Wan, J., Can, J. R., Rub, U., Shattuck, D., Salamon, G., Kudo, L. C., Ou, J. et al. (2004). Mutations in a human ROBO gene disrupt hindbrain axon pathway crossing and morphogenesis. Science 304, 1509-1513.
Jessell, T. M. (2000). Neuronal specification in the spinal cord: inductive signals and transcriptional codes. Nat Rev Genet 1, 90-29.
Ji, S. J., and Jaffrey, S. R. (2012). Intra-axonal translation of SMAD1/5/8 mediates retrograde regulation of trigeminal ganglia subtype specification. Neuron 74, 95-107.
Ji, S. J., and Jaffrey, S. R. (2013). Axonal transcription factors: novel regulators of growth cone-to-nucleus signaling. Dev Neurobiol 74, 245-258.
Jung, H., Gkogkas, C. G., Sonenberg, N., and Holt, C. E. (2014). Remote control of gene function by local translation. Cell 157, 26-40.
Jung, H., Yoon, B. C., and Holt, C. E. (2012). Axonal mRNA translation and local protein synthesis in nervous system assembly, maintenance, and repair. Nat Rev Neurosci 13, 308-324.
Kalil, K., and Dent, E. W. (2005). Touch and go: guidance cues signal to the growth cone cytoskeleton. Curr Opin Neurobiol 15, 521-526.
Keino-Masu, K., Masu, M., Hinck, L., Leonardo, E. D., Chan, S. S., Culotti, J. G., and Tessier-Lavigne, M. (1996). Deleted in Colorectal Cancer (DCC) encodes a netrin receptor. Cell 87, 175-185.
Kelleher, R. J., Govindarajan, A., and Tonegawa, S. (2004). Translational regulatory mechanisms in persistent forms of synaptic plasticity. Neuron 44, 59-73.
Kennedy, T. E., Serafini, T., de la Tone, J. R., and Tessier-Lavigne, M. (1994). Netrins are diffusible chemotropic factors for commissural axons in the embryonic spinal cord. Cell 78, 425-35.
Kessler, R. C., Chiu, W. T., Demler, O., Merikangas, K. R., and Walters, E. E. (2005). Prevalence, severity, and comorbidity of 12-month DSM-IV disorders in the National Comorbidity Survey Replication. Arch Gen Psychiatry 62, 617-627.
Kidd, T., Bland, K. S., and Goodman, C. S. (1999). Slit is the midline repellent for the robo receptor in Drosophila. Cell 96, 785-794.
Kidd, T., Brose, K., Mitchell, K. J., Fetter, F. D., Tessier-Lavigne, M., Goodman, C. S., and Tear, G. (1998). Roundabout controls axon crossing of the CNS midline and defines a novel subfamily of evolutionarily conserved guidance receptors. Cell 92, 205-215.
Kiick, K. L., Saxon, E., Tirrell, D. A., and Bertozzi, C. R. (2002). Incorporation of azides into recombinant proteins for chemoselective modification by the Staudinger ligation. Proc Natl Acad Sci USA 99, 19-24.
Kirsch, I., Deacon, B. J., Huedo-Medina, T. B., Scoboria, A., Moore, T. J., and Johnson, B. T. (2008). Initial severity and antidepressant benefits: a meta-analysis of data submitted to the Food and Drug Administration. PLoS Med 5, e45.
Kishi, M., Pan, V. A., Crump, J. G., and Sanes, J. R. (2005). Mammalian SAD kinases are required for neuronal polarization. Science 307, 929-932.
Kishi, N., and Macklis, J. D. (2010). MeCP2 functions largely cell-autonomously, but also non-cell-autonomously, in neuronal maturation and dendritic arborization of cortical pyramidal neurons. Exp Neurol 222, 51-58.
Klein, R. J., Zeiss, C., Chew, E. Y., Tsai. J. Y., Sackler, R. S., Haynes, C., Henning, A. K., SanGiovanni, J. P., Mane, S. M., Mayne, S. T., et al. (2005). Complement factor H polymorphism in age-related macular degeneration. Science 308, 385-389.
Koenig, E. (1967). Synthetic mechanisms in the axon. IV. In vitro incorporation of [3H]precursors into axonal protein and RNA. J Neurochem 14, 437-446.
Koenig, E., and Martin, R. (1996). Cortical plaque-like structures identify ribosomecontaining domains in the Mauthner cell axon. J Neurosci 16, 1400-1411.
Koenig, E., Martin, R., Titmus, M., and Sotelo-Silveira, J. R. (2000). Cryptic peripheral ribosomal domains distributed intermittently along mammalian myelinated axons. J Neurosci 20, 8390-8400.
Kolodkin, A. L., and Tessier-Lavigne, M. (2010). Mechanisms and molecules of neuronal wiring: a primer. Cold Spring Harb Perspect Biol 3, 1-14.
Kondrashov, N., Pusic, A., Stumpf, C. R., Shimizu, K., Hsieh, A. C., Xue, S., Ishijima, J., Shiroishi, T., and Barna, M. (2011). Ribosome-mediated specificity in Hox mRNA translation and vertebrate tissue patterning. Cell 145, 383-397.
Korn, E. D. (1982). Actin polymerization and its regulation by proteins from non-muscle cells. Physiol Rev 62, 672-737.
Kruger, R. P., Aurandt, J., and Guan, K. L. (2005). Semaphorins command cells to move. Nat Rev Mol Cell Biol 6, 789-800.
Krumm, N., O'Roak, B. J., Shendure, J., and Eichler, E. E. (2014). A de novo convergence of autism genetics and molecular neuroscience. Trends Neurosci 37, 95-105.
Krystal, J. H., and State, M. W. (2014). Psychiatric disorders: diagnosis to therapy. Cell 157, 201-214.
Kumamoto, T., Toma, K., Gunadi, McKenna, W. L., Kasukawa, T., Katzman, S., Chen, B., and Hanashima, C. (2013). Foxgl coordinates the switch from nonradially to radially migrating glutamatergic subtypes in the neocortex through spatiotemporal repression. Cell Rep 3, 931-945.
Kun, A., Otero, L., Sotelo-Silveira, J. R., and Sotelo, J. R. (2007). Ribosomal distributions in axons of mammalian myelinated fibers. J Neurosci Res 85, 2087-2098.
Lacroix, R., Robert, S., Poncelet, P., Kasthuri, R. S., Key, N. S., Dignat-George, F., and ISTH SSC Workshop. (2010). Standardization of platelet-derived microparticle enumeration by flow cytometry with calibrated beads: results of the International Society on Thrombosis and Haemostasis SSC Collaborative workshop. J Thromb Haemost 8, 2571-2574.
Lander, E. S., Linton, L. M., Birren, B., Nusbaum, C., Zody, M. C., Baldwin, J., Devon, K., Dewar, K, Doyle, M., Fitzhugh, W., et al. (2001). Initial sequencing and analysis of the human genome. Nature 409, 860-921.
Langmead, B., Trapnell, C., Pop, M., and Salzberg, S. L. (2009). Ultrafast and memoryefficient alignment of short DNA sequences to the human genome. Genome Biol, 10, R25.
Lebrand, C., Dent, E. W., Strasser, G. A., Lanier, L. M., Krause, M., Svitkina, T. M., Borisy, G. G., and Gertler, F. B. (2004). Critical role of Ena/VASP proteins for filopodia formation in neurons and in function downstream of netrin-1. Neuron 42, 37-49.
Lecompte, O., Ripp, R., Thierry, J. C., Moras, D., and Poch, O. (2002). Comparative analysis of ribosomal proteins in complete genomes: An example of reductive evolution at the domain scale. Nucleic Acids Res 30, 5382-5390.
Lein, E. S., Hawrylycz, M. J., Ao, N., Ayres, M., Bensinger, A., Bernard, A., Boe, A. F., Boguski, M. S.,
Brockway, K. S., Byrnes, E. J., et al. (2007). Genome-wide atlas of gene expression in the adult mouse brain. Nature 445, 168-176.
Letourneau, P. C. (1975a). Possible roles for cell-to-substratum adhesion in neuronal morphogenesis.
Dev Biol 44, 77-91.
Letourneau, P. C. (1975b). Cell-to-substratum adhesion and guidance of axonal elongation. Dev Biol 44, 92-101.
Letourneau, P. C. (1979). Cell-substratum adhesion of neurite growth cones, and its role in neurite elongation. Exp Cell Res 124, 127-138.
Letourneau, P. C. (1981). Immunocytochemical evidence for colocalization in neurite growth cones of actin and myo-sin and their relationship to cell-substratum adhesions. Dev Biol 85, 113-122.
Letourneau, P. C. (1983). Differences in the organization of actin in the growth cones compared with the neurites of cultured neurons from chick embryos. J Cell Biol 97, 963-973.
Leung, K. M., van Horck, F. P., Lin, A. C., Allison, R., Standart, N., and Holt, C. E. (2006), Asymmetrical β-actin mRNA translation in growth cones mediates attractive turning to netrin-1. Nat Neurosci 9, 1247-1256.
Leung, L. C., Urbancic, V., Baudet, M. L., Dwlvedy, A., Bayley, T. G., Lee, A. C., Harris, W. A., and Holt, C. E. (2013). Coupling of NF-protocadherin signaling to axon guidance by cue-induced translation. Nat Neurosci 16, 166-173.
Li, C., Bassell, G. J., and Sasaki, Y. Fragile X mental retardation protein is involved in protein synthesis-dependent collapse of growth cones induced by Semaphorin-3A. Front Neural Circuits 3, 11. doi:10.3389/neuron.04.011.2009.
Li, W., Lee, J., Vikis, H. G., Lee, S. H., Liu, G., Aurandt, J., Shen, T. L., Fearon, E. R., Guan, J. L., Han,
M. et al. (2004). Activation of FAK and Src are receptor-proximal events required for netrin signaling. Nature Neurosci 7, 1213-1221.
Lichtman, J. W., Pfister, H., and Shavit, N. (2014). The big data challenges of connectomics. Nat Neurosci 17, 1448-1454.
de Ligt, J., Willemsen, M. H., van Bon, B. W., Kleefstra, T., Yntema, H. G., Kroes, T., Vulto-van Silfhout, A. T., Koolen, D. A., de Vries, P., Gilissen, C., et al. (2012). Diagnostic exome sequencing in persons with severe intellectual disability. N Engl J Med 367, 1921-1929.
Lin, L., Lesnick, T. G., Maraganore, D. M., and Isacson, O. (2009). Axon guidance and synaptic maintenance: preclinical markers for neurodegenerative disease and therapeutics. Trends Neurosci 32, 142-149.
Liu, G., Beggs, H., Jurgensen, C., Park, H. T., Tang, H., Gorski, J., Jones, K. R., Reichardt, L. F., Wu, J., and Rao, Y. (2004). Netrin requires focal adhesion kinase and Src family kinases for axon outgrowth and attraction. Nature Neurosci 7, 1222-1232.
Liu, J., Krautzberger, A. M., Sui, S. H., Hofmann, O. M., Chen, Y., Baetscher, M., Grgic, I., Kumar, S., Humphreys, B. D., Hide, W. A., et al. (2014). Cell-specific translational profiling in acute kidney injury. J Clin Invest 124, 1242-1254.
Liu, J. P., Laufer, E., and Jessell, T. M. (2001). Assigning the positional identity of spinal motor neurons: rostrocaudal patterning of Hox-c expression by FGFs, Gdf11, and retinoids. Neuron 32, 997-1012.
Lobo, M. K., Karsten, S. L., Gray, M., Geschwind, D. H., and Yang, X. W. (2006). FACSarray profiling of striatal projection neuron subtypes in juvenile and adult mouse brains. Nat Neurosci 9, 443-452.
Lodato, S., Molyneaux, B. J., Zuccaro, E., Goff, L. A., Chen, H. H., Yuan, W., Meleski, A., Takahashi, E., Mahony, S., Rinn, J. L., et al. (2014). Gene co-regulation by Fezf2 selects neurotransmitter identity and connectivity of corticospinal neurons. Nat Neurosci 17, 1046-1054,
Lohse, K., Helmke, S. M., Wood, M. R., Quiroga, S., de la Houssaye, B. A., Miller, V. E., Negre-Aminou, P. and Pfenninger, K. H. (1996). Axonal origin and purity of growth cones isolated from fetal rat brain. Brain Res Dev Brain Res 96, 83-96.
Lowery, L. A., and Van Vactor, D. (2009). The trip of the tip: understanding the growth cone machinery. Nat Rev Mol Cell Biol, 10, 332-343.
Luo, L., and Flanagan, J. G. (2007). Development of continuous and discrete neural maps. Neuron 56, 284-300.
Luskin, M. B., Pearlman, A. L., and Sanes, J. R. (1998). Cell lineage in the cerebral cortex of the mouse studied in vivo and in vitro with a recombinant retrovirus. Neuron 1, 635-647.
Mack, E., Neubauer, A., and Brendel, C. (2007), Comparison of RNA yield from small cell populations sorted by flow cytometry applying different isolation procedures. Cytometry 71A, 404-409.
Madisen, L., Garner, A. R., Shimaoka, D., Chuong, A. S., Klapoetke, N. C., Li, L., van der Bourg, A., Niino, Y., Egolf, L., Monetti, C., et al. (2015). Transgenic mice for intersectional targeting of neural sensors and effectors with high specificity and performance. Neuron 85, 942-958.
Madisen, L., Zwingman, T. A., Sunkin, S. M., Oh, S. W., Zariwala, H. A., Gu, H., Ng, L. L., Palmiter, R. D., Hawrylycz, M. J., Jones, A. R., et al. (2010). A robust and high-throughput Cre reporting and characterization system for the whole mouse brain. Nat Neurosci 13, 133-140.
Maloney, M. T., and Bamburg, J. R. (2011), Mechanisms of neuronal growth cone guidance: An historical perspective. Devel Neurobio 71, 795-800.
Manji, H. K., Insel, T. R., and Narayan, V. A. (2014). Harnessing the informatics revolution for neuroscience drug R&D. Nat Rev Drug Discov 13, 561-562.
Manns, R. P., Cook, G. M., Holt, C. E., and Keynes, R. J. (2012). Differing semaphorin 3A concentrations trigger distinct signaling mechanisms in growth cone collapse. J Neurosci 32, 8554-8559.
Manolio, T. A., Collins, F. S., Cox, N. J., Goldstein, D. B., Hindorff, L. A., Hunter, D. J., McCarthy, M. I., Ramos, E. M., Cardon, L. R., Chakravarti, A., et al. (2009). Finding the missing heritability of complex diseases. Nature 461, 747-753.
Maraganore, D. M., de Andrade, M., Lesnick, T. G., Strain, K. J., Farrer, M. J., Rocca, W. A., Pant, P. V., Frazer, K. A., Cox, D. R., Ballinger, D. G. (2005). High-resolution wholegenome association study of Parkinson disease. Am J Hum Genet 77, 685-693.
Marquardt, T., Shirasaki, R., Ghosh, S., Andrews, S. E., Carter, N., Hunter, T., and Pfaff, S. L. (2005). Coexpressed EphA receptors and ephrin-A ligands mediate opposing actions on growth cone navigation from distinct membrane domains. Cell 121, 127-139.
Mathieson, W., and Thomas, G. A. (2013). Simultaneously extracting DNA, RNA, and protein using kits: Is sample quantity or quality prejudiced? Anal Biochem 433, 10-18.
Martin, R. E., and Bazan, N. G. (1992). Changing fatty acid content of growth cone lipids prior to synaptogenesis. J Neurochem 59, 318-325.
Martinez, S., Andreu, A., Mecklenburg, N., and Echevarria, D. (2013). Cellular and molecular basis of cerebellar development. Front Neuroanat 7, 1-12.
Mazumder, B., Sampath, P., Seshadri, V., Maitra, R. K., DiCorleto, P. E., and Fox, P. L. (2003). Regulated release of L13a from the 60S ribosome subunit as a mechanism of transcript-specific translational control. Cell 115, 187-198.
McCarroll, S. A., Feng, G., and Hyman, S. E. (2014). Genome-scale neurogenetics: methodology and meaning. Nat Neurosci 17, 756-763.
McLaughlin, T., Hindges, R., Yates, P. A., and O'Leary, D. D. M. (2003). Bifunctional action of ephrin-B1 as a repellent and attractant to control bidirectional branch extension in dorsal-ventral retinotopic mapping. Development 130, 2407-2418.
Meiri, K. F., and Gordon-Weeks, P. R. (1990). Gap-43 in growth cones is associated with areas of membrane that are tightly bound to substrate and is a component of a membrane skeleton subcellular fraction. J Neurosci 10, 256-266.
Memczak, S., Jens, M., Elefsinioti, A., Torti, F., Krueger, J., Rybak, A., Maier, L., Mackowiak, S. D., Gregersen, L. H., Munschauer, M., et al. (2013). Circular RNAs are a large class of animal RNAs with regulatory potential. Nature 495, 333-338.
Mercer, T. R., Dinger, M. E., Sunkin, S. M., Mehier, M. F., and Mattick, J. S. (2008). Specific expression of long noncoding RNAs in the mouse brain. Proc Natl Acad Sci USA 105, 716-721.
Merianda, T. T., Lin, A. C., Lam, J. S. Y., Vuppalanchi, D. Willis, D. E., Karin, N., Holt, C. E., and Twiss, J. L. (2009). A functional equivalent of endoplasmic reticulum and Golgi in axons for secretion of locally synthesized proteins. Moi Cell Neurosci 40, 128-142.
Michalski, A., Damoc, E., Lange, O., Denisov, E., Nolting, D., Muller, M., Viner, R., Schwartz, J., Reines P., Belford, M. et al. (2011). Ultra high resolution linear ion trap Orbitrap mass spectrometer (Orbitrap Elite) facilitates top down LC MS/MS and versatile peptide fragmentation modes. Mol Cell Proteomics 11, 0111.013698.
Milo, R. (2013). What is the total number of protein molecules per cell volume? A call to rethink some published values. Bioessays 35, 1050-1055.
Minis, A., Dahary, D., Manor, O., Leshkowitz, D., Pilpel, Y., and Yaron, A. (2014). Subcellular transcriptomics—Dissection of the mRNA composition in the axonal compartment of sensory neurons. Dev Neurobio 74, 365-381.
Minshew, N. J. and Williams, D. L. (2007) The new neurobiology of autism: cortex, connectivity, and neuronal organization. Arch Neurol 64, 945-950.
Mitchell, B. D., and Macklis, J. D. (2005). Large-scale maintenance of dual projections by callosal and frontal cortical projection neurons in adult mice. J Comp Neurol 482, 17-32.
Miyoshi, G., and Fishell, G. (2012). Dynamic FoxGl expression coordinates the integration of multipolar pyramidal neuron precursors into the cortical plate. Neuron 74, 1045-1058.
Molyneaux, B. J., Arlotta, P., Hirata, T., Hibi, M., and Macklis, J. D. (2005). Fezl is required for the birth and specification of corticospinal motor neurons. Neuron 47, 817-831.
Molyneaux, B. J., Arlotta, P., Menezes, Joao, R. L., and Macklis, J. D. (2007). Neuronal subtype specification in the cerebral cortex. Nat Rev Neurosci 8, 427-437.
Molyneaux, B. J., Arlotta, P., Fame, R. M., MacDonald, J. L., MacQuarrie, K. L., and Macklis, J. D. (2009). Novel subtype-specific genes identify distinct sub-populations of callosal projection neurons. J Neurosci 29, 12343-12354.
Molyneaux, B. J., Goff, L. A., Brettler, A. C., Chen, H. H., Brown, J. R., Hrvatin, S., Rinn, J. L, and Arlotta, P. (2015). DeCoN: Genome-wide analysis of in vivo transcriptional dynamics during pyramidal neuron fate selection in neocortex. Neuron 85, 275-288.
Morgan, J. L., and Lichtman, J. W. (2013). Why not connectomics? Nat Methods 10, 494-500.
Morse, S. M. J., Shaw, G., and Lamer, S. F. (2006). Concurrent mRNA and protein extraction from the same experimental sample using a commercially available column-based RNA preparation kit. BioTechniques 40, 54-58.
Nakai, J., and Kawasaki, Y. (1959). Studies on the mechanisms determining the course of the nerve fibers in tissue culture. I. The reaction of the growth cone to various obstructions. Z Zellforsch Mikrosc Anat 51, 108-122.
Nakamoto, M., Cheng, H. J., Friedman, G. C., McLaughlin, T., Hansen, M. J., Yoon, C. H., O'Leary, D. D., and Flanagan, J. G. (1996). Topographically specific effects of ELF-1 on retinal axon guidance in vitro and retinal axon mapping in vivo. Cell 86, 755-766.
Narayan, V. A., Mohwinckel, M., Pisano, G., Yang, M., and Manji, H. K. (2013). Beyond magic bullets: true innovation in health care. Nat Rev Drug Discov 12, 85-86.
Neale, B. M., Kou, Y., Liu, L., Ma'ayan, A., Samocha, K. E., Sabo, A., Lin, C. F., Stevens, C., Wang, L. S., Makarov, V., et al. (2012). Patterns and rates of exonic de novo mutations in autism spectrum disorders. Nature 485, 242-245.
Nédelee, S., Peljto, M., Shi, P., Amoroso, M. W., Kam, L. C., and Wichterle, H. (2012). Concentration-dependent requirement for local protein synthesis in motor neuron subtype-specific response to axon guidance cues. J Neurosci 32, 1496-1506.
Niquille, M., Garel, S., Mann, F., Hornung, J. P., Otsmane, B., Chevalley, S., Parras, C., Guillemot, F., Gaspar, P., Yanagawa, Y., et al. (2009) Transient neuronal populations are required to guide callosal axons: a role for semaphorin 3C. PLoS Biol 7, e1000230.
Nohe, A., Keating, E., Knaus, P., and Petersen, N. O. (2004). Signal transduction of bone morphogenetic protein receptors. Cell Signal 16, 291-299.
Nolte-'t Hoen, E. N., van der Vlist, E. J., Aalberts, M., Mertens, H. C., Bosch, B. J., Bartelink, W., Mastrobattista, E., van Gaal, E. V., et al. (2012). Quantitative and qualitative flow cytometric analysis of nanosized cell-derived membrane vesicles. Nanomedicine 8, 712-720.
Nozumi, M., Togano, T., Takahasi-Niki, K., Lu, J., Honda, A., Taoka, M., Shinkawa, T., Koga, H., Takeuchi, K., Isobe, T., et al. (2009). Identification of functional marker proteins in the mammalian growth cone. Proc Natl Acad Sci USA 106, 17211-17216.
Nugent, A. A., Kolpak, A. L., and Engle, E. C. (2012). Human disorders of axon guidance. Curr Opin Neurobiol 22, 837-843.
Oh, S. W., Harris, J. A., Ng, L., Winslow, B., Cain, N., Mihalas, S., Wang, Q., Lau, C., Kuan, L., Henry, A. M., et al. (2014). A mesoscale connectome of the mouse brain. Nature 508, 207-214.
Okada, A., Charron, F., Morin, S., Shin, D. S., Wong, K., Fabre, P. J., Tessier-Lavigne, M., and McConnell, S. K. (2006). Boc is a receptor for sonic hedgehog in the guidance of commissural axons. Nature 444, 369-373.
Orozco, A. F., and Lewis, D. E. (2010). Flow cytometric analysis of circulating microparticles in plasma. Cytometry A. 77, 502-514.
Pecho-Vrieseling, E., Sigrist, M., Yoshida, Y., Jesseli, T. M., and Arber, S. (2009). Specificity of sensory-motor connections encoded by Sema3e-Plxnd1 recognition. Nature 459, 842-846.
Pfenninger, K. H., Ellis, L., Johnson, M. P., Friedman, L. B., and Somlo, S. (1983). Nerve growth cones isolated from fetal brain: subcellular fractionation and characterization. Cell 35, 573-584.
Pfenninger, K. H. (2009). Plasma membrane expansion: a neuron's Herculean task. Nat Rev Neurosci 10, 251-261.
Poelmans, G., Franke, B., Pauls, D. L., Glennon, J. C., and Buitelaar, J. K. (2013). AKAPs integrate genetic findings for autism spectrum disorders. Transl Psychiatry 3, e270. Poliseno, L., Salmena, L., Zhang, J., Carver, B., Haveman, W. J., and Pandolfi, P. P. (2010). A coding-independent function of gene and pseudogene mRNAs regulate tumour biology. Nature 465, 1033-1018.
Pollard, T. D., and Borisy, G. G. (2003). Cellular motility driven by assembly and disassembly of actin filaments, Cell 112, 453-465.
Pollen, A. A., Nowakowski, T. J., Shuga, J., Wang, X., Leyrat, A. A, Lui, J. H., Li, N., Szpankowski, L., Fowler, B., Chen, P., et al. (2014). Low-coverage single-cell mRNA sequencing reveals cellular heterogeneity and activated signaling pathways in developing cerebral cortex. Nat Biotechnol 32, 1053-1058.
Polleux, F., Ince-Dun, G., and Ghosh, A. (2007). Transcriptional regulation of vertebrate axon guidance and synapse formation. Nat Rev Neurosci 8, 331-340.
Price, T. J., Flores, C. M., Cervero, F., and Hargreaves, K. M. (2006). The RNA binding and transport proteins staufen and fragile X mental retardation protein are expressed by rat primary afferent neurons and localize to peripheral and central axons. Neuroscience 141, 2107-2116.
Purcell, S. M., Moran, J. L., Fromer, M., Ruderfer, D., Solovieff, N., Roussos, P., O'Dushlaine, C., Chambert, K., Bergen, S. E., Kahler, A., et al. (2014). A polygenic burden of rare disruptive mutations in schizophrenia. Nature 506, 185-190.
Ramon y Cajal, S. (1995). Histology of the nervous system of man and vertebrates. New York, Oxford University Press.
Ren, X. R., Ming, G. L., Xie, Y., Hong, Y., Sun, D. M., Zhao, Z. Q., Feng, Z., Wang, Q., Shim, S., Chen, Z. F., et al. (2004). Focal adhesion kinase in netrin-1 signaling. Nature Neurosci 7, 1204-1212.
Reschke, M., Clohessy, J. G., Seitzer, N., Goldstein, D. P., Breitkopf, S. B., Schmolze, D. B., Ala, U., Asara, J. M., Beck, A. H., and Pandolfi, P. P. (2013). Characterization and analysis of the composition and dynamics of the mammalian riboproteome. Cell Rep 4, 1276-1287.
Rhee, J. M., Pirity, M. K., Lackan, C. S., Long, J. Z., Kondoh, G., Takeda, J., Hadjantonakis, A. K. (2006). In vivo imaging and differential localization of lipid-modified GFP-variant fusions in embryonic stem cells and mice. Genesis 44, 202-218.
Ripke, S., Neale, B. M., Corvin, A., Walters, J. T., Farh, K. H., Holman, P. A., Lee, P., Bulik-Sullivan, B., Collier, D. A., Huag, H., et al. (2014). Biological insights from 108 schizophrenia-associated genetic loci. Nature 511, 421-427.
Ripke, S., O'Dushlaine, C., Chambert, K., Moran, J. L., Uhler, A. K., Akterin, S., Bergen, S. E., Collins, A. L., Crowley, J. J., Fromer, M. et al. (2013). Genome-wide association analysis identifies 13 new risk loci for schizophrenia. Nat Genet 45, 1150-1159.
Robledo, S., Idol, R. A., Crimmins, D. L., Ladenson, J. H., Mason, P. J., and Bessler, M. (2008). The role of human ribosomal proteins in the maturation of rRNA and ribosome production. RNA 14, 1918-1929.
Roche, F. K., Marsick, B. M., and Letourneau, P. C. (2009). Protein synthesis in distal axons is not required for growth cone response to guidance cues. J Neurosci 29, 638-652.
Rolland, T., Tasan, M., Charloteaux, B., Pevzner, S. J., Zhong, Q., Sahni, N., Yi, S., Lemmens, I., Fontanillo, C., Mosca, R., et al. (2014). A proteome-scale map of the human interactome network. Cell 159, 1212-1226.
Romanes, G. J. (1964). The motor pools of the spinal cord. Prog Brain Res 11, 93-119.
Ross, A. F., Oleynikov, Y., Kislauskis, E. H., Taneja, K. L., and Singer, R. H. (1997). Characterization of a β-actin mRNA zipcode-binding protein. Mol Cell Biol 17, 2158-2165.
Rothberg, J. M., Jacobs, J. R., Goodman, C. S., and Artavanis-Tsakonas, S. (1990). Slit: an extracellular protein necessary for development of midline glia and commissural axon pathways contains both EGF and LRR domains. Genes Dev 4, 2169-2187.
Rouaux, C., and Arlotta, P. (2013). Direct lineage reprogramming of post-mitotic callosal neurons into corticofugal neurons in vivo. Nat Cell Biol 15, 214-221.
Saito, T., and Nakatsuji, N. (2001). Efficient gene transfer into the embryonic mouse brain using in vivo electroporation. Dev Biol 240, 237-246.
Salmena, L., Poliseno, L., Tay, Y., Kats, L., and Pandolfi, P. P. (2011). A ceRNA hypothesis: the Rosetta Stone of a hidden RNA language? Cell 146, 353-358.
Sander, M., Paydar, S., Ericson, J., Briscoe, J., Berber, E., German, M., Jessell, T. M., and Rubenstein, J. L. (2000). Ventral neural patterning by Nkx homebox genes: Nkx6.1 controls somatic motor neuron ventral interneuron fates. Genes Dev 14, 2134-2139.
Sanders, S. J., Murtha, M. T., Gupta, A. R., Murdoch, J. D., Raubeson, M. J., Whillsey, A. J., Ercan-Sencicek, A. G., DiLullo, N. M., Parikshak, N. N., Stein, J. L., et al. (2012). De novo mutations revealed by whole-exome sequencing are strongly associated with autism. Nature 485, 237-241.
Sanz, E., Yang, L., Su, T., Morris, D. R., McKnight, G. S., and Amieux, P. S. (2009). Celltype-specific isolation of ribosome-associated mRNA from complex tissues, Proc Natl Acad Sci USA 106, 13939-13944.
Sasaki, Y., Welshhans, K., Wen, Z., Yao, J., Xu, M., Goshima, Y., Zheng, J. Q., and Bassell, G. J. (2010). Phosphorylation of zipcode binding protein 1 is required for brainderived neurotrophic factor signaling of local b-actin synthesis and growth cone turning. J Neurosci 30, 9349-9358.
Sasaki, Y., Gross, C., Xing, L., Goshima, Y., and Bassell, G. J. (2013). Identification of axon-enriched microRNAs localized to growth cones of cortical neurons. Dev Neurobiol 74, 397-406.
Sauvageau, M., Goff, L. A., Lodato, S., Bonev, B., Groff, A. F., Gerhardinger, C., Sanchez-Gomez, D. B., Hacisuleyman, E., Li, E., Spence, M., et al. (2013). Multiple knockout mouse models reveal lincRNAs are required for life and brain development. eLife 2, e01749-e01749.
Sceniak, M. P., Lang, M., Enomoto, A. C., Howell, J. C., Hermes, D. J., and Katz, D. M. (2015). Mechanisms of functional hypoconnectivity in the medial prefrontal cortex of Mecp2 null mice. Cereb Cortex. Advance online publication. doi:10.1093/cercor/bhv002.
Schadt, E. E. (2009). Molecular networks as sensors and drivers of common human diseases. Nature 461, 218-223.
Schadt, E. E., and Bjorkegren, J. L. M. (2012). NEW: Network-enabled wisdom in biology, medicine, and health care. Sci Transl Med 4, 115v1.
Schuurmans, C., and Guillemot, F. (2002). Molecular mechanisms underlying cell fate specification in the developing telencephalon. Curr Opin Neurobiol 12, 26-34.
Schüz, A., and Palm, G. (1989). Density of neurons and synapses in the cerebral cortex of the mouse. J Comp Neurol 286, 442-455.
Serafini, T., Kennedy, T. E., Galko, M. J., Mirzayan, C., Jessell, T. M., and Tessier-Lavigne, M. (1994). The netrins define a family of axon outgrowth-promoting proteins homologous to C. elegans UNC-6. Cell 78, 409-424.
Shaw, G., and Bray, D. (1977). Movement and extension of isolated growth cones. Exp Cell Res 104, 55-62.
Shestakova, E. A., Singer, R. H., and Condeelis, J. (2001). The physiological significance of 3-actin mRNA localization in determining cell polarity and directional motility. Proc Natl Acad Sci USA 98, 7045-7050.
Shigeoka, T., Lu, B., and Holt, C. E. (2013). RNA-based mechanisms underlying axon guidance. J Cell Biot 202, 991-999.
Shirasaki, R., Katsumata, R., and Murakami, F. (1998). Change in chemoattractant responsiveness of developing axons at an intermediate target. Science 279, 105-107.
da Silva, S., and Wang, F. (2011). Retrograde neural circuit specification by targetderived neurotrophins and growth factors. Curr Opin Neurobiol 21, 61-67.
Sotelo, C. (2004). History of Neuroscience: The Neurotropic Theory of Santiago Ramón y Cajal. IBRO History of Neuroscience.
Sperry, R. W. (1951). Regulative factors in the orderly growth of neural circuits. Growth 15, 63-87.
Sperry, R. W. (1963). Chemoaffinity in the orderly growth of nerve fiber patterns and connections. Proc Natl Acad Sci 50, 703-710.
Srinivasan, K., Leone, D. P., Bateson, R. K., Dobreva, G., Kohwi, Y., Kohwi-Shigematsu, T., Grosschedl, R., and McConnell, S. K. (2012). A network of genetic repression and derepression specifies projection fates in the developing neocortex. Proc Natl Acad Sci USA 109, 19071-19078.
Stein, E., and Tessier-Lavigne, M. (2001). Hierarchical organization of guidance receptors: silencing of netrin attraction by slit through a Robo/DCC receptor complex. Science 291, 1928-1938.
Strack, R. L., Hein, B., Bhattacharyya, D., Hell, S. W., Keenan, R. J., and Glick, B. S. (2009). A rapidly maturing far-red derivative of DsRed-Express2 for whole-cell labeling. Biochemistry 48, 8279-8281.
Sugino, K., Hempel, C. M., Miller, M. N., Hattox, A. M., Shapiro, P., Wu, C., Huang, Z. J., Nelson, S. B. (2006). Molecular taxonomy of major neuronal classes in the adult mouse forebrain. Nat Neurosci 9, 99-107.
Sullivan, P. F., Daly, M. J., and O'Donovan, M. (2012). Genetic architectures of psychiatric disorders: the emerging picture and its implications. Nat Rev Genet 13, 537-551.
Sutton, M. A., and Schuman, E. M. (2006). Dendritic protein synthesis, synaptic plasticity, and memory. Cell 127, 49-58.
Svitkin, Y. V., Hahn, H., Gingras, A. C., Palmenberg, A. C., and Sonenberg, N. (1998). Rapamycin and wortmannin enhance replication of a defective encephalomyocarditis virus. J Virol 72, 5811-5819.
Tabata, H., and Nakajima, K. (2003). Multipolar migration: the third mode of radial neuronal migration in the developing cerebral cortex. J Neurosci 23, 9996-10001.
Tau, G. Z., and Peterson, B. S. (2010). Normal development of brain circuits. Neuropsychopharmacology 35, 147-168.
Taylor, A. M., Berchtold, N. C., Perreau, V. M., Tu, C. H., Jeon, N. L., and Cotman, C. W. (2009). Axonal mRNA in uninjured and regenerating cortical mammalian axons. J Neurosci 29, 4697-4707.
Tcherkezian, J., Brittis, P. A., Thomas, F., Roux, P. P., and Flanagan, J. G. (2010). Transmembrane receptor DCC associates with protein synthesis machinery and regulates translation. Cell 141, 632-644.
Tennyson, V. M. (1970). The fine structure of the axon and growth cone of the dorsal root neuroblast of the rabbit embryo. J Cell Biol 44, 62-79.
Terman, J. R., Mao, T., Pasterkamp, R. J., Yu, H. H., and Kolodkin, A. L. (2002). MICALs, a family of conserved flavoprotein oxidoreductases, function in plexin-mediated axonal repulsion. Cell 109, 887-900.
Tessier-Lavigne, M., and Goodman, C. S. (1996). The molecular biology of axon guidance. Science 274, 1123-1133.
Tischfield, M. A., Baris, H. N., Wu, C., Rudolph, G., Van Maldergem, L., He, W., Chan, W. M., Andrews, C., Demer, J. L., Robertson, R. L., et al. (2010). Human TUBB3 mutations perturb microtubule dynamics, kinesin interactions, and axon guidance. Cell 140, 74-87.
Tojima, T., Akiyama, H., Itofusa, R., Li, Y., Katayama, H., Miyawaki, A., and Kamiguchi, H. (2007). Attractive axon guidance involves asymmetric membrane transport and exocytosis in the growth cone. Nat Neurosci 10, 58-66.
Tojima, T., Hines, J. H., Henley, J. R., and Kamiguchi, H. (2011). Second messengers and membrane trafficking direct and organize growth cone steering. Nat Rev Neurosci 12, 191-203.
Tolosa, J. M., Schjenken, J. E., Civiti, T. D., Clifton, V. L., and Smith, R. (2007). Columnbased method to simultaneously extract DNA, RNA and proteins from the same sample. BioTechniques 43, 799-804.
Trapnell, C., Roberts, A., Goff, L., Pertea, G., Kim, D., Kelley, D. R., Pimentel, H., Salzberg, S. L., Rinn, J. L., and Pachter, L. (2012). Differential gene and transcript expression analysis of RNA-seq experiments with TopHat and Cufflinks. Nat Protoc 7, 562-578.
Trichas, G., Begbie, J., and Srinivas, S. (2008). Use of the viral 2A peptide for bicistronic expression in transgenic mice. BMC Biol 6, 40.
Tsiola, A., Hamzei-Sichani, F., Peterlin, Z., and Yuste, R. (2003). Quantitative morphologic classification of layer 5 neurons from mouse primary visual cortex. J Comp Neurol 461, 415-428.
Tsukita, S., and Ishikawa, H. (1976). Three-dimensional distribution of smooth endoplasmic reticulum in myelinated axons. J Electron Microsc 25, 141-149.
Tsukita, S., and Ishikawa, H. (1979). Morphological evidence for the involvement of the smooth endoplasmic reticulum in axonal transport. Brain Res 174, 315-318.
Vanderhorst, V. G., and Holstege, G. (1997). Organization of lumbosacral motoneuronal cell groups innervating hindlimb, pelvic floor, and axial muscles in the cat. J Comp Neurol 382, 46-76.
Van der Vlist, E. J., Nolte-'t Hoen, Stoorvogel, W., Arkesteijn, G. J., and Wauben, M. H. (2012). Fluorescent labeling of nano-sized vesicles released by cells and subsequent quantitative and qualitative analysis by high-resolution flow cytometry. Nat Protoc 7, 1311-1326.
Venter, J. C., Adams, M. D., Myers, E. W., Li, P. W., Mural, R. J., Sutton, G. G., Smith, H. O., Yandell, M., Evans, C. A., Holt, R. A., et al. (2001). The sequence of the human genome. Science 291, 1304-1351.
Verma, P., Chierzi, S., Codd, A. M., Campbell, D. S., Meyer, R. L., Holt, C. E., and Fawcett, J. W. (2005). Axonal protein synthesis and degradation are necessary for efficient growth cone regeneration. J Neurosci 25, 331-342.
Vitriol, E. A., and Zheng, J. Q. (2012). Growth cone travel in space and time: the cellular ensemble of cytoskeleton, adhesion, and membrane. Neuron 73, 1068-1081.
Walker, B. A., Hengst, U., Kim, H. J., Jeon, N. L., Schmidt, E. F., Heintz, N., Milner, T. A., and Jaffrey, S. R. (2012). Reprogramming axonal behavior by axon-specific viral transduction. Gene Therapy 19, 947-955.
Walsh, C., and Cepko, C. L. (1988). Clonally related cortical cells show several migration patterns. Science 241, 1342-1345.
Weiss, L. A., Arking, D. E., and The Gene Discovery Project of Johns Hopkins and the Autism Consortium (2009). A genome-wide linkage and association scan reveals novel loci for autism. Nature 461, 802-808.
White, J. G., Southgate, E., Thomson, J. N., and Brenner, S. (1986). The structure of the nervous system of the nematode Caenorhabditis elegans. Phil Trans R Soc Lond.B 314, 1-340.
Willis, D. E., Van Niekerk, E. A., Sasaki, Y., Mesngon, M., Merianda, T. T., Williams, G. G., Kendall, M., Smith, D. S., Bassell, G. J., and Twiss, J. L. (2007). Extracellular stimuli specifically regulate localized levels of individual neuronal mRNAs. J Cell Biol 178, 965-980.
Wu, K. Y., Hengst, U., Cox, L. J., Macosko, E. Z., Jeromin, A., Urquhart, E. R., and Jaffrey, S. R. (2005). Local translation of RhoA regulates growth cone collapse. Nature 436, 1020-1024.
Wu, K. Y., Zippin J. H., Huron, D. R., Kamenetsky, M., Hengst, U., Buck, J., Levin, L. R., and Jaffrey, S. R. (2006). Soluble adenylyl cyclase is required for netrin-1 signaling in nerve growth cones. Nature Neurosci 9, 1257-1264.
Xiao, Y., Camarillo, C., Ping, Y., Arana, T. B., Zhao, H., Thompson, P. M., Xu, C., Su, B. B., Fan, H., Ordonez, J., et al. (2014). The DNA methylome and transcriptome of different brain regions in schizophrenia and bipolar disorder. PLoS One 9, e95875.
Xue, S., and Barna, M. (2012). Specialized ribosomes: a new frontier in gene regulation and organismal biology. Nat Rev Mol Cell Biol 13, 355-369.
Yam, P. T., Langlois, S. D., Morin, S., and Charron, F. (2009). Sonic Hedgehog guides axons through a noncanonical, Src-family-kinase-dependent signaling pathway. Neuron 62, 349-362.
Yamada, K. M., Spooner, B. S., and Wessells, N. K. (1971). Ultrastructure and function of growth cones and axons of cultured nerve cells. J Cell Biol 49, 614-635.
Yao, J., Sasaki, Y., Wen, Z., Bassell, G. J., and Zheng, J. Q. (2006). An essential role for beta-actin mRNA localization and translation in Ca2+-dependent growth cone guidance. Nat Neurosci 9, 1265-1273.
Yazdani, U., and Terman, J. R. (2006). The semaphorins. Genome Biol 7, 211.
Ye, B., Zhang, Y., Song, W., Young, S. H., Jan, L. Y., and Jan, Y. N. (2007). Growing dendrites and axons differ in their reliance on the secretory pathway. Cell 130, 717-729.
Yi, J. J., Barnes, A. P., Hand, R., Polleux, F., and Ehlers, M. D. (2010). TGF-β signaling specifies axons during brain development. Cell 142, 144-157.
Yoon, B. C., Jung, H., Dwivedy, A., O'Hare, C. M., Zivraj, K. H., and Holt, C. E. (2012). Local translation of extranuclear lamin B promotes axon maintenance. Cell 148, 752-764.
You, X., Vlatkovic, I., Babic, A., Will, T., Epstein, I., Tushev, G., Akbalik, G., Wang, M., Glock, C., Qudenau, C., et al. (2015). Neural circular RNAs are derived from synaptic genes and regulated by development and plasticity. Nat Neurosci 18, 603-610.
Yu, T. W., and Bargmann, C. I. (2001). Dynamic regulation of axon guidance. Nat Neurosci 4, 1169-1176.
Yu, W., McDonnell, K., Taketo, M. M., and Bai, C. B. (2008). Wnt signaling determines ventral spinal cord fates in a time-dependent manner. Development 135, 3687-3696.
Zeisel, A., Manchado, A. B. M., Codeluppi, S., Lonnerberg, P., Manno, G. L., Jureus, A., Marques, S., Munguba, H., He, L., Betsholtz, C., et al. (2015). Cell types in the mouse cortex and hippocampus revealed by single-cell RNA-seq. Science 347, 1138-1142.
Zelená, J. (1970). Ribosome-like particles in myelinated axons of the rat. Brain Res 24, 359-363.
Zhang, C., and Darnell, R. B. (2011). Mapping in vivo protein-RNA interactions at singlenucleotide resolution from HITS-CLIP data. Nat Biotechnol 29, 607-614.
Zheng, J. Q., Kelly, T. K., Chang, B., Ryazantsev, S., Rajasekaran, A. K., Martin, K. C., and Twiss, J. L. (2001). A functional role for intra-axonal protein synthesis during axonal regeneration from adult sensory neurons. J Neurosci 21, 9291-9303.
Zheng, J. Q., and Poo, M. M. (2007). Calcium signaling in neuronal motility. Annu Rev Cell Dev Biol 23, 375-404.
Zheng, Y., Wildonger, J., Ye, B., Zhang, Y., Kita, A., Younger, S. H., Zimmerman, S., Jan, L. Y., and Jan, Y. N. (2008). Dynein is required for polarized dendritic transport and uniform microtubule orientiation in axons. Nat Cell Biol 10, 1172-1180.
Zhou, J., Wen, Y., Sui, Y. N., Liu, L., Richards, L. J., and Poo, M. M. (2013). Axon position within the corpus callosum determines contralateral cortical projection. Proc Natl Acad Sci USA 110, E2714-E2723.
Ziller, M. J., Edri, R., Yaffe, Y., Donaghey, J., Pop, R., Mallard, W., Issner, R., Gifford, C. A., Goren, A., Xing, J., et al. (2015). Dissecting neural differentiation regulatory networks through epigenetic footprinting. Nature 518, 355-359.
Zivraj, K. H., Tung, Y. C., Piper, M., Gumy, L., Fawcett, J. W., Yeo, G. S. H., and Holt, C. E. (2010). Subcellular profiling reveals distinct and developmentally regulated repertoire of growth cone mRNAs. J Neurosci 30, 15464-15478.

1-35. (canceled)

36. A method of treating a subject with a neurologic condition, the method comprising administering to the subject a composition comprising an effective amount of an agent that modulates expression of at least one nucleic acid enriched in neuron growth cones relative to neuron cell bodies, thereby treating the neurologic condition.

37. The method of claim 36, wherein the nucleic acid is selected from the group consisting of α-Tub, β-Tub, γ-Actin, Basp1, Crmp2, MAP1B, Ncam, Dynein, Basp1, L1cam, Contactin, Gprin1, Stxbp1, Dync1h1, Syn1, Cxadr, Gpm6a, Stx1b, Psmd1 and Mapre1.

38. The method of claim 36, wherein the agent is selected from the group consisting of small organic molecules, oligosaccharides, polysaccharides, peptides, proteins, peptide analogs, miRNA, siRNA, antisense RNA, and any combination thereof.

39. The method of claim 36, wherein the nucleic acid is more than about 2-fold enriched in the neuron growth cones relative to the neuron cell bodies.

40. The method of claim 39, wherein the neurologic condition is a neurodegenerative disorder.

41. The method of claim 40, wherein the neurodegenerative disorder is selected from the group consisting of Huntington's disease, dentatorubropallidoluysian atrophy, Kennedy's disease, spinocerebellar ataxia, fragile X syndrome, fragile XE mental retardation, Friedreich's ataxia, myotonic dystrophy, spinocerebellar ataxia type 8, spinocerebellar ataxia type 12, Alexander disease, Alper's disease, Alzheimer disease; amyotrophic lateral sclerosis, ataxia telangiectasia, Batten disease, Canavan disease, Cockayne syndrome, corticobasal degeneration, Creutzfeldt-Jakob disease, ischemia stroke, Krabbe disease, Lewy body dementia, multiple sclerosis, multiple system atrophy, Parkinson's disease, Pelizaeus-Merzbacher disease, Pick's disease, primary lateral sclerosis, Refsum's disease, Sandhoff disease, Schilder's disease, spinal cord injury; spinal muscular atrophy, Steele Richardson-Olszewski disease, and Tabes dorsalis.

42. The method of claim 36, wherein the neurologic condition is a neurodevelopmental disorder.

43. The method of claim 42, wherein the neurologic condition is selected from the group consisting of an autism spectrum disorder, schizophrenia, bipolar disorder, and Rett syndrome.

44. A method of forming or restoring a neuronal circuit, the method comprising contacting one or more of neuron growth cones with an effective amount of an agent that selectively targets the neuron growth cones relative to neuron cell bodies, causing the neuron growth cones to project towards their targets to form a synapse and thereby forming or restoring the neuronal circuit.

45. The method of claim 44, wherein the agent comprises one or more canonical cues.

46. The method of claim 45, wherein the canonical cues are selected from the group consisting of Netrins, Slits, Semaphorins and Ephrins.

47. The method of claim 44, wherein the agent binds to a marker selectively expressed by the neuron growth cone.

48. The method of claim 47, wherein the marker comprises Growth Associated Protein 43 (GAP-43).

49. A method of regenerating damaged neuronal circuitry in a subject, the method comprising contacting neuron growth cones of the subject with an effective amount of an agent that selectively targets at least one marker selectively expressed by the neuron growth cones relative to neuron cell bodies, causing the neuron growth cones to project towards their targets to form synapses and thereby restore the damaged neuronal circuity.

50. The method of claim 49, wherein the agent is selected from the group consisting of small organic molecules, oligosaccharides, polysaccharides, peptides, proteins, peptide analogs, miRNA, siRNA, antisense RNA, and any combination thereof.

51. The method of claim 49, wherein the agent comprises one or more canonical cues.

52. The method of claim 51, wherein the canonical cues are selected from the group consisting of Netrins, Slits, Semaphorins and Ephrins.

53. The method of claim 49, wherein the marker comprises a receptor.

54. The method of claim 49, wherein the agent binds to the marker selectively expressed by the neuron growth cone.

55. The method of claim 49, wherein the marker comprises GAP-43.
